(19) United States
(12) Patent Application Publication
(10) Pub. No.: US 2026/0124187 A1
(43) Pub. Date: May 7, 2026

(54) COMPOSITIONS FOR THE TREATMENT OF HYPERTENSION

(71) Applicant: The George Institute for Global Health, Randwick (AU)

(72) Inventors: Anthony RODGERS, Newtown (AU); Stephen MACMAHON, Newtown (AU)

(21) Appl. No.: 19/383,434

(22) Filed: Nov. 7, 2025

Related U.S. Application Data
(63) Continuation of application No. PCT/US2025/054060, filed on Nov. 4, 2025.
(60) Provisional application No. 63/716,555, filed on Nov. 5, 2024.

(51) Int. Cl. A61K 31/4422; A61K 31/404; A61K 31/4184; A61P 9/12

(52) U.S. Cl. CPC A61K 31/4422; A61K 31/404; A61K 31/4184; A61P 9/12

(57) ABSTRACT

Provided herein are pharmaceutical compositions that are useful for the treatment of hypertension comprising an angiotensin II receptor blocker, a diuretic, and a calcium channel blocker, wherein the dose of each component is below the lowest dose approved for the treatment of hypertension for the component.

### CROSS REFERENCE TO RELATED APPLICATIONS

This application is a continuation of International Application No. PCT/US2025/054060, filed Nov. 4, 2025, which claims the benefit and priority of U.S. Provisional Application No. 63/716,555, filed on Nov. 5, 2024, each of which is incorporated by reference herein in its entirety.

### BACKGROUND OF THE DISCLOSURE

High blood pressure, also known as hypertension, is a leading cause of preventable morbidity and mortality and it is well established that treatments that lower blood pressure (BP) are beneficial. However, despite the plethora of blood pressure lowering medicines available, many patients continue to have poor blood pressure control as evidenced by multiple large-scale population studies. Contributing factors for poor blood pressure control include poor adherence, complex guidelines recommending multiple up-titration steps, and treatment inertia.

Furthermore, the majority of treated patients receive only monotherapy, which has limited potency even at high doses where side effects are increased and tolerability reduced.

### SUMMARY OF THE DISCLOSURE

Recognized herein is a need for new treatments for lowering high blood pressure that are efficacious and tolerable. In one aspect, the present disclosure provides a method of treating a subject having hypertension, the method comprising administering a pharmaceutical composition comprising (a) telmisartan; (b) indapamide; and (c) amlodipine besylate, wherein said pharmaceutical composition is not administered in a subject who is prescribed aliskiren. In some embodiments, the pharmaceutical composition is formulated for oral administration. In some embodiments, the pharmaceutical composition is in the form of a pill, tablet, or capsule. In some embodiments, the treatment results in a systolic blood pressure (SBP) of less than about 140 mmHg. In some embodiments, the treatment results in a reduction of systolic blood pressure (SBP) of about 10 mmHg or greater. In some embodiments, the treatment results in a diastolic blood pressure (DBP) of less than about 90 mmHg. In some embodiments, the treatment results in a reduction of diastolic blood pressure (DBP) of about 5 mmHg or greater. In some embodiments, treatment results in a reduction in systolic blood pressure (SBP) that is greater than the reduction obtained with the full lowest hypertension therapeutic dose of any one of the (a), (b), and (c) in the pharmaceutical composition. In some embodiments, treatment results in a reduction in diastolic blood pressure (DBP) that is greater than the reduction obtained with the full lowest hypertension therapeutic dose of any one of (a), (b), and (c) in the pharmaceutical composition. In some embodiments, the treatment results in greater long-term tolerability and reduced risk of side effects when compared to treatment with the full lowest hypertension therapeutic dose of any one of (a), (b), and (c) in the pharmaceutical composition. In some embodiments, the treatment is an initial or first-line treatment of hypertension. In some embodiments, the treatment is not an initial or first-line treatment of hypertension. In some embodiments, the subject is administered with a first therapy comprising (i) telmisartan at a dose of about 10 mg, (ii) indapamide at a dose of about 0.625 mg, and (iii) amlodipine besylate at a dose of about EQ 1.25 mg base; (b) subsequent to the first therapy of (a), the subject is administered with a second therapy comprising (i) telmisartan at a dose of about 20 mg, (ii) indapamide at a dose of about 1.25 mg, and (iii) amlodipine besylate at a dose of about EQ 2.5 mg base; and (c) subsequent to the first therapy of (a) and the second therapy of (b), the subject is administered with a third therapy comprising (i) telmisartan at a dose of about 40 mg, (ii) indapamide at a dose of about 2.5 mg, and (iii) amlodipine besylate at a dose of about EQ 5 mg base. In some embodiments, a dose of indapamide is about 0.625 mg. In some embodiments, a dose of amlodipine besylate is about EQ 1.25 mg base. In some embodiments, a dose of telmisartan is about 10 mg. In some embodiments, a dose of indapamide is about 0.625 mg, a dose of amlodipine besylate is about EQ 1.25 mg base, and a dose of telmisartan is about 10 mg. In some embodiments, a dose of indapamide is about 1.25 mg. In some embodiments, a dose of amlodipine besylate is about EQ 2.5 mg base. In some embodiments, a dose of telmisartan is about 20 mg. In some embodiments, a dose of indapamide is about 1.25 mg, a dose of amlodipine besylate is about EQ 2.5 mg base, and a dose of telmisartan is about 20 mg. In some embodiments, a dose of indapamide is about 2.5 mg. In some embodiments, a dose of amlodipine besylate is about EQ 5 mg base. In some embodiments, a dose of telmisartan is about 40 mg. In some embodiments, a dose of indapamide is about 2.5 mg, a dose of amlodipine besylate is about EQ 5 mg base, and a dose of telmisartan is about 40 mg.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 shows the mean systolic blood pressure (mmHg) across time periods by treatment.

FIG. 2 shows the mean diastolic blood pressure (mmHg) across time periods by treatment.

FIG. 3 shows the mean heart rate across time periods by treatment.

FIG. 4 shows the process of screening and randomizing participants in the study of Example 7.

FIG. 5 shows the characteristics of randomized participants in the study of Example 7.

FIG. 6 shows the results of home and clinical mean blood pressure over time for the standard care and triple pill protocol groups of Example 7. Crude blood pressure and standard deviation at each visit are reported. Estimates for each participant at each visit are calculated from all available home blood pressure measurements since previous clinic visit. The number of participants with available blood pressure data at each visit is reported at the bottom of the figure. Numbers are only provided for times when clinic visits were performed, since clinic blood pressure was measured and home blood pressure measurement frequency was increased before these visits. These were purposefully differential; for example, the triple-pill protocol group had an extra visit in week 2 but no visits in months 4 and 5.

FIG. 7 shows primary and secondary effectiveness outcomes for the study of Example 7 over the course of Months 1, 2, 3 and 6. Mean difference and 95% Cl estimated from mixed models for repeated measures (MMRM). Model had blood pressure change from randomization as dependent variable, and covariates included treatment group, time (as categorical for months 1, 2, 3, and 6), treatment×time interaction, blood pressure at randomization (baseline), number of blood pressure-lowering drugs before randomization (naive vs monotherapy), age, sex, body mass index at baseline, and highest education and a participant random effect. For each participant home blood pressure change at clinic visit time point was calculated as the difference between mean home blood pressure at time point and home blood pressure at randomization. Mean home blood pressure at each time point was calculated using all available blood pressure measurements since previous clinic visit which both treatment group participants attended. All prespecified secondary effectiveness end points included except mean time to first week of home seated mean SBP less than 130 mm Hg and DBP less than 80 mm Hg; mean time at home seated mean SBP less than 130 mm Hg and DBP less than 80 mm Hg from randomization to month 6. Risk difference and 95% Cl are from binomial regression models with blood pressure control as dependent variable and covariates treatment group, baseline blood pressure and patient characteristics at baseline, age, sex, body mass index, number of blood pressure medications before randomization, and education.

FIG. 8 shows primary outcome of data associated with Example 7 divided by subgroup.

FIG. 9 depicts a sensitivity analysis for the primary outcome of reduction in Home Mean systolic blood pressure (SBP) for the study of Example 7 from randomization to Month 6.

FIG. 10 depicts home and clinical blood pressure and difference between the triple pill and standard care protocols for the study of Example 7 for Months 1, 2, 3 and 6.

FIG. 11 depicts home and clinical blood pressure and difference between the groups for the study of Example 7 for Months 1, 2, 3 and 6.

FIG. 12 depicts safety outcomes for the triple-pill and standard care protocol groups for the study of Example 7 for from randomization to month 6 and at month 6.

FIG. 13 depicts laboratory parameters for the triple pill and standard care groups of the study of Example 7.

FIG. 14 depicts participant participation in each group of blood pressure-lowering drug regimens at each follow-up visit in the study of Example 7.

FIG. 15 depicts the process or screening and assignment for participants in the study of Example 8.

FIG. 16 depicts baseline characteristics of participants across different groups in the study of Example 8.

FIG. 17 depicts the systolic and diastolic blood pressures of participants in the different groups of the study of Example 8.

FIG. 18 depicts the home and clinic systolic and diastolic blood pressures of participants in the different groups of the study of Example 8.

FIG. 19 depicts the home and clinic systolic and diastolic blood pressures of participants in the different groups of the study of Example 8.

FIG. 20 summarizes reasons and rates of treatment withdrawal in the different groups of the study of Example 8.

FIG. 21 shows the primary efficacy outcome (difference in change in home systolic blood pressure) within different demographic groups of the study of Example 8.

FIG. 22 shows the secondary efficacy outcome (difference in change in clinic SBP at week 4) within different demographic groups of the study of Example 8.

FIG. 23 shows mean potassium level for the triple- and dual-therapy groups of Example 8 at screening, baseline, week 6 and week 12.

### DETAILED DESCRIPTION OF THE DISCLOSURE

Provided herein are pharmaceutical compositions for the treatment of hypertension comprising an angiotensin II receptor blocker, a diuretic, and a calcium channel blocker, wherein the dose of each component may be below the lowest dose approved dose for the treatment of hypertension. Pharmaceutical compositions as described herein may be prescribed with certain contraindications. For instance, pharmaceutical compositions as described herein may not be administered to a subject who is prescribed aliskiren. For instance, pharmaceutical compositions as described herein may not be administered to a subject who is taking aliskiren. The present disclosure recognizes the technical effects of low-dose combination therapy set forth herein, including but not limited to, the use of low doses to avoid or ameliorate side effects while retaining or improving benefits, the synergistic therapeutic benefits of certain drug combinations, and the early introduction of combination therapy to improve therapeutic effects. Described herein are low-dose combination compositions for the treatment of hypertension, including the initial or first-line treatment of hypertension.

### Certain Terminology

As used herein and in the appended claims, the singular forms “a,” “and,” and “the” include plural referents unless the context clearly dictates otherwise. Thus, for example, reference to “an agent” includes a plurality of such agents, and reference to “the composition” includes reference to one or more compositions (or to a plurality of compositions) and equivalents thereof known to those skilled in the art, and so forth. When ranges are used herein for physical properties, such as molecular weight, or chemical properties, such as chemical formulae, all combinations and subcombinations of ranges and specific embodiments therein are intended to be included. The term “about” when referring to a number or a numerical range means that the number or numerical range referred to is an approximation within experimental variability (or within statistical experimental error), and thus the number or numerical range may vary between 1% and 10% of the stated number or numerical range. The term “comprising” (and related terms such as “comprise” or “comprises” or “having” or “including”) is not intended to exclude that in other certain embodiments, for example, an embodiment of any composition of matter, composition, method, or process, or the like, described herein, may “consist of” or “consist essentially of” the described features.

### Definitions

As used in the specification and appended claims, unless specified to the contrary, the following terms have the meaning indicated below.

“Pharmaceutically acceptable salt” as used herein includes both acid and base addition salts. In some embodiments, the pharmaceutically acceptable salt of any one of the compounds described herein is the form approved for use by the US Food and Drug Administration. Preferred pharmaceutically acceptable salts of the compounds described herein are pharmaceutically acceptable acid addition salts and pharmaceutically acceptable base addition salts.

“Pharmaceutically acceptable acid addition salt” refers to those salts which retain the biological effectiveness and properties of the free bases, which are not biologically or otherwise undesirable, and which are formed with inorganic acids such as hydrochloric acid, hydrobromic acid, sulfuric acid, nitric acid, phosphoric acid, hydroiodic acid, hydrofluoric acid, phosphorous acid, and the like. Also included are salts that are formed with organic acids such as aliphatic mono- and dicarboxylic acids, phenyl-substituted alkanoic acids, hydroxy alkanoic acids, alkanedioic acids, aromatic acids, aliphatic and aromatic sulfonic acids, etc. and include, for example, acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Exemplary salts thus include sulfates, pyrosulfates, bisulfates, sulfites, bisulfites, nitrates, phosphates, monohydrogenphosphates, dihydrogenphosphates, metaphosphates, pyrophosphates, chlorides, bromides, iodides, acetates, trifluoroacetates, propionates, caprylates, isobutyrates, oxalates, malonates, succinate suberates, sebacates, fumarates, maleates, mandelates, benzoates, chlorobenzoates, methylbenzoates, dinitrobenzoates, phthalates, benzenesulfonates, toluenesulfonates, phenylacetates, citrates, lactates, malates, tartrates, methanesulfonates, and the like. Also contemplated are salts of amino acids, such as arginates, gluconates, and galacturonates (see, for example, Berge S. M. et al., “Pharmaceutical Salts,” Journal of Pharmaceutical Science, 66:1-19 (1997), which is hereby incorporated by reference in its entirety). Acid addition salts of basic compounds may be prepared by contacting the free base forms with a sufficient amount of the desired acid to produce the salt according to methods and techniques with which a skilled artisan is familiar.

“Pharmaceutically acceptable base addition salt” refers to those salts that retain the biological effectiveness and properties of the free acids, which are not biologically or otherwise undesirable. These salts are prepared from addition of an inorganic base or an organic base to the free acid. Pharmaceutically acceptable base addition salts may be formed with metals or amines, such as alkali and alkaline earth metals or organic amines. Salts derived from inorganic bases include, but are not limited to, sodium, potassium, lithium, ammonium, calcium, magnesium, iron, zinc, copper, manganese, aluminum salts and the like. Salts derived from organic bases include, but are not limited to, salts of primary, secondary, and tertiary amines, substituted amines including naturally occurring substituted amines, cyclic amines and basic ion exchange resins, for example, isopropylamine, trimethylamine, diethylamine, triethylamine, tripropylamine, ethanolamine, diethanolamine, 2-dimethylaminoethanol, 2-diethylaminoethanol, dicyclohexylamine, lysine, arginine, histidine, caffeine, procaine, N,N-dibenzylethylenediamine, chloroprocaine, hydrabamine, choline, betaine, ethylenediamine, ethylenedianiline, N-methylglucamine, glucosamine, methylglucamine, theobromine, purines, piperazine, piperidine, N-ethylpiperidine, polyamine resins and the like. See Berge et al., supra.

As used herein, “hydrates” are compounds that contain either stoichiometric or non-stoichiometric amounts of water, and, in some embodiments, are formed during the process of crystallization with water. Hydrates are meant to include the hydrates of any one of the compounds described herein that is approved for use by the US Food and Drug Administration.

The term “acceptable” with respect to a formulation, composition or ingredient, as used herein, means having no persistent detrimental effect on the general health of the subject being treated.

The terms “administer,” “administering,” “administration,” and the like, as used herein, refer to the methods that may be used to enable delivery of compounds or compositions to the desired site of biological action. These methods include, but are not limited to oral routes, intraduodenal routes, parenteral injection (including intravenous, subcutaneous, intraperitoneal, intramuscular, intravascular or infusion), topical and rectal administration. Those of skill in the art are familiar with administration techniques that can be employed with the compounds and methods described herein. In some embodiments, the compounds and compositions described herein are administered orally.

The term “subject” or “patient” encompasses mammals. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. In one aspect, the mammal is a human.

As used herein, “treatment” or “treating” or “palliating” or “ameliorating” are used interchangeably herein. These terms refers to an approach for obtaining beneficial or desired results including but not limited to therapeutic benefit and/or a prophylactic benefit. “Therapeutic benefit” is meant to include the eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient may still be afflicted with the underlying disorder. For prophylactic benefit, the compositions may be administered to a patient at risk of developing a particular disease, or to a patient reporting one or more of the physiological symptoms of a disease, even though a diagnosis of this disease may not have been made.

### Triple Compositions

Described herein are pharmaceutical compositions comprising one or more therapeutic agents for treatment of hypertension. In some embodiments, the pharmaceutical composition comprises a combination of one or more therapeutic agents at low doses. In some embodiments, the pharmaceutical composition comprises an angiotensin II receptor blocker. In some embodiments, the pharmaceutical composition comprises a diuretic. In some embodiments, the pharmaceutical composition comprises a calcium channel blocker. In some embodiments, the pharmaceutical composition comprises (a) an angiotensin II receptor blocker; (b) a diuretic; and (c) a calcium channel blocker. In some embodiments, the dose of each (a), (b), and (c) is from about 40% to about 200% of the lowest hypertension therapeutic dose (LHTD) for each of the (a), (b), and (c). In some embodiments, the dose of each (a), (b), and (c) is from about 40% to about 80% of the lowest hypertension therapeutic dose (LHTD) for each of the (a), (b), and (c). In some embodiments, the dose of each (a), (b), and (c) is from about 40% to about 60% of the lowest hypertension therapeutic dose (LHTD) for each of the (a), (b), and (c). In some embodiments, the dose of each (a), (b), and (c) is from about 50% to about 100% of the lowest hypertension therapeutic dose (LHTD) for each of the (a), (b), and (c). In some embodiments, the dose of each (a), (b), and (c) is about 50% of the lowest hypertension therapeutic dose (LHTD) for each of the (a), (b), and (c). In some embodiments, the dose of each (a), (b), and (c) is about 100% of the lowest hypertension therapeutic dose (LHTD) for each of the (a), (b), and (c). In some embodiments, the dose of each (a), (b), and (c) is about 200% of the lowest hypertension therapeutic dose (LHTD) for each of the (a), (b), and (c). In some embodiments, the dose of each (a), (b), and (c) is about 60% to about 80% of the lowest hypertension therapeutic dose (LHTD) for each of the (a), (b), and (c). In some embodiments, the dose of each (a), (b), and (c) is about 66% of the lowest hypertension therapeutic dose (LHTD) for each of the (a), (b), and (c). In some embodiments, the lowest hypertension therapeutic dose (LHTD) and the corresponding proposed dose and proposed dose range for the following compounds are as described in the following table:

TABLE 1

| | Lowest | |
|---|---|---|
| | Hypertension | Proposed Dose |
| | Therapeutic | Range (mg) |
| Agent | Dose (mg) | (% LHTD) |
| Amlodipine | EQ 2.5 mg base | EQ 1 mg base - EQ |
| besylate | | 6.25 mg base (40%- |
| | | 250% of LHTD) |
| Chlorthalidone | 25 | 10-62.5 (40%- |
| | | 250% of LHTD) |
| Hydrochlorothiazide | 12.5 | 5-31.25 (40%- |
| | | 250% of LHTD) |
| Indapamide | 1.25 | 0.5-3.125 (40%- |
| | | 250% of LHTD) |
| Irbesartan | 75 | 30-187.5 (40%- |
| | | 250% of LHTD) |
| Telmisartan | 20 | 8-50 (40%-250% of |
| | | LHTD) |

In some embodiments, the pharmaceutical composition comprises one or more blood pressure lowering active agents, wherein the blood pressure lowering active agents comprise an angiotensin II receptor blocker, a diuretic, a calcium channel blocker, or any combination thereof.

In some embodiments, the pharmaceutical compositions disclosed herein are free of an angiotensin converting enzyme inhibitor (ACE inhibitor) or the pharmaceutically acceptable salt thereof. In some embodiments, the angiotensin converting enzyme inhibitor includes but is not limited to benazepril, captopril, enalapril, fosinopril, lisinopril, moexipril, perindopril, quinapril, ramipril, trandolapril or the pharmaceutically acceptable salt or hydrate thereof.

Also described herein are pharmaceutical compositions consisting of (a) an angiotensin II receptor blocker; (b) a diuretic; and (c) a calcium channel blocker; wherein the dose of each (a), (b), and (c) is from about 40% to about 80% of the lowest hypertension therapeutic dose (LHTD) for each of the (a), (b), and (c). In some embodiments, the dose of each (a), (b), and (c) is from about 40% to about 60% of the lowest hypertension therapeutic dose (LHTD) for each of the (a), (b), and (c). In some embodiments, the dose of each (a), (b), and (c) is from about 50% to about 200% of the lowest hypertension therapeutic dose (LHTD) for each of the (a), (b), and (c). In some embodiments, the dose of each (a), (b), and (c) is from about 50% to about 100% of the lowest hypertension therapeutic dose (LHTD) for each of the (a), (b), and (c). In some embodiments, the dose of each (a), (b), and (c) is about 50% of the lowest hypertension therapeutic dose (LHTD) for each of the (a), (b), and (c). In some embodiments, the dose of each (a), (b), and (c) is about 100% of the lowest hypertension therapeutic dose (LHTD) for each of the (a), (b), and (c). In some embodiments, the dose of each (a), (b), and (c) is about 200% of the lowest hypertension therapeutic dose (LHTD) for each of the (a), (b), and (c). In some embodiments, the dose of each (a), (b), and (c) is about 60% to about 80% of the lowest hypertension therapeutic dose (LHTD) for each of the (a), (b), and (c). In some embodiments, the dose of each (a), (b), and (c) is about 66% of the lowest hypertension therapeutic dose (LHTD) for each of the (a), (b), and (c).

In some embodiments, the pharmaceutical compositions disclosed herein achieve a significant blood pressure reduction in a subject with modestly elevated blood pressure. In some embodiments, the pharmaceutical compositions disclosed herein achieve a significant blood pressure reduction in a subject with modestly elevated blood pressure with minimum, insignificant, or no side effects.

### Beta-Blockers

In some embodiments, the pharmaceutical compositions disclosed herein comprise a beta-blocker or pharmaceutically acceptable salt thereof. In some embodiments, the pharmaceutical compositions disclosed herein are essentially free of a beta-blocker or the pharmaceutically acceptable salt thereof. In some embodiments, beta-blockers are compounds that inhibit the receptor sites for the endogenous catecholamines epinephrine (adrenaline) and norepinephrine (noradrenaline), on adrenergic beta receptors of the sympathetic nervous system. In some embodiments, beta-blockers include but are not limited to beta-adrenergic blocking agents, beta antagonists, beta-adrenergic antagonists, beta-adrenoreceptor antagonists, or beta-adrenergic receptor antagonists. In some embodiments, beta-blockers inhibit activation of all types of β-adrenergic receptors. In some embodiments, beta-blockers inhibit both β-adrenergic receptors and α-adrenergic receptors. In some embodiments, beta-blockers are selective for one of the following beta receptors: β1, β2 and β3 receptors.

In some embodiments, the beta-blocker is a non-selective beta-adrenoceptor antagonist. Examples of non-selective beta-adrenoceptor antagonists include, but are not limited to, pindolol, propranolol, oxprenolol, sotalol, timolol, carteolol, penbutolol, and nadolol. In some embodiments, the beta-blocker is a compound with combined β- and α-adrenoceptor blocking action. Suitable examples include, but are not limited to, carvedilol, bucindolol, and labetolol. In some embodiments, the beta-blocker is a Bi-selective adrenoceptor antagonist. Examples of β₁ selective adrenoceptor antagonist include, but are not limited to, atenolol, bisoprolol, betaxolol, metoprolol, celiprolol, esmolol, nebivolol, and acebutolol. In some embodiments, the beta-blocker is a β₂-selective adrenoceptor antagonist, such as butaxamine.

In some embodiments, the beta-blocker is acebutolol, atenolol, betaxolol, bisoprolol, carteolol, esmolol, penbutolol, metoprolol, nadolol, nebivolol, pindolol, sotalol, propranolol, carvedilol, labetalol, timolol, esmolol, celiprolol, oxprenolol, levobunolol, practolol, metipranolol, landiolol, bopindolol, pronethalol, butaxamine, bevantolol, tertatolol, arotinolol, levobetaxolol, befunolol, amosulalol, tilisolol, or the pharmaceutically acceptable salt or hydrate thereof. In some embodiments, the beta-blocker is acebutolol, atenolol, betaxolol, bisoprolol, carteolol, esmolol, penbutolol, metoprolol, nadolol, nebivolol, pindolol, sotalol, propranolol, carvedilol, labetalol or the pharmaceutically acceptable salt or hydrate thereof. In some embodiments, the beta-blocker is acebutolol, atenolol, betaxolol, bisoprolol, celiprolol, oxprenolol, metoprolol, nadolol, nebivolol, pindolol, propranolol, carvedilol, labetalol, timolol, or the pharmaceutically acceptable salt or hydrate thereof. In some embodiments, the beta-blocker is atenolol. In some embodiments, the beta-blocker is bisoprolol or the pharmaceutically acceptable salt.

### Lipid-Regulating Agent

In some embodiments, the pharmaceutical compositions disclosed comprise a lipid-regulating agent, a platelet function altering agent, a serum homocysteine lowering agent, or a combination thereof. In some embodiments, the pharmaceutical compositions disclosed herein are essentially free of a lipid-regulating agent, a platelet function altering agent, a serum homocysteine lowering agent, or a combination thereof.

In some embodiments, the pharmaceutical compositions disclosed herein are essentially free of a lipid-regulating agent. In some embodiments, the lipid-regulating agent is a 3-hydroxy-3-methylglutaryl coenzyme A (HMG CoA) reductase inhibitor, also called a statin. In some embodiments, the lipid-regulating agent is atorvastatin, simvastatin, cerivastatin, fluvastatin, or pravastatin. In some embodiments, the lipid-regulating agent is atorvastatin or simvastatin. In some embodiments, the lipid-regulating agent is atorvastatin. In some embodiments, the lipid-regulating agent is simvastatin.

### Platelet Function Altering Agent

In some embodiments, the pharmaceutical compositions disclosed herein comprise a platelet function altering agent. In some embodiments, the pharmaceutical compositions disclosed herein are essentially free of a platelet function altering agent. In some embodiments, the platelet function altering agent is aspirin, ticlopidine, dipyridamole, or clopidogrel. In some embodiments, the platelet function altering agent is a glycoprotein IIb/IIIa receptor inhibitor, such as abciximab. In some embodiments, the platelet function altering agent is a non-steroidal anti-inflammatory drug, such as ibuprofen. In some embodiments, the platelet function altering agent is aspirin, ticlopidine, dipyridamole, clopidogrel, abciximab, or ibuprofen. In some embodiments, the platelet function altering agent is aspirin.

### Serum Homocysteine Lowering Agent

In some embodiments, the pharmaceutical compositions disclosed herein comprise a serum homocysteine lowering agent. In some embodiments, the pharmaceutical compositions disclosed herein are essentially free of a serum homocysteine lowering agent. In some embodiments, the serum homocysteine lowering agent is folic acid, vitamin B6, vitamin B12, or a combination thereof. In some embodiments, the serum homocysteine lowering agent is folic acid.

### Angiotensin II Receptor Antagonist Blocker

The present disclosure provides compositions comprising an angiotensin II receptor antagonist or blocker (ARB). ARBs are compounds that may modulate the action of angiotensin II by preventing angiotensin II from binding to angiotensin II receptors on the muscles surrounding blood vessels. In some embodiments, angiotensin II receptor blocker is losartan, valsartan, candesartan, eprosartan, irbesartan, telmisartan, or the pharmaceutically acceptable salt or hydrate thereof. In some embodiments, the angiotensin II receptor blocker is losartan. In some embodiments, the angiotensin II receptor blocker is valsartan. In some embodiments, the angiotensin II receptor blocker is candesartan. In some embodiments, the angiotensin II receptor blocker is eprosartan. In some embodiments, the angiotensin II receptor blocker is irbesartan. In some embodiments, the angiotensin II receptor blocker is telmisartan.

### Diuretics

The present disclosure provides compositions comprising a diuretic. Diuretics refer to compounds that increase urinary flow rate. Diuretics may be classified by chemical structure (thiazide diuretics and thiazide-like diuretics), site of action (such as loop diuretics) or pharmacologic effect (such as osmotic diuretics, carbonic anhydrase inhibitors, and potassium sparing diuretics).

In some embodiments, the pharmaceutical compositions disclosed herein comprise a thiazide diuretic. In some embodiments, the pharmaceutical compositions disclosed herein comprise a thiazide-like diuretic. In some embodiments, the pharmaceutical compositions disclosed herein comprise a loop diuretic. In some embodiments, the pharmaceutical compositions disclosed herein comprise an osmotic diuretic. In some embodiments, the pharmaceutical compositions disclosed herein comprise a carbonic anhydrase inhibitor. In some embodiments, the pharmaceutical compositions disclosed herein comprise a potassium sparing diuretic.

### Thiazide Diuretics

As used herein, thiazide diuretics refer to compounds that may contain the benzothiadiazine molecular structure. In some embodiments, thiazide diuretics inhibit sodium and chloride reabsorption in the distal tubule of the kidney, which results in increased urinary excretion of sodium and water. Examples of thiazide diuretics include, but are not limited to, altizide, bendroflumethiazide, chlorothiazide, cyclopenthiazide, cyclothiazide, epitizide, hydrochlorothiazide, hydroflumethiazide, mebutizide, methyclothiazide, polythiazide, and trichlormethiazide.

In some embodiments, the thiazide diuretic is altizide, bendroflumethiazide, chlorothiazide, cyclopenthiazide, cyclothiazide, epitizide, hydrochlorothiazide, hydroflumethiazide, mebutizide, methyclothiazide, polythiazide, trichlormethiazide, or the pharmaceutically acceptable salt or hydrate thereof. In some embodiments, the thiazide diuretic is altizide. In some embodiments, the thiazide diuretic is bendroflumethiazide. In some embodiments, the thiazide diuretic is chlorothiazide. In some embodiments, the thiazide diuretic is cyclopenthiazide. In some embodiments, the thiazide diuretic is cyclothiazide. In some embodiments, the thiazide diuretic is epitizide. In some embodiments, the thiazide diuretic is hydrochlorothiazide. In some embodiments, the thiazide diuretic is hydroflumethiazide. In some embodiments, the thiazide diuretic is mebutizide. In some embodiments, the thiazide diuretic is methyclothiazide. In some embodiments, the thiazide diuretic is polythiazide. In some embodiments, the thiazide diuretic is trichlormethiazide.

### Thiazide-Like Diuretics

As used herein, a thiazide-like diuretic is a sulfonamide diuretic that has similar physiological properties to a thiazide diuretic, but does not have the chemical properties of a thiazide (i.e. does not have the benzothiadiazine core). Examples of thiazide-like diuretics include, but are not limited to, quinethazone, clopamide, chlorthalidone, mefruside, clofenamide, metolazone, meticrane, xipamide, indapamide, clorexolone, and fenquizone.

In some embodiments, the thiazide-like diuretic is quinethazone, clopamide, chlorthalidone, mefruside, clofenamide, metolazone, meticrane, xipamide, indapamide, clorexolone, fenquizone, or the pharmaceutically acceptable salt or hydrate thereof. In some embodiments, the thiazide-like diuretic is quinethazone. In some embodiments, the thiazide-like diuretic is clopamide. In some embodiments, the thiazide-like diuretic is chlorthalidone. In some embodiments, the thiazide-like diuretic is mefruside. In some embodiments, the thiazide-like diuretic is clofenamide. In some embodiments, the thiazide-like diuretic is metolazone. In some embodiments, the thiazide-like diuretic is meticrane. In some embodiments, the thiazide-like diuretic is xipamide. In some embodiments, the thiazide-like diuretic is indapamide or the hydrate thereof. In some embodiments, the thiazide-like diuretic is indapamide. In some embodiments, the thiazide-like diuretic is clorexolone. In some embodiments, the thiazide-like diuretic is fenquizone.

### Loop Diuretics

As used herein, loop diuretics are compounds that act on the Na+/K+/2Cl— cotransporter in the thick ascending loop of Henle to inhibit sodium, chloride and potassium reabsorption. Examples of loop diuretics include, but are not limited to, furosemide, bumetanide, etacrynic acid, etozolin, muzolimine, ozolinone, piretanide, tienilic acid, and torasemide. In some embodiments, the loop diuretic is furosemide, bumetanide, etacrynic acid, etozolin, muzolimine, ozolinone, piretanide, tienilic acid, torasemide, or a pharmaceutically acceptable salt or hydrate thereof.

### Other Diuretics

Osmotic diuretics are compounds that cause water to be retained within the proximal tubule and descending limb of loop of Henle. In some embodiments, the osmotic diuretic expands fluid and plasma volume and increases blood flow to the kidney. Examples include but are not limited to mannitol and glycerol.

### Potassium Sparing Diuretics

Potassium sparing diuretics are compounds that either compete with aldosterone for intracellular cytoplasmic receptor sites or directly block sodium channels, specifically epithelial sodium channels (ENaC). Examples of potassium sparing diuretics include, but are not limited to, amiloride, spironolactone, eplerenone, triamterene, potassium canrenoate.

Other diuretics contemplated for use also include, but are not limited to, caffeine, theophylline, theobromine, tolvaptan, conivaptan, dopamine, and pamabrom.

In some embodiments, the diuretic is dichlorphenamide, amiloride, pamabrom, mannitol, acetazolamide, methazolamide, spironolactone, triamterene, or the pharmaceutically acceptable salt or hydrate thereof. In some embodiments, the diuretic is dichlorphenamide. In some embodiments, the diuretic is amiloride. In some embodiments, the diuretic is pamabrom. In some embodiments, the diuretic is mannitol. In some embodiments, the diuretic is acetazolamide. In some embodiments, the diuretic is methazolamide. In some embodiments, the diuretic is spironolactone. In some embodiments, the diuretic is triamterene.

### Carbonic Anhydrase Inhibitors

Carbonic anhydrase inhibitors as used herein are compounds that are inhibitors of carbonic anhydrase. In some embodiments, the carbonic anhydrase inhibitor increases the excretion of bicarbonate with accompanying sodium, potassium and water, which results in an increased flow of alkaline urine. In some embodiments, the carbonic anhydrase inhibitor inhibits the transport of bicarbonate into the interstitium from the proximal convoluted tubule, which leads to less sodium being reabsorbed and provides greater sodium, bicarbonate and water loss in the urine. Examples of such compounds include but are not limited to acetazolamide, dichlorphenamide, and methazolamide.

### Calcium Channel Blockers

The present disclosure provides compositions comprising a calcium channel blocker. As used herein, calcium channel blockers are compounds that promote vasodilator activity by reducing calcium influx into vascular smooth muscle cells. In some embodiments, the calcium channel blocker is amlodipine, nifedipine, diltiazem, nimodipine, verapamil, isradipine, felodipine, nicardipine, nisoldipine, clevidipine, dihydropyridine, lercanidipine, nitrendipine, cilnidipine, manidipine, mibefradil, bepridil, barnidipine, nilvadipine, gallopamil, lidoflazine, aranidipine, dotarizine, diproteverine, or the pharmaceutically acceptable salt or hydrate thereof. In some embodiments, the calcium channel blocker is amlodipine, nifedipine, diltiazem, nimodipine, verapamil, isradipine, felodipine, nicardipine, nisoldipine, clevidipine or the pharmaceutically acceptable salt or hydrate thereof. In some embodiments, the calcium channel blocker is amlodipine or the pharmaceutically acceptable salt thereof. In some embodiments, the calcium channel blocker is amlodipine besylate. In some embodiments, the calcium channel blocker is nifedipine. In some embodiments, the calcium channel blocker is diltiazem. In some embodiments, the calcium channel blocker is nimodipine. In some embodiments, the calcium channel blocker is verapamil. In some embodiments, the calcium channel blocker is isradipine. In some embodiments, the calcium channel blocker is felodipine. In some embodiments, the calcium channel blocker is nicardipine. In some embodiments, the calcium channel blocker is nisoldipine. In some embodiments, the calcium channel blocker is clevidipine.

### Lowest Hypertension Therapeutic Dose

As used herein, the lowest hypertension therapeutic dose (LHTD) refers to the lowest strength dose for the single agent for hypertension approved by the US Food and Drug Administration and is not marked as “discontinued” by the Orange Book database (http://www.accessdata.fda.gov/scripts/cder/ob/) as of the filing date of this application. The lowest hypertension therapeutic dose does not include the lowest manufactured dose for cases wherein the lowest hypertension therapeutic dose is not the same as the lowest manufactured dose. Furthermore, the lowest hypertension therapeutic dose does not include the dose as recommended by a physician for cases wherein the lowest hypertension therapeutic dose is not the same dose as recommended by a physician. Furthermore, the lowest hypertension dose of the angiotensin II receptor blocker, diuretic, or calcium channel blocker described herein refers to the dose of the form of angiotensin II receptor blocker, diuretic, or calcium channel blocker approved for use by the US Food and Drug Administration, which includes the free base, pharmaceutically acceptable salt or hydrate thereof.

In some embodiments, the dose of the angiotensin II receptor blocker is from about 40% to about 80% of the lowest hypertension therapeutic dose. In some embodiments, the dose of the angiotensin II receptor blocker is from about 40% to about 70% of the lowest hypertension therapeutic dose. In some embodiments, the dose of the angiotensin II receptor blocker is from about 40% to about 60% of the lowest hypertension therapeutic dose. In some embodiments, the dose of the angiotensin II receptor blocker is from about 40% to about 50% of the lowest hypertension therapeutic dose. In some embodiments, the dose of the angiotensin II receptor blocker is from about 45% to about 55% of the lowest hypertension therapeutic dose. In some embodiments, the dose of the angiotensin II receptor blocker is from about 50% to about 80% of the lowest hypertension therapeutic dose. In some embodiments, the dose of the angiotensin II receptor blocker is from about 50% to about 70% of the lowest hypertension therapeutic dose. In some embodiments, the dose of the angiotensin II receptor blocker is from about 50% to about 60% of the lowest hypertension therapeutic dose. In some embodiments, the dose of the angiotensin II receptor blocker is from about 60% to about 80% of the lowest hypertension therapeutic dose. In some embodiments, the dose of the angiotensin II receptor blocker is from about 60% to about 70% of the lowest hypertension therapeutic dose. In some embodiments, the dose of the angiotensin II receptor blocker is from about 70% to about 80% of the lowest hypertension therapeutic dose.

In some embodiments, the dose of the angiotensin II receptor blocker is about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, or about 80% of the lowest hypertension therapeutic dose. In some embodiments, the dose of the angiotensin II receptor blocker is about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, or about 60% of the lowest hypertension therapeutic dose. In some embodiments, the dose of the angiotensin II receptor blocker is about 45%, about 46%, about 47%, about 48%, about 49%, about 50%, about 51%, about 52%, about 53%, about 54%, or about 55% of the lowest hypertension therapeutic dose. In some embodiments, the dose of the angiotensin II receptor blocker is about 50% of the lowest hypertension therapeutic dose. In some embodiments, the dose of the angiotensin II receptor blocker is about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, or about 80% of the lowest hypertension therapeutic dose. In some embodiments, the dose of the angiotensin II receptor blocker is about 61%, about 62%, about 63%, about 64%, about 65%, about 66%, about 67%, about 68%, about 69%, about 70%, or about 71% of the lowest hypertension therapeutic dose. In some embodiments, the dose of the angiotensin II receptor blocker is about 66% of the lowest hypertension therapeutic dose.

In some embodiments, the dose of the diuretic is from about 40% to about 80% of the lowest hypertension therapeutic dose. In some embodiments, the dose of the diuretic is from about 40% to about 70% of the lowest hypertension therapeutic dose. In some embodiments, the dose of the diuretic is from about 40% to about 60% of the lowest hypertension therapeutic dose. In some embodiments, the dose of the diuretic is from about 40% to about 50% of the lowest hypertension therapeutic dose. In some embodiments, the dose of the diuretic is from about 45% to about 55% of the lowest hypertension therapeutic dose. In some embodiments, the dose of the diuretic is from about 50% to about 80% of the lowest hypertension therapeutic dose. In some embodiments, the dose of the diuretic is from about 50% to about 70% of the lowest hypertension therapeutic dose. In some embodiments, the dose of the diuretic is from about 50% to about 60% of the lowest hypertension therapeutic dose. In some embodiments, the dose of the diuretic is from about 60% to about 80% of the lowest hypertension therapeutic dose. In some embodiments, the dose of the diuretic is from about 60% to about 70% of the lowest hypertension therapeutic dose. In some embodiments, the dose of the diuretic is from about 70% to about 80% of the lowest hypertension therapeutic dose.

In some embodiments, the dose of the diuretic is about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, or about 80% of the lowest hypertension therapeutic dose. In some embodiments, the dose of the diuretic is about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, or about 60% of the lowest hypertension therapeutic dose. In some embodiments, the dose of the diuretic is about 45%, about 46%, about 47%, about 48%, about 49%, about 50%, about 51%, about 52%, about 53%, about 54%, or about 55% of the lowest hypertension therapeutic dose. In some embodiments, the dose of the diuretic is about 50% of the lowest hypertension therapeutic dose. In some embodiments, the dose of the diuretic is about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, or about 80% of the lowest hypertension therapeutic dose. In some embodiments, the dose of the diuretic is about 61%, about 62%, about 63%, about 64%, about 65%, about 66%, about 67%, about 68%, about 69%, about 70%, or about 71% of the lowest hypertension therapeutic dose. In some embodiments, the dose of the diuretic is about 66% of the lowest hypertension therapeutic dose.

In some embodiments, the dose of the thiazide diuretic is from about 40% to about 80% of the lowest hypertension therapeutic dose. In some embodiments, the dose of the thiazide diuretic is from about 40% to about 70% of the lowest hypertension therapeutic dose. In some embodiments, the dose of the thiazide diuretic is from about 40% to about 60% of the lowest hypertension therapeutic dose. In some embodiments, the dose of the thiazide diuretic is from about 40% to about 50% of the lowest hypertension therapeutic dose. In some embodiments, the dose of the thiazide diuretic is from about 45% to about 55% of the lowest hypertension therapeutic dose. In some embodiments, the dose of the thiazide diuretic is from about 50% to about 80% of the lowest hypertension therapeutic dose. In some embodiments, the dose of the thiazide diuretic is from about 50% to about 70% of the lowest hypertension therapeutic dose. In some embodiments, the dose of the thiazide diuretic is from about 50% to about 60% of the lowest hypertension therapeutic dose. In some embodiments, the dose of the thiazide diuretic is from about 60% to about 80% of the lowest hypertension therapeutic dose. In some embodiments, the dose of the thiazide diuretic is from about 60% to about 70% of the lowest hypertension therapeutic dose. In some embodiments, the dose of the thiazide diuretic is from about 70% to about 80% of the lowest hypertension therapeutic dose.

In some embodiments, the dose of the thiazide diuretic is about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, or about 80% of the lowest hypertension therapeutic dose. In some embodiments, the dose of the thiazide diuretic is about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, or about 60% of the lowest hypertension therapeutic dose. In some embodiments, the dose of the thiazide diuretic is about 45%, about 46%, about 47%, about 48%, about 49%, about 50%, about 51%, about 52%, about 53%, about 54%, or about 55% of the lowest hypertension therapeutic dose. In some embodiments, the dose of the thiazide diuretic is about 50% of the lowest hypertension therapeutic dose. In some embodiments, the dose of the thiazide diuretic is about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, or about 80% of the lowest hypertension therapeutic dose. In some embodiments, the dose of the thiazide diuretic is about 61%, about 62%, about 63%, about 64%, about 65%, about 66%, about 67%, about 68%, about 69%, about 70%, or about 71% of the lowest hypertension therapeutic dose. In some embodiments, the dose of the thiazide diuretic is about 66% of the lowest hypertension therapeutic dose.

In some embodiments, the dose of the thiazide-like diuretic is from about 40% to about 80% of the lowest hypertension therapeutic dose. In some embodiments, the dose of the thiazide-like diuretic is from about 40% to about 70% of the lowest hypertension therapeutic dose. In some embodiments, the dose of the thiazide-like diuretic is from about 40% to about 60% of the lowest hypertension therapeutic dose. In some embodiments, the dose of the thiazide-like diuretic is from about 40% to about 50% of the lowest hypertension therapeutic dose. In some embodiments, the dose of the thiazide-like diuretic is from about 45% to about 55% of the lowest hypertension therapeutic dose. In some embodiments, the dose of the thiazide-like diuretic is from about 50% to about 80% of the lowest hypertension therapeutic dose. In some embodiments, the dose of the thiazide-like diuretic is from about 50% to about 70% of the lowest hypertension therapeutic dose. In some embodiments, the dose of the thiazide-like diuretic is from about 50% to about 60% of the lowest hypertension therapeutic dose. In some embodiments, the dose of the thiazide-like diuretic is from about 60% to about 80% of the lowest hypertension therapeutic dose. In some embodiments, the dose of the thiazide-like diuretic is from about 60% to about 70% of the lowest hypertension therapeutic dose. In some embodiments, the dose of the thiazide-like diuretic is from about 70% to about 80% of the lowest hypertension therapeutic dose.

In some embodiments, the dose of the thiazide-like diuretic is about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, or about 80% of the lowest hypertension therapeutic dose. In some embodiments, the dose of the thiazide-like diuretic is about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, or about 60% of the lowest hypertension therapeutic dose. In some embodiments, the dose of the thiazide-like diuretic is about 45%, about 46%, about 47%, about 48%, about 49%, about 50%, about 51%, about 52%, about 53%, about 54%, or about 55% of the lowest hypertension therapeutic dose. In some embodiments, the dose of the thiazide-like diuretic is about 50% of the lowest hypertension therapeutic dose. In some embodiments, the dose of the thiazide-like diuretic is about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, or about 80% of the lowest hypertension therapeutic dose. In some embodiments, the dose of the thiazide-like diuretic is about 61%, about 62%, about 63%, about 64%, about 65%, about 66%, about 67%, about 68%, about 69%, about 70%, or about 71% of the lowest hypertension therapeutic dose. In some embodiments, the dose of the thiazide-like diuretic is about 66% of the lowest hypertension therapeutic dose.

In some embodiments, the dose of the loop diuretic is from about 40% to about 80% of the lowest hypertension therapeutic dose. In some embodiments, the dose of the loop diuretic is from about 40% to about 70% of the lowest hypertension therapeutic dose. In some embodiments, the dose of the loop diuretic is from about 40% to about 60% of the lowest hypertension therapeutic dose. In some embodiments, the dose of the loop diuretic is from about 40% to about 50% of the lowest hypertension therapeutic dose. In some embodiments, the dose of the loop diuretic is from about 45% to about 55% of the lowest hypertension therapeutic dose. In some embodiments, the dose of the loop diuretic is from about 50% to about 80% of the lowest hypertension therapeutic dose. In some embodiments, the dose of the loop diuretic is from about 50% to about 70% of the lowest hypertension therapeutic dose. In some embodiments, the dose of the loop diuretic is from about 50% to about 60% of the lowest hypertension therapeutic dose. In some embodiments, the dose of the loop diuretic is from about 60% to about 80% of the lowest hypertension therapeutic dose. In some embodiments, the dose of the loop diuretic is from about 60% to about 70% of the lowest hypertension therapeutic dose. In some embodiments, the dose of the loop diuretic is from about 70% to about 80% of the lowest hypertension therapeutic dose.

In some embodiments, the dose of the loop diuretic is about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, or about 80% of the lowest hypertension therapeutic dose. In some embodiments, the dose of the loop diuretic is about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, or about 60% of the lowest hypertension therapeutic dose. In some embodiments, the dose of the loop diuretic is about 45%, about 46%, about 47%, about 48%, about 49%, about 50%, about 51%, about 52%, about 53%, about 54%, or about 55% of the lowest hypertension therapeutic dose. In some embodiments, the dose of the loop diuretic is about 50% of the lowest hypertension therapeutic dose. In some embodiments, the dose of the loop diuretic is about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, or about 80% of the lowest hypertension therapeutic dose. In some embodiments, the dose of the loop diuretic is about 61%, about 62%, about 63%, about 64%, about 65%, about 66%, about 67%, about 68%, about 69%, about 70%, or about 71% of the lowest hypertension therapeutic dose. In some embodiments, the dose of the loop diuretic is about 66% of the lowest hypertension therapeutic dose.

In some embodiments, the dose of the calcium channel blocker is from about 40% to about 80% of the lowest hypertension therapeutic dose. In some embodiments, the dose of the calcium channel blocker is from about 40% to about 70% of the lowest hypertension therapeutic dose. In some embodiments, the dose of the calcium channel blocker is from about 40% to about 60% of the lowest hypertension therapeutic dose. In some embodiments, the dose of the calcium channel blocker is from about 40% to about 50% of the lowest hypertension therapeutic dose. In some embodiments, the dose of the calcium channel blocker is from about 45% to about 55% of the lowest hypertension therapeutic dose. In some embodiments, the dose of the calcium channel blocker is from about 50% to about 80% of the lowest hypertension therapeutic dose. In some embodiments, the dose of the calcium channel blocker is from about 50% to about 70% of the lowest hypertension therapeutic dose. In some embodiments, the dose of the calcium channel blocker is from about 50% to about 60% of the lowest hypertension therapeutic dose. In some embodiments, the dose of the calcium channel blocker is from about 60% to about 80% of the lowest hypertension therapeutic dose. In some embodiments, the dose of the calcium channel blocker is from about 60% to about 70% of the lowest hypertension therapeutic dose. In some embodiments, the dose of the calcium channel blocker is from about 70% to about 80% of the lowest hypertension therapeutic dose.

In some embodiments, the dose of the calcium channel blocker is about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, or about 80% of the lowest hypertension therapeutic dose. In some embodiments, the dose of the calcium channel blocker is about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, or about 60% of the lowest hypertension therapeutic dose. In some embodiments, the dose of the calcium channel blocker is about 45%, about 46%, about 47%, about 48%, about 49%, about 50%, about 51%, about 52%, about 53%, about 54%, or about 55% of the lowest hypertension therapeutic dose. In some embodiments, the dose of the calcium channel blocker is about 50% of the lowest hypertension therapeutic dose. In some embodiments, the dose of the calcium channel blocker is about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, or about 80% of the lowest hypertension therapeutic dose. In some embodiments, the dose of the calcium channel blocker is about 61%, about 62%, about 63%, about 64%, about 65%, about 66%, about 67%, about 68%, about 69%, about 70%, or about 71% of the lowest hypertension therapeutic dose. In some embodiments, the dose of the calcium channel blocker is about 66% of the lowest hypertension therapeutic dose.

In some embodiments, the dose of telmisartan is about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, about 25 mg, about 26 mg, about 27 mg, about 28 mg, about 29 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, or about 50 mg. In some embodiments, the dose of telmisartan is from about 5 mg to about 10 mg, from about 5 mg to about 15 mg, from about 5 mg to about 20 mg, from about 5 mg to about 25 mg, from about 5 mg to about 30 mg, from about 5 mg to about 35 mg, from about 5 mg to about 40 mg, from about 5 mg to about 45 mg, from about 5 mg to about 50 mg, from about 8 mg to about 10 mg, from about 8 mg to about 12 mg, from about 8 mg to about 15 mg, from about 8 mg to about 20 mg, from about 8 mg to about 25 mg, from about 8 mg to about 30 mg, from about 8 mg to about 35 mg, from about 8 mg to about 40 mg, about 8 mg to about 45 mg, about 8 mg to about 50 mg, from about 10 mg to about 12 mg, from about 10 mg to about 15 mg, from about 10 mg to about 20 mg, from about 10 mg to about 25 mg, from about 10 mg to about 30 mg, from about 10 mg to about 35 mg, from about 10 mg to about 40 mg, from about 10 mg to about 45 mg, from about 10 mg to about 50 mg, from about 12 mg to about 15 mg, from about 12 mg to about 20 mg, from about 12 mg to about 25 mg, from about 12 mg to about 30 mg, from about 12 mg to about 35 mg, from about 12 mg to about 40 mg, from about 15 mg to about 20 mg, from about 15 mg to about 25 mg, from about 15 mg to about 30 mg, from about 15 mg to about 35 mg, from about 15 mg to about 40 mg, from about 15 mg to about 45 mg, from about 15 mg to about 50 mg, from about 20 mg to about 25 mg, from about 20 mg to about 30 mg, from about 20 mg to about 35 mg, from about 20 mg to about 40 mg, from about 20 mg to about 45 mg, from about 20 mg to about 50 mg, from about 25 mg to about 30 mg, from about 25 mg to about 35 mg, from about 25 mg to about 40 mg, from about 25 mg to about 45 mg, from about 25 mg to about 50 mg, from about 30 mg to about 35 mg, from about 30 mg to about 40 mg, from about 35 mg to about 40 mg, from about 30 mg to about 45 mg, from about 30 mg to about 50 mg, from about 35 mg to about 40 mg, from about 30 mg to about 45 mg, from about 30 mg to about 50 mg, from about 40 mg to about 45 mg, from about 40 mg to about 50 mg, or from about 45 mg to about 50 mg.

In some embodiments, the dose of indapamide is about 0.1 mg, about 0.2 mg, about 0.3 mg, about 0.4 mg, about 0.5 mg, about 0.6 mg, about 0.7 mg, about 0.8 mg, about 0.9 mg, about 1 mg, about 1.1 mg, about 1.2 mg, about 1.3 mg, about 1.4 mg, about 1.5 mg, about 1.6 mg, about 1.7 mg, about 1.8 mg, about 1.9 mg, about 2 mg, about 2.1 mg, about 2.2 mg, about 2.3 mg, about 2.4 mg, about 2.5 mg, about 3 mg, about 3.1 mg, about 3.2 mg, about 3.3 mg, about 3.4 mg, about 3.5 mg, about 3.6 mg, about 3.7 mg, about 3.8 mg, about 3.9 mg, about 4 mg, about 4.1 mg, about 4.2 mg, about 4.3 mg, about 4.4 mg, about 4.5, mg, about 4.6 mg, about 4.7 mg, about 4.8 mg, about 4.9 mg, or about 5 mg. In some embodiments, the dose of indapamide is from about 0.1 mg to about 0.5 mg, from about 0.1 mg to about 1 mg, from about 0.1 mg to about 1.5 mg, from about 0.1 mg to about 2 mg, from about 0.1 mg to about 2.5 mg, from about 0.5 mg to about 1 mg, from about 0.5 mg to about 1.5 mg, from about 0.5 mg to about 2 mg, from about 0.5 mg to about 2.5 mg, from about 0.5 mg to about 3 mg, from about 0.5 mg to about 4 mg, about 0.5 mg to about 5 mg, from about 1 mg to about 1.5 mg, from about 1 mg to about 2 mg, from about 1 mg to about 2.5 mg, from about 1 mg to about 3 mg, from about 1 mg to about 4 mg, from about 1 mg to about 5 mg, from about 1.5 mg to about 2 mg, from about 1.5 mg to about 2.5 mg, from about 1.5 mg to about 3 mg, about 1.5 mg to about 4 mg, about 1.5 mg to about 5 mg, from about 2 mg to about 2.5 mg, from about 2 mg to about 3 mg, from about 2 mg to about 4 mg, or from about 2 mg to about 5 mg.

A dose of a therapeutic agent as claimed herein can be expressed as an amount of its salt or an equivalent amount of the base. For instance, a dose of amlodipine besylate as claimed herein can be expressed as an amount of an amlodipine salt, or as an amount of amlodipine. In some embodiments, the amount of amlodipine besylate is expressed as an amount of the base (amlodipine). In some embodiments, an amount of amlodipine can be referred to as an amount of amlodipine, an amount of amlodipine base, an amount of amlodipine free base, or variants thereof. In some embodiments, the amlodipine salt is amlodipine besylate. 1 mg of amlodipine is equivalent to about 1.38 mg of amlodipine besylate. In some embodiments, the dose of amlodipine besylate is about EQ 1.25 mg base. 1.25 mg amlodipine is equivalent to about 1.73 mg amlodipine besylate. In some embodiments, the dose of amlodipine besylate is about EQ 2.5 mg base. 2.5 mg amlodipine is equivalent to about 3.47 mg amlodipine besylate. In some embodiments, the dose of amlodipine besylate is about EQ 5 mg base. 5 mg amlodipine is equivalent to about 6.94 mg amlodipine besylate.

In some embodiments, the dose of amlodipine besylate is about 0.2 mg, about 0.4 mg, about 0.6 mg, about 0.8 mg, about 1 mg, about 1.2 mg, about 1.25 mg, about 1.4 mg, about 1.6 mg, about 1.7 mg, about 1.73 mg, about 1.8 mg, about 2 mg, about 2.2 mg, about 2.4 mg, about 2.5 mg, about 2.6 mg, about 2.8 mg, about 3 mg, about 3.2 mg, about 3.4 mg, about 3.47 mg, about 3.6 mg, about 3.8 mg, about 4 mg, about 4.2 mg, about 4.4 mg, about 4.6 mg, about 4.8 mg, about 5 mg, about 5.2 mg, about 5.4 mg, about 6.5 mg, about 5.8 mg, about 6 mg, about 6.2 mg, about 6.4 mg, about 6.6 mg, about 6.8 mg, about 6.9 mg, about 6.94 mg, about 7 mg, about 7.2 mg, about 7.4 mg, about 7.6 mg, about 7.8 mg, or about 8 mg. In some embodiments, the dose of amlodipine besylate is from about 0.2 mg to about 1 mg, from about 0.2 mg to about 1.8 mg, from about 0.2 mg to about 2.6 mg, from about 0.2 mg to about 3.4 mg, from about 0.2 mg to about 4.2 mg, from about 0.2 mg to about 5 mg, from about 0.2 mg to about 6 mg, from about 0.2 mg to about 7 mg, from about 0.2 mg to about 8 mg, from about 1 mg to about 1.8 mg, from about 1 mg to about 2.6 mg, from about 1 mg to about 3.4 mg, from about 1 mg to about 4.2 mg, from about 1 mg to about 5 mg, from about 1 mg to about 6 mg, from about 1 mg to about 7 mg, from about 1 mg to about 8 mg, from about 1.8 mg to about 2.6 mg, from about 1.8 mg to about 3.4 mg, from about 1.8 mg to about 4.2 mg, from about 1.8 mg to about 5 mg, from about 1.8 mg to about 6 mg, from about 1.8 mg to about 7 mg, from about 1.8 mg to about 8 mg, from about 2.6 mg to about 3.4 mg, from about 2.6 mg to about 4.2 mg, from about 2.6 mg to about 5 mg, from about 2.6 mg to about 6 mg, from about 2.6 mg to about 7 mg, from about 2.6 mg to about 8 mg, from about 3.4 mg to about 4.2 mg, from about 3.4 mg to about 5 mg, from about 3.4 mg to about 6 mg, from about 3.4 mg to about 7 mg, from about 3.4 mg to about 8 mg, from about 4.2 mg to about 5 mg, from about 4.2 mg to about 6 mg, from about 4.2 mg to about 7 mg, or from about 4.2 mg to about 8 mg. In some embodiments, the dose of amlodipine besylate is from about 1.7 mg to about 1.8 mg, from about 3.4 mg to about 3.5 mg, or from about 6.9 mg to about 7.0 mg. In some embodiments, the dose of amlodipine besylate is from about 1.4 mg to about 1.6 mg, from about 2.4 mg to about 2.6 mg, or from about 4.9 mg to about 5.1 mg.

In some embodiments, the dose of amlodipine besylate is about EQ 0.2 mg base, about EQ 0.4 mg base, about EQ 0.6 mg base, about EQ 0.8 mg base, about EQ 1 mg base, about EQ 1.2 mg base, about EQ 1.25 mg base, about EQ 1.4 mg base, about EQ 1.6 mg base, about EQ 1.7 mg base, about EQ 1.73 mg base, about EQ 1.8 mg base, about EQ 2 mg base, about EQ 2.2 mg base, about EQ 2.4 mg base, about EQ 2.5 mg base, about EQ 2.6 mg base, about EQ 2.8 mg base, about EQ 3 mg base, about EQ 3.2 mg base, about EQ 3.4 mg base, about EQ 3.47 mg base, about EQ 3.6 mg base, about EQ 3.8 mg base, about EQ 4 mg base, about EQ 4.2 mg base, about EQ 4.4 mg base, about EQ 4.6 mg base, about EQ 4.8 mg base, about EQ 5 mg base, about EQ 5.2 mg base, about EQ 5.4 mg base, about EQ 6.5 mg base, about EQ 5.8 mg base, about EQ 6 mg base, about EQ 6.2 mg base, about EQ 6.4 mg base, about EQ 6.6 mg base, about EQ 6.8 mg base, about EQ 6.9 mg base, about EQ 6.94 mg base, about EQ 7 mg base, about EQ 7.2 mg base, about EQ 7.4 mg base, about EQ 7.6 mg base, about EQ 7.8 mg base, or about EQ 8 mg base. In some embodiments, the dose of amlodipine besylate is from about EQ 0.2 mg base to about EQ 1 mg base, from about EQ 0.2 mg base to about EQ 1.8 mg base, from about EQ 0.2 mg base to about EQ 2.6 mg base, from about EQ 0.2 mg base to about EQ 3.4 mg base, from about EQ 0.2 mg base to about EQ 4.2 mg base, from about EQ 0.2 mg base to about EQ 5 mg base, from about EQ 0.2 mg base to about EQ 6 mg base, from about EQ 0.2 mg base to about EQ 7 mg base, from about EQ 0.2 mg base to about EQ 8 mg base, from about EQ 1 mg base to about EQ 1.8 mg base, from about EQ 1 mg base to about EQ 2.6 mg base, from about EQ 1 mg base to about EQ 3.4 mg base, from about EQ 1 mg base to about EQ 4.2 mg base, from about EQ 1 mg base to about EQ 5 mg base, from about EQ 1 mg base to about EQ 6 mg base, from about EQ 1 mg base to about EQ 7 mg base, from about EQ 1 mg base to about EQ 8 mg base, from about EQ 1.8 mg base to about EQ 2.6 mg base, from about EQ 1.8 mg base to about EQ 3.4 mg base, from about EQ 1.8 mg base to about EQ 4.2 mg base, from about EQ 1.8 mg base to about EQ 5 mg base, from about EQ 1.8 mg base to about EQ 6 mg base, from about EQ 1.8 mg base to about EQ 7 mg base, from about EQ 1.8 mg base to about EQ 8 mg base, from about EQ 2.6 mg base to about EQ 3.4 mg base, from about EQ 2.6 mg base to about EQ 4.2 mg base, from about EQ 2.6 mg base to about EQ 5 mg base, from about EQ 2.6 mg base to about EQ 6 mg base, from about EQ 2.6 mg base to about EQ 7 mg base, from about EQ 2.6 mg base to about EQ 8 mg base, from about EQ 3.4 mg base to about EQ 4.2 mg base, from about EQ 3.4 mg base to about EQ 5 mg base, from about EQ 3.4 mg base to about EQ 6 mg base, from about EQ 3.4 mg base to about EQ 7 mg base, from about EQ 3.4 mg base to about EQ 8 mg base, from about EQ 4.2 mg base to about EQ 5 mg base, from about EQ 4.2 mg base to about EQ 6 mg base, from about EQ 4.2 mg base to about EQ 7 mg base, or from about EQ 4.2 mg base to about EQ 8 mg base. In some embodiments, the dose of amlodipine besylate is from about EQ 1.7 mg base to about EQ 1.8 mg base, from about EQ 3.4 mg base to about EQ 3.5 mg base, or from about EQ 6.9 mg base to about EQ 7.0 mg base. In some embodiments, the dose of amlodipine besylate is from about EQ 1.4 mg base to about EQ 1.6 mg base, from about EQ 2.4 mg base to about EQ 2.6 mg base, or from about EQ 4.9 mg base to about EQ 5.1 mg base.

In some embodiments, the lowest hypertension therapeutic dose (LHTD) and the corresponding proposed dose and proposed dose range for the following compounds are as described in the following table:

TABLE 2

| | Lowest | | |
|---|---|---|---|
| | Hypertension | Proposed | Proposed Dose |
| | Therapeutic | Dose (mg) | Range (mg) |
| Agent | Dose (mg) | (% LHTD) | (% LHTD) |
| Amlodipine | EQ 2.5 mg | EQ 1.25 mg | EQ 1 mg base - EQ |
| besylate | base | base (50% of | 1.5 mg base (40%- |
| | | LHTD) or | 60% of LHTD) or |
| | | EQ 1.65 mg | EQ 1.5 mg base - |
| | | base (66% of | EQ 2 mg base |
| | | LHTD) | (60%-80% of |
| | | | LHTD) |
| Chlorthalidone | 25 | 12.5 (50% of | 10-15 (40%-60% of |
| | | LHTD) or | LHTD) or |
| | | 16.5 (66% of | 15-20 (60%-80% of |
| | | LHTD) | LHTD) |
| Hydrochlorothiazide | 12.5 | 6.25 (50% of | 5-7.5 (40%-60% of |
| | | LHTD) or | LHTD) or |
| | | 8.25 (66% of | 7.5-10 (60%-80% |
| | | LHTD) | of LHTD) |
| Indapamide | 1.25 | 0.625 (50% | 0.5-0.75 (40%-60% |
| | | of LHTD) or | of LHTD) or |
| | | 0.825 (66% | 0.75-1.0 (60%-80% |
| | | of LHTD) | of LHTD) |
| Irbesartan | 75 | 37.5 (50% of | 30-45 (40%-60% of |
| | | LHTD) or | LHTD) or |
| | | 49.5 (66% of | 45-60 (60%-80% of |
| | | LHTD) | LHTD) |
| Telmisartan | 20 | 10 (50% of | 8-12 (40%-60% of |
| | | LHTD) or | LHTD) or |
| | | 13.2 (66% of | 12-16 (60%-80% of |
| | | LHTD) | LHTD) |

In some embodiments, the pharmaceutical composition comprises: (a) irbesartan as an angiotensin II receptor blocker; (b) hydrochlorothiazide as a thiazide diuretic; and (c) amlodipine besylate as a calcium channel blocker. In some embodiments, the dose of irbesartan is about 30 mg to about 45 mg, the dose of hydrochlorothiazide is about 5 mg to about 7.5 mg, and the dose of amlodipine besylate is about EQ 1 mg base to about EQ 1.5 mg base. In some embodiments, the dose of irbesartan is about 37.5 mg, the dose of hydrochlorothiazide is about 6.25 mg, and the dose of amlodipine besylate is about EQ 1.25 mg base.

In some embodiments, the pharmaceutical composition comprises: (a) telmisartan as an angiotensin II receptor blocker; (b) hydrochlorothiazide as a thiazide diuretic; and (c) amlodipine besylate as a calcium channel blocker. In some embodiments, the dose of telmisartan is about 8 mg to about 12 mg, the dose of hydrochlorothiazide is about 5 mg to about 7.5 mg, and the dose of amlodipine besylate is about EQ 1 mg base to about EQ 1.5 mg base. In some embodiments, the dose of telmisartan is about 10 mg, the dose of hydrochlorothiazide is about 6.25 mg, and the dose of amlodipine besylate is about EQ 1.25 mg base.

In some embodiments, the pharmaceutical composition comprises: (a) irbesartan as an angiotensin II receptor blocker; (b) indapamide as a thiazide-like diuretic; and (c) amlodipine besylate as a calcium channel blocker. In some embodiments, the dose of irbesartan is about 30 mg to about 45 mg, the dose of indapamide is about 0.5 mg to about 0.75 mg, and the dose of amlodipine besylate is about EQ 1 mg base to about EQ 1.5 mg base. In some embodiments, the dose of irbesartan is about 37.5 mg, the dose of indapamide is about 0.625 mg, and the dose of amlodipine besylate is about EQ 1.25 mg base.

In some embodiments, the pharmaceutical composition comprises: (a) telmisartan as an angiotensin II receptor blocker; (b) indapamide as a thiazide-like diuretic; (c) amlodipine besylate as a calcium channel blocker. In some embodiments, the dose of telmisartan is about 8 mg to about 12 mg, the dose of indapamide is about 0.5 mg to about 0.75 mg, and the dose of amlodipine besylate is about EQ 1 mg base to about EQ 1.5 mg base. In some embodiments, dose of telmisartan is about 10 mg, the dose of indapamide is about 0.625 mg, and the dose of amlodipine besylate is about EQ 1.25 mg base.

In some embodiments, the pharmaceutical composition comprises: (a) telmisartan as an angiotensin II receptor blocker; (b) chlorthalidone as a thiazide-like diuretic; and (c) amlodipine besylate as a calcium channel blocker. In some embodiments, the dose of telmisartan is about 8 mg to about 12 mg, the dose of chlorthalidone is about 10 mg to about 15 mg, and the dose of amlodipine besylate is about EQ 1 mg base to about EQ 1.5 mg base. In some embodiments, dose of telmisartan is about 10 mg, the dose of chlorthalidone is about 12.5 mg, and the dose of amlodipine besylate is about EQ 1.25 mg base.

In some embodiments, the pharmaceutical composition comprises: (a) irbesartan as an angiotensin II receptor blocker; (b) chlorthalidone as a thiazide-like diuretic; and (c) amlodipine besylate as a calcium channel blocker. In some embodiments, the dose of irbesartan is about 30 mg to about 45 mg, the dose of chlorthalidone is about 10 mg to about 15 mg, and the dose of amlodipine besylate is about EQ 1 mg base to about EQ 1.5 mg base. In some embodiments, dose of irbesartan is about 37.5 mg, the dose of chlorthalidone is about 12.5 mg, and the dose of amlodipine besylate is about EQ 1.25 mg base.

In some embodiments, the pharmaceutical composition comprises: (a) irbesartan as an angiotensin II receptor blocker; (b) hydrochlorothiazide as a thiazide diuretic; and (c) amlodipine besylate as a calcium channel blocker. In some embodiments, the dose of irbesartan is about 45 mg to about 60 mg, the dose of hydrochlorothiazide is about 7.5 mg to about 10 mg, and the dose of amlodipine besylate is about EQ 1.5 mg base to about EQ 2 mg base. In some embodiments, the dose of irbesartan is about 49.5 mg, the dose of hydrochlorothiazide is about 8.25 mg, and the dose of amlodipine besylate is about EQ 1.65 mg base.

In some embodiments, the pharmaceutical composition comprises: (a) telmisartan as an angiotensin II receptor blocker; (b) hydrochlorothiazide as a thiazide diuretic; and (c) amlodipine besylate as a calcium channel blocker. In some embodiments, the dose of telmisartan is about 12 mg to about 16 mg, the dose of hydrochlorothiazide is about 7.5 mg to about 10 mg, and the dose of amlodipine besylate is about EQ 1.5 mg base to about EQ 2 mg base. In some embodiments, the dose of telmisartan is about 13.2 mg, the dose of hydrochlorothiazide is about 8.25 mg, and the dose of amlodipine besylate is about EQ 1.65 mg base.

In some embodiments, the pharmaceutical composition comprises: (a) irbesartan as an angiotensin II receptor blocker; (b) indapamide as a thiazide-like diuretic; and (c) amlodipine besylate as a calcium channel blocker. In some embodiments, the dose of irbesartan is about 45 mg to about 60 mg, the dose of indapamide is about 0.75 mg to about 1.0 mg, and the dose of amlodipine besylate is about EQ 1.5 mg base to about EQ 2 mg base. In some embodiments, the dose of irbesartan is about 49.5 mg, the dose of indapamide is about 0.825 mg, and the dose of amlodipine besylate is about EQ 1.65 mg base.

In some embodiments, the pharmaceutical composition comprises: (a) telmisartan as an angiotensin II receptor blocker; (b) indapamide as a thiazide-like diuretic; (c) amlodipine besylate as a calcium channel blocker. In some embodiments, the dose of telmisartan is about 12 mg to about 16 mg, the dose of indapamide is about 0.75 mg to about 1.0 mg, and the dose of amlodipine besylate is about EQ 1.5 mg base to about EQ 2 mg base. In some embodiments, dose of telmisartan is about 13.2 mg, the dose of indapamide is about 0.825 mg, and the dose of amlodipine besylate is about EQ 1.65 mg base.

In some embodiments, the pharmaceutical composition comprises: (a) telmisartan as an angiotensin II receptor blocker; (b) chlorthalidone as a thiazide-like diuretic; and (c) amlodipine besylate as a calcium channel blocker. In some embodiments, the dose of telmisartan is about 12 mg to about 16 mg, the dose of chlorthalidone is about 15 mg to about 20 mg, and the dose of amlodipine besylate is about EQ 1.5 mg base to about EQ 2 mg base. In some embodiments, dose of telmisartan is about 13.2 mg, the dose of chlorthalidone is about 16.5 mg, and the dose of amlodipine besylate is about EQ 1.65 mg base.

In some embodiments, the pharmaceutical composition comprises: (a) irbesartan as an angiotensin II receptor blocker; (b) chlorthalidone as a thiazide-like diuretic; and (c) amlodipine besylate as a calcium channel blocker. In some embodiments, the dose of irbesartan is about 45 mg to about 60 mg, the dose of chlorthalidone is about 15 mg to about 20 mg, and the dose of amlodipine besylate is about EQ 1.5 mg base to about EQ 2 mg base. In some embodiments, dose of irbesartan is about 49.5 mg, the dose of chlorthalidone is about 16.5 mg, and the dose of amlodipine besylate is about EQ 1.65 mg base.

In some embodiments, the lowest hypertension therapeutic dose (LHTD) and the corresponding proposed dose and proposed dose range for the following compounds are as described in the following table:

TABLE 3

| | Lowest | | |
|---|---|---|---|
| | Hypertension | Proposed | Proposed Dose |
| | Therapeutic | Dose (mg) | Range (mg) |
| Agent | Dose (mg) | (% LHTD) | (% LHTD) |
| Amlodipine | EQ 2.5 | EQ 2.5 mg | EQ 2 mg base - EQ |
| besylate | mg base | base (100% | 3.75 mg base (80%- |
| | | of LHTD) or | 150% of LHTD) or |
| | | EQ 5 mg | EQ 3.75 mg base - |
| | | base (200% | EQ 6.25 mg base |
| | | of LHTD) | (150%-250% of |
| | | | LHTD) |
| Chlorthalidone | 25 | 25 (100% of | 20-37.5 (80%- |
| | | LHTD) or | 150% of LHTD) or |
| | | 50 (200% of | 37.5-62.5 (150%- |
| | | LHTD) | 250% of LHTD) |
| Hydrochlorothiazide | 12.5 | 12.5 (100% | 10-18.75 (80%- |
| | | of LHTD) or | 150% of LHTD) or |
| | | 25 (200% of | 18.75-31.25 (150%- |
| | | LHTD) | 250% of LHTD) |
| Indapamide | 1.25 | 1.25 (100% | 1-1.875 (80%-150% |
| | | of LHTD) or | of LHTD) or |
| | | 2.5 (200% of | 1.875-3.125 (150%- |
| | | LHTD) | 250% of LHTD) |
| Irbesartan | 75 | 75 (100% of | 60-112.5 (80%- |
| | | LHTD) or | 150% of LHTD) or |
| | | 150 (200% of | 112.5-187.5 (150%- |
| | | LHTD) | 250% of LHTD) |
| Telmisartan | 20 | 20 (100% of | 16-30 (80%-150% |
| | | LHTD) or | of LHTD) or |
| | | 40 (200% of | 30-50 (150%-250% |
| | | LHTD) | of LHTD) |

In some embodiments, the dose of any one of the angiotensin II receptor blockers, the diuretic, and the calcium channel blocker is substituted with from about 80% to about 250% of the lowest hypertension therapeutic dose (LHTD) for the angiotensin II receptor blocker, the diuretic, or the calcium channel blocker. In some embodiments, the dose of the angiotensin II receptor blocker is substituted with from about 80% to about 250% of the lowest hypertension therapeutic dose (LHTD) for the angiotensin II receptor blocker. In some embodiments, the dose of the diuretic is substituted with from about 80% to about 250% of the lowest hypertension therapeutic dose (LHTD) for the diuretic. In some embodiments, the dose of the calcium channel blocker is substituted with from about 80% to about 250% of the lowest hypertension therapeutic dose (LHTD) for the calcium channel blocker. In some embodiments, the dose of any one of the angiotensin II receptor blockers, the diuretic, and the calcium channel blocker is substituted with from about 80% to about 150% of the lowest hypertension therapeutic dose (LHTD) for the angiotensin II receptor blocker, the diuretic, or the calcium channel blocker. In some embodiments, the dose of the angiotensin II receptor blocker is substituted with from about 80% to about 150% of the lowest hypertension therapeutic dose (LHTD) for the angiotensin II receptor blocker. In some embodiments, the dose of the angiotensin II receptor blocker is substituted with about 100% of the lowest hypertension therapeutic dose (LHTD) for the angiotensin II receptor blocker. In some embodiments, the dose of the diuretic is substituted with from about 80% to about 150% of the lowest hypertension therapeutic dose (LHTD) for the diuretic. In some embodiments, the dose of the diuretic is substituted with about 100% of the lowest hypertension therapeutic dose (LHTD) for the diuretic. In some embodiments, the dose of the calcium channel blocker is substituted with from about 80% to about 150% of the lowest hypertension therapeutic dose (LHTD) for the calcium channel blocker. In some embodiments, the dose of the calcium channel blocker is substituted with about 100% of the lowest hypertension therapeutic dose (LHTD) for the calcium channel blocker. In some embodiments, the dose of any one of the angiotensin II receptor blockers, the diuretic, and the calcium channel blocker is substituted with from about 150% to about 250% of the lowest hypertension therapeutic dose (LHTD) for the angiotensin II receptor blocker, the diuretic, or the calcium channel blocker. In some embodiments, the dose of the angiotensin II receptor blocker is substituted with from about 150% to about 250% of the lowest hypertension therapeutic dose (LHTD) for the angiotensin II receptor blocker. In some embodiments, the dose of the angiotensin II receptor blocker is substituted with about 200% of the lowest hypertension therapeutic dose (LHTD) for the angiotensin II receptor blocker. In some embodiments, the dose of the diuretic is substituted with from about 150% to about 250% of the lowest hypertension therapeutic dose (LHTD) for the diuretic. In some embodiments, the dose of the diuretic is substituted with about 200% of the lowest hypertension therapeutic dose (LHTD) for the diuretic.

In some embodiments, the dose of the calcium channel blocker is substituted with from about 150% to about 250% of the lowest hypertension therapeutic dose (LHTD) for the calcium channel blocker. In some embodiments, the dose of the calcium channel blocker is substituted with about 200% of the lowest hypertension therapeutic dose (LHTD) for the calcium channel blocker.

In some embodiments, the dose of any two of the angiotensin II receptor blockers, the diuretic, and the calcium channel blocker is substituted with from about 80% to about 250% of the lowest hypertension therapeutic dose (LHTD) for the angiotensin II receptor blocker, the diuretic, or the calcium channel blocker. In some embodiments, the dose of the angiotensin II receptor blocker is substituted with from about 80% to about 250% of the lowest hypertension therapeutic dose (LHTD) for the angiotensin II receptor blocker. In some embodiments, the dose of the diuretic is substituted with from about 80% to about 250% of the lowest hypertension therapeutic dose (LHTD) for the diuretic. In some embodiments, the dose of the calcium channel blocker is substituted with from about 80% to about 250% of the lowest hypertension therapeutic dose (LHTD) for the calcium channel blocker. In some embodiments, the dose of any two of the angiotensin II receptor blockers, the diuretic, and the calcium channel blocker is substituted with from about 80% to about 150% of the lowest hypertension therapeutic dose (LHTD) for the angiotensin II receptor blocker, the diuretic, or the calcium channel blocker. In some embodiments, the dose of the angiotensin II receptor blocker is substituted with from about 80% to about 150% of the lowest hypertension therapeutic dose (LHTD) for the angiotensin II receptor blocker. In some embodiments, the dose of the angiotensin II receptor blocker is substituted with about 100% of the lowest hypertension therapeutic dose (LHTD) for the angiotensin II receptor blocker. In some embodiments, the dose of the diuretic is substituted with from about 80% to about 150% of the lowest hypertension therapeutic dose (LHTD) for the diuretic. In some embodiments, the dose of the diuretic is substituted with about 100% of the lowest hypertension therapeutic dose (LHTD) for the diuretic. In some embodiments, the dose of the calcium channel blocker is substituted with from about 80% to about 150% of the lowest hypertension therapeutic dose (LHTD) for the calcium channel blocker. In some embodiments, the dose of the calcium channel blocker is substituted with about 100% of the lowest hypertension therapeutic dose (LHTD) for the calcium channel blocker. In some embodiments, the dose of any two of the angiotensin II receptor blockers, the diuretic, and the calcium channel blocker is substituted with from about 150% to about 250% of the lowest hypertension therapeutic dose (LHTD) for the angiotensin II receptor blocker, the diuretic, or the calcium channel blocker. In some embodiments, the dose of the angiotensin II receptor blocker is substituted with from about 150% to about 250% of the lowest hypertension therapeutic dose (LHTD) for the angiotensin II receptor blocker. In some embodiments, the dose of the angiotensin II receptor blocker is substituted with about 200% of the lowest hypertension therapeutic dose (LHTD) for the angiotensin II receptor blocker. In some embodiments, the dose of the diuretic is substituted with from about 150% to about 250% of the lowest hypertension therapeutic dose (LHTD) for the diuretic. In some embodiments, the dose of the diuretic is substituted with about 200% of the lowest hypertension therapeutic dose (LHTD) for the diuretic. In some embodiments, the dose of the calcium channel blocker is substituted with from about 150% to about 250% of the lowest hypertension therapeutic dose (LHTD) for the calcium channel blocker. In some embodiments, the dose of the calcium channel blocker is substituted with about 200% of the lowest hypertension therapeutic dose (LHTD) for the calcium channel blocker.

### Formulations

In some embodiments, the angiotensin II receptor blocker, the diuretic, and the calcium channel blocker are provided in one formulation. In some embodiments, the angiotensin II receptor blocker, the diuretic, and the calcium channel blocker each provided in a separate formulation. In some embodiments, two of the angiotensin II receptor blockers, the diuretic, and the calcium channel blocker are provided in one formulation. In some embodiments, the angiotensin II receptor and the diuretic are provided in one formulation. In some embodiments, the angiotensin II receptor blocker and the calcium channel blocker are provided in one formulation. In some embodiments, the diuretic and the calcium channel blocker are provided in one formulation. In some embodiments, the angiotensin II receptor blocker, the diuretic and the calcium channel blocker are provided in one formulation. In some embodiments, the pharmaceutical composition is in the form of a pill, tablet or capsule. In some embodiments, the pharmaceutical composition is in the form of a pill. In some embodiments, the pharmaceutical composition is in the form of a tablet. In some embodiments, the pharmaceutical composition is in the form of a capsule. In some embodiments, the pharmaceutical composition is suitable for oral administration.

Other suitable formulations include but are not limited to those suitable for rectal, topical, buccal, parenteral (e.g., subcutaneous, intramuscular, intradermal, or intravenous) vaginal, or aerosol administration, although the most suitable form of administration in any given case will depend on the degree and severity of the condition being treated and on the nature of the particular compound being used. For example, disclosed compositions may be formulated as a unit dose.

Exemplary pharmaceutical compositions may be used in the form of a pharmaceutical preparation, for example, in solid, semisolid or liquid form, which includes one or more of a disclosed compound as an active ingredient, in admixture with an organic or inorganic carrier or excipient suitable for external, enteral, or parenteral applications. The active ingredient may be compounded, for example, with the usual non-toxic, pharmaceutically acceptable carriers for tablets, pellets, capsules, suppositories, solutions, emulsions, suspensions, and any other form suitable for use. The active object compound is included in the pharmaceutical composition in an amount sufficient to produce the desired effect upon the process or condition of the disease.

For preparing solid compositions such as tablets, the principal active ingredient may be mixed with a pharmaceutical carrier, e.g., conventional tableting ingredients such as corn starch, lactose, sucrose, sorbitol, talc, stearic acid, magnesium stearate, dicalcium phosphate, gums, and other pharmaceutical diluents, e.g., water, to form a solid preformulation composition containing a homogeneous mixture of a disclosed compound or a non-toxic pharmaceutically acceptable salt thereof. When referring to these preformulation compositions as homogeneous, it is meant that the active ingredient is dispersed evenly throughout the composition so that the composition may be readily subdivided into equally effective unit dosage forms such as tablets, pills and capsules.

In solid dosage forms for oral administration (capsules, tablets, pills, dragees, powders, granules and the like), the subject composition is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose, and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such as talc, calcium stearate, magnesium stearate, solid polyethylene glycols, sodium lauryl sulfate, and mixtures thereof; and (10) coloring agents. In the case of capsules, tablets and pills, the compositions may also comprise buffering agents. Solid compositions of a similar type may also be employed as fillers in soft and hard-filled gelatin capsules using excipients such as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the subject composition moistened with an inert liquid diluent. In some embodiments, capsules are prepared by encapsulating tablets in hard-gelatin capsules (e.g., overencapsulation.) Tablets and other solid dosage forms such as dragees, capsules, pills, and granules, may optionally be scored or prepared with coatings and shells, such as enteric coatings and other coatings well known in the pharmaceutical-formulating art.

In some embodiments, the angiotensin II receptor blockers of the pharmaceutical compositions described herein can be replaced with angiotensin converting enzyme inhibitors (ACE inhibitors). Examples of suitable angiotensin converting enzyme inhibitors include but are not limited to benazepril, captopril, enalapril, fosinopril, lisinopril, moexipril, perindopril, quinapril, ramipril, trandolapril or the pharmaceutically acceptable salt or hydrate thereof. In some embodiments, the dose of the angiotensin converting enzyme inhibitor is from about 40% to about 80% of the lowest hypertension therapeutic dose. In some embodiments, the dose of the angiotensin converting enzyme inhibitor is from about 40% to about 70% of the lowest hypertension therapeutic dose. In some embodiments, the dose of the angiotensin converting enzyme inhibitor is from about 40% to about 60% of the lowest hypertension therapeutic dose. In some embodiments, the dose of the angiotensin converting enzyme inhibitor is from about 40% to about 50% of the lowest hypertension therapeutic dose. In some embodiments, the dose of the angiotensin converting enzyme inhibitor is from about 45% to about 55% of the lowest hypertension therapeutic dose. In some embodiments, the dose of the angiotensin converting enzyme inhibitor is from about 50% to about 80% of the lowest hypertension therapeutic dose. In some embodiments, the dose of the angiotensin converting enzyme inhibitor is from about 50% to about 70% of the lowest hypertension therapeutic dose. In some embodiments, the dose of the angiotensin converting enzyme inhibitor is from about 50% to about 60% of the lowest hypertension therapeutic dose. In some embodiments, the dose of the angiotensin converting enzyme inhibitor is from about 60% to about 80% of the lowest hypertension therapeutic dose. In some embodiments, the dose of the angiotensin converting enzyme inhibitor is from about 60% to about 70% of the lowest hypertension therapeutic dose. In some embodiments, the dose of the angiotensin converting enzyme inhibitor is from about 70% to about 80% of the lowest hypertension therapeutic dose. In some embodiments, the dose of the angiotensin converting enzyme inhibitor is about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, or about 80% of the lowest hypertension therapeutic dose. In some embodiments, the dose of the angiotensin converting enzyme inhibitor is about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, or about 60% of the lowest hypertension therapeutic dose. In some embodiments, the dose of the angiotensin converting enzyme inhibitor is about 45%, about 46%, about 47%, about 48%, about 49%, about 50%, about 51%, about 52%, about 53%, about 54%, or about 55% of the lowest hypertension therapeutic dose. In some embodiments, the dose of the angiotensin converting enzyme inhibitor is about 50% of the lowest hypertension therapeutic dose. In some embodiments, the dose of the angiotensin converting enzyme inhibitor is about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, or about 80% of the lowest hypertension therapeutic dose. In some embodiments, the dose of the angiotensin converting enzyme inhibitor is about 61%, about 62%, about 63%, about 64%, about 65%, about 66%, about 67%, about 68%, about 69%, about 70%, or about 71% of the lowest hypertension therapeutic dose. In some embodiments, the dose of the angiotensin converting enzyme inhibitor is about 66% of the lowest hypertension therapeutic dose.

### Methods of Treatment

The pharmaceutical compositions described herein may or may not be administered in a subject who is prescribed with certain other drugs. For instance, the subject could be prescribed with an antihypertensive drug or an antidiabetic drug. In some embodiments, the antihypertensive drug is a renin inhibitor. In some embodiments, the renin inhibitor is pepstatin. In some embodiments, the renin inhibitor is a peptide analogue of the prosegment of renin. In some embodiments, the renin inhibitor is a peptide analogue of the amino-terminal part of the substrate angiotensinogen. In some embodiments, the renin inhibitor is CGP2928. In some embodiments, the renin inhibitor is a peptidomimetic inhibitor. In some embodiments, the renin inhibitor is remikiren, enalkiren, or zankiren. In some embodiments, the renin inhibitor is a non-peptide. In some embodiments, a pharmaceutical composition as described herein is not administered to a subject who is prescribed aliskiren. In some embodiments, a pharmaceutical composition as described herein is not administered to a subject who is taking aliskiren.

In some embodiments, the antihypertensive drug is an inhibitor of substrate CYP3A4. In some embodiments, the antihypertensive drug is an inhibitor of P-glycoprotein. In some embodiments, the antihypertensive drug reduces furosemide blood concentration. In some embodiments, the antihypertensive drug binds to the S3^{bp} binding site of renin. In some embodiments, the antihypertensive drug prevents the conversion of angiotensinogen to angiotensin I. In some embodiments, the antihypertensive drug causes relaxation of blood vessels. In some embodiments, the antihypertensive drug is amtumide. In some embodiments, the antihypertensive drug is tektuma. In some embodiments, the antihypertensive drug is tekturna HCT. In some embodiments, the antihypertensive drug is tekamlo. In some embodiments, the antihypertensive drug is valtuma. In some embodiments, the antihypertensive drug is aliskiren hemifumarate. In some embodiments, the antihypertensive drug is administered orally. In some embodiments, the dosage of the antihypertensive drug is from about 50 mg per day to about 300 mg per day. In some embodiments, the dosage of the antihypertensive drug is about 150 mg per day. In some embodiments, the dosage of the antihypertensive drug is lower than 300 mg per day. In some embodiments, the antihypertensive drug is (2S,4S,5S,7S)-5-amino-N-(2-carbamoyl-2-methylpropyl)-4-hydroxy-2-isopropyl-7-[4-methoxy-3-(3-methoxypropoxy)benzyl]-8-methylnonanamide. In some embodiments, the antihypertensive drug is aliskiren. In some embodiments, the formula of the aliskiren is C₃₀H₅₃N₃O₆. In some embodiments, the structure of the aliskiren is:

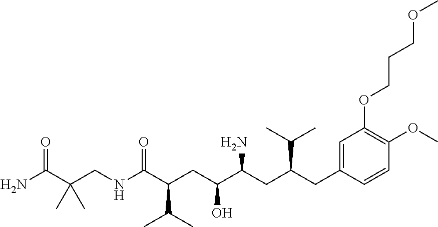 [SMILES: COCCCOc1cc(C[C@@H](C[C@H](N)[C@@H](O)C[C@H](C(=O)NCC(C)(C)C(N)=O)C(C)C)C(C)C)ccc1OC]

The pharmaceutical compositions described herein are useful for treating hypertension in a subject in need thereof. In some embodiments, the subject is administered with a first therapy comprising angiotensin II receptor blocker, diuretic, or calcium channel blocker described herein. In some embodiments, subsequent to the first therapy, the subject is administered with a second therapy comprising angiotensin II receptor blocker, diuretic, or calcium channel blocker described herein. In some embodiments, subsequent to the first therapy and the second therapy, the subject is administered with a third therapy comprising angiotensin II receptor blocker, diuretic, or calcium channel blocker described herein.

In some embodiments, the first therapy comprises: (a) telmisartan as an angiotensin II receptor blocker; (b) indapamide as a thiazide-like diuretic; and (c) amlodipine besylate as a calcium channel blocker. In some embodiments, the first therapy comprises telmisartan at a dose of from about 8 mg to about 12 mg, indapamide at a dose of from about 0.5 mg to about 0.75 mg, and amlodipine besylate at a dose of from about EQ 1 mg base to about EQ 1.5 mg base. In some embodiments, the first therapy comprises telmisartan at a dose of about 10 mg, indapamide at a dose of about 0.625 mg, and amlodipine besylate at a dose of about EQ 1.25 mg base (equivalent to about 1.73 mg amlodipine besylate). In some embodiments, the first therapy comprises telmisartan at a dose of from about 12 mg to about 16 mg, indapamide at a dose of from about 0.75 mg to about 1.0 mg, and amlodipine besylate at a dose of from about EQ 1.5 mg base to about EQ 2 mg base. In some embodiments, the first therapy comprises telmisartan at a dose of about 13.2 mg, indapamide at a dose of about 0.825 mg, and amlodipine besylate at a dose of about EQ 1.65 mg base. In some embodiments, the first therapy comprises telmisartan at a dose of from about 16 mg to about 30 mg, indapamide at a dose of from about 1 mg to about 1.875 mg, and amlodipine besylate at a dose of from about EQ 2 mg base to about EQ 3.75 mg base. In some embodiments, the first therapy comprises telmisartan at a dose of about 20 mg, indapamide at a dose of about 1.25 mg, and amlodipine besylate at a dose of about EQ 2.5 mg base (equivalent to about 3.47 mg amlodipine besylate). In some embodiments, the first therapy comprises telmisartan at a dose of from about 30 mg to about 50 mg, indapamide at a dose of from about 1.875 mg to about 3.125 mg, and amlodipine besylate at a dose of from about EQ 3.75 mg base to about EQ 6.25 mg base. In some embodiments, the first therapy comprises telmisartan at a dose of about 40 mg, indapamide at a dose of about 2.5 mg, and amlodipine besylate at a dose of about EQ 5 mg base (equivalent to about 6.94 mg amlodipine besylate).

In some embodiments, the first therapy comprises: (a) irbesartan as an angiotensin II receptor blocker; (b) hydrochlorothiazide as a thiazide diuretic; and (c) amlodipine besylate as a calcium channel blocker. In some embodiments, the first therapy comprises irbesartan at a dose of from about 30 mg to about 45 mg, hydrochlorothiazide at a dose of from about 5 mg to about 7.5 mg, and amlodipine besylate at a dose of from about EQ 1 mg base to about EQ 1.5 mg base. In some embodiments, the first therapy comprises irbesartan at a dose of about 37.5 mg, hydrochlorothiazide at a dose of about 6.25 mg, and amlodipine besylate at a dose of about EQ 1.25 mg base (equivalent to about 1.73 mg amlodipine besylate). In some embodiments, the first therapy comprises irbesartan at a dose of from about 45 mg to about 60 mg, hydrochlorothiazide at a dose of from about 7.5 mg to about 10 mg, and amlodipine besylate at a dose of from about EQ 1.5 mg base to about EQ 2 mg base. In some embodiments, the first therapy comprises irbesartan at a dose of about 49.5 mg, hydrochlorothiazide at a dose of about 8.25 mg, and amlodipine besylate at a dose of about EQ 1.65 mg base. In some embodiments, the first therapy comprises irbesartan at a dose of from about 60 mg to about 112.5 mg, hydrochlorothiazide at a dose of from about 10 mg to about 18.75 mg, and amlodipine besylate at a dose of from about EQ 2 mg base to about EQ 3.75 mg base. In some embodiments, the first therapy comprises irbesartan at a dose of about 75 mg, hydrochlorothiazide at a dose of about 12.5 mg, and amlodipine besylate at a dose of about EQ 2.5 mg base (equivalent to about 3.47 mg amlodipine besylate). In some embodiments, the first therapy comprises irbesartan at a dose of from about 112.5 mg to about 187.5 mg, hydrochlorothiazide at a dose of from about 18.75 mg to about 31.25 mg, and amlodipine besylate at a dose of from about EQ 3.75 mg base to about EQ 6.25 mg base. In some embodiments, the first therapy comprises irbesartan at a dose of about 150 mg, hydrochlorothiazide at a dose of about 25 mg, and amlodipine besylate at a dose of about EQ 5 mg base (equivalent to about 6.94 mg amlodipine besylate).

In some embodiments, the first therapy comprises: (a) telmisartan as an angiotensin II receptor blocker; (b) hydrochlorothiazide as a thiazide diuretic; and (c) amlodipine besylate as a calcium channel blocker. In some embodiments, the first therapy comprises telmisartan at a dose of from about 8 mg to about 12 mg, hydrochlorothiazide at a dose of from about 5 mg to about 7.5 mg, and amlodipine besylate at a dose of from about EQ 1 mg base to about EQ 1.5 mg base. In some embodiments, the first therapy comprises telmisartan at a dose of about 10 mg, hydrochlorothiazide at a dose of about 6.25 mg, and amlodipine besylate at a dose of about EQ 1.25 mg base (equivalent to about 1.73 mg amlodipine besylate). In some embodiments, the first therapy comprises telmisartan at a dose of from about 12 mg to about 16 mg, hydrochlorothiazide at a dose of from about 7.5 mg to about 10 mg, and amlodipine besylate at a dose of from about EQ 1.5 mg base to about EQ 2 mg base. In some embodiments, the first therapy comprises telmisartan at a dose of about 13.2 mg, hydrochlorothiazide at a dose of about 8.25 mg, and amlodipine besylate at a dose of about EQ 1.65 mg base. In some embodiments, the first therapy comprises telmisartan at a dose of from about 16 mg to about 30 mg, hydrochlorothiazide at a dose of from about 10 mg to about 18.75 mg, and amlodipine besylate at a dose of from about EQ 2 mg base to about EQ 3.75 mg base. In some embodiments, the first therapy comprises telmisartan at a dose of about 20 mg, hydrochlorothiazide at a dose of about 12.5 mg, and amlodipine besylate at a dose of about EQ 2.5 mg base (equivalent to about 3.47 mg amlodipine besylate). In some embodiments, the first therapy comprises telmisartan at a dose of from about 30 mg to about 50 mg, hydrochlorothiazide at a dose of from about 18.75 mg to about 31.25 mg, and amlodipine besylate at a dose of from about EQ 3.75 mg base to about EQ 6.25 mg base. In some embodiments, the first therapy comprises telmisartan at a dose of about 40 mg, hydrochlorothiazide at a dose of about 25 mg, and amlodipine besylate at a dose of about EQ 5 mg base (equivalent to about 6.94 mg amlodipine besylate).

In some embodiments, the first therapy comprises: (a) irbesartan as an angiotensin II receptor blocker; (b) indapamide as a thiazide-like diuretic; and (c) amlodipine besylate as a calcium channel blocker. In some embodiments, the first therapy comprises irbesartan at a dose of from about 30 mg to about 45 mg, indapamide at a dose of from about 0.5 mg to about 0.75 mg, and amlodipine besylate at a dose of from about EQ 1 mg base to about EQ 1.5 mg base. In some embodiments, the first therapy comprises irbesartan at a dose of about 37.5 mg, indapamide at a dose of about 0.625 mg, and amlodipine besylate at a dose of about EQ 1.25 mg base (equivalent to about 1.73 mg amlodipine besylate). In some embodiments, the first therapy comprises irbesartan at a dose of from about 45 mg to about 60 mg, indapamide at a dose of from about 0.75 mg to about 1.0 mg, and amlodipine besylate at a dose of from about EQ 1.5 mg base to about EQ 2 mg base. In some embodiments, the first therapy comprises irbesartan at a dose of about 49.5 mg, indapamide at a dose of about 0.825 mg, and amlodipine besylate at a dose of about EQ 1.65 mg base. In some embodiments, the first therapy comprises irbesartan at a dose of from about 60 mg to about 112.5 mg, indapamide at a dose of from about 1 mg to about 1.875 mg, and amlodipine besylate at a dose of from about EQ 2 mg base to about EQ 3.75 mg base. In some embodiments, the first therapy comprises irbesartan at a dose of about 75 mg, indapamide at a dose of about 1.25 mg, and amlodipine besylate at a dose of about EQ 2.5 mg base (equivalent to about 3.47 mg amlodipine besylate). In some embodiments, the first therapy comprises irbesartan at a dose of from about 112.5 mg to about 187.5 mg, indapamide at a dose of from about 1.875 mg to about 3.125 mg, and amlodipine besylate at a dose of from about EQ 3.75 mg base to about EQ 6.25 mg base. In some embodiments, the first therapy comprises irbesartan at a dose of about 150 mg, indapamide at a dose of about 2.5 mg, and amlodipine besylate at a dose of about EQ 5 mg base (equivalent to about 6.94 mg amlodipine besylate).

In some embodiments, the first therapy comprises: (a) telmisartan as an angiotensin II receptor blocker; (b) chlorthalidone as a thiazide-like diuretic; and (c) amlodipine besylate as a calcium channel blocker. In some embodiments, the first therapy comprises telmisartan at a dose of from about 8 mg to about 12 mg, chlorthalidone at a dose of from about 10 mg to about 15 mg, and amlodipine besylate at a dose of from about EQ 1 mg base to about EQ 1.5 mg base. In some embodiments, the first therapy comprises telmisartan at a dose of about 10 mg, chlorthalidone at a dose of about 12.5 mg, and amlodipine besylate at a dose of about EQ 1.25 mg base (equivalent to about 1.73 mg amlodipine besylate). In some embodiments, the first therapy comprises telmisartan at a dose of from about 12 mg to about 16 mg, chlorthalidone at a dose of from about 15 mg to about 20 mg, and amlodipine besylate at a dose of from about EQ 1.5 mg base to about EQ 2 mg base. In some embodiments, the first therapy comprises telmisartan at a dose of about 13.2 mg, chlorthalidone at a dose of about 16.5 mg, and amlodipine besylate at a dose of about EQ 1.65 mg base. In some embodiments, the first therapy comprises telmisartan at a dose of from about 16 mg to about 30 mg, chlorthalidone at a dose of from about 30 mg to about 37.5 mg, and amlodipine besylate at a dose of from about EQ 2 mg base to about EQ 3.75 mg base. In some embodiments, the first therapy comprises telmisartan at a dose of about 20 mg, chlorthalidone at a dose of about 25 mg, and amlodipine besylate at a dose of about EQ 2.5 mg base (equivalent to about 3.47 mg amlodipine besylate). In some embodiments, the first therapy comprises telmisartan at a dose of from about 30 mg to about 50 mg, chlorthalidone at a dose of from about 37.5 mg to about 62.5 mg, and amlodipine besylate at a dose of from about EQ 3.75 mg base to about EQ 6.25 mg base. In some embodiments, the first therapy comprises telmisartan at a dose of about 40 mg, chlorthalidone at a dose of about 50 mg, and amlodipine besylate at a dose of about EQ 5 mg base (equivalent to about 6.94 mg amlodipine besylate).

In some embodiments, the first therapy comprises: (a) irbesartan as an angiotensin II receptor blocker; (b) chlorthalidone as a thiazide-like diuretic; and (c) amlodipine besylate as a calcium channel blocker. In some embodiments, the first therapy comprises irbesartan at a dose of from about 30 mg to about 45 mg, chlorthalidone at a dose of from about 10 mg to about 15 mg, and amlodipine besylate at a dose of from about EQ 1 mg base to about EQ 1.5 mg base. In some embodiments, the first therapy comprises irbesartan at a dose of about 37.5 mg, chlorthalidone at a dose of about 12.5 mg, and amlodipine besylate at a dose of about EQ 1.25 mg base (equivalent to about 1.73 mg amlodipine besylate). In some embodiments, the first therapy comprises irbesartan at a dose of from about 45 mg to about 60 mg, chlorthalidone at a dose of from about 15 mg to about 20 mg, and amlodipine besylate at a dose of from about EQ 1.5 mg base to about EQ 2 mg base. In some embodiments, the first therapy comprises irbesartan at a dose of about 49.5 mg, chlorthalidone at a dose of about 16.5 mg, and amlodipine besylate at a dose of about EQ 1.65 mg base. In some embodiments, the first therapy comprises irbesartan at a dose of from about 60 mg to about 112.5 mg, chlorthalidone at a dose of from about 20 mg to about 37.5 mg, and amlodipine besylate at a dose of from about EQ 2 mg base to about EQ 3.75 mg base. In some embodiments, the first therapy comprises irbesartan at a dose of about 75 mg, chlorthalidone at a dose of about 25 mg, and amlodipine besylate at a dose of about EQ 2.5 mg base (equivalent to about 3.47 mg amlodipine besylate). In some embodiments, the first therapy comprises irbesartan at a dose of from about 112.5 mg to about 187.5 mg, chlorthalidone at a dose of from about 37.5 mg to about 62.5 mg, and amlodipine besylate at a dose of from about EQ 3.75 mg base to about EQ 6.25 mg base. In some embodiments, the first therapy comprises irbesartan at a dose of about 150 mg, chlorthalidone at a dose of about 50 mg, and amlodipine besylate at a dose of about EQ 5 mg base (equivalent to about 6.94 mg amlodipine besylate).

In some embodiments, the second therapy comprises: (a) telmisartan as an angiotensin II receptor blocker; (b) indapamide as a thiazide-like diuretic; and (c) amlodipine besylate as a calcium channel blocker. In some embodiments, the second therapy comprises telmisartan at a dose of from about 8 mg to about 12 mg, indapamide at a dose of from about 0.5 mg to about 0.75 mg, and amlodipine besylate at a dose of from about EQ 1 mg base to about EQ 1.5 mg base. In some embodiments, the second therapy comprises telmisartan at a dose of about 10 mg, indapamide at a dose of about 0.625 mg, and amlodipine besylate at a dose of about EQ 1.25 mg base (equivalent to about 1.73 mg amlodipine besylate). In some embodiments, the second therapy comprises telmisartan at a dose of from about 12 mg to about 16 mg, indapamide at a dose of from about 0.75 mg to about 1.0 mg, and amlodipine besylate at a dose of from about EQ 1.5 mg base to about EQ 2 mg base. In some embodiments, the second therapy comprises telmisartan at a dose of about 13.2 mg, indapamide at a dose of about 0.825 mg, and amlodipine besylate at a dose of about EQ 1.65 mg base. In some embodiments, the second therapy comprises telmisartan at a dose of from about 16 mg to about 30 mg, indapamide at a dose of from about 1 mg to about 1.875 mg, and amlodipine besylate at a dose of from about EQ 2 mg base to about EQ 3.75 mg base. In some embodiments, the second therapy comprises telmisartan at a dose of about 20 mg, indapamide at a dose of about 1.25 mg, and amlodipine besylate at a dose of about EQ 2.5 mg base (equivalent to about 3.47 mg amlodipine besylate). In some embodiments, the second therapy comprises telmisartan at a dose of from about 30 mg to about 50 mg, indapamide at a dose of from about 1.875 mg to about 3.125 mg, and amlodipine besylate at a dose of from about EQ 3.75 mg base to about EQ 6.25 mg base. In some embodiments, the second therapy comprises telmisartan at a dose of about 40 mg, indapamide at a dose of about 2.5 mg, and amlodipine besylate at a dose of about EQ 5 mg base (equivalent to about 6.94 mg amlodipine besylate).

In some embodiments, the first therapy comprises: (a) irbesartan as an angiotensin II receptor blocker; (b) hydrochlorothiazide as a thiazide diuretic; and (c) amlodipine besylate as a calcium channel blocker. In some embodiments, the first therapy comprises irbesartan at a dose of from about 30 mg to about 45 mg, hydrochlorothiazide at a dose of from about 5 mg to about 7.5 mg, and amlodipine besylate at a dose of from about EQ 1 mg base to about EQ 1.5 mg base. In some embodiments, the first therapy comprises irbesartan at a dose of about 37.5 mg, hydrochlorothiazide at a dose of about 6.25 mg, and amlodipine besylate at a dose of about EQ 1.25 mg base (equivalent to about 1.73 mg amlodipine besylate). In some embodiments, the first therapy comprises irbesartan at a dose of from about 45 mg to about 60 mg, hydrochlorothiazide at a dose of from about 7.5 mg to about 10 mg, and amlodipine besylate at a dose of from about EQ 1.5 mg base to about EQ 2 mg base. In some embodiments, the first therapy comprises irbesartan at a dose of about 49.5 mg, hydrochlorothiazide at a dose of about 8.25 mg, and amlodipine besylate at a dose of about EQ 1.65 mg base. In some embodiments, the first therapy comprises irbesartan at a dose of from about 60 mg to about 112.5 mg, hydrochlorothiazide at a dose of from about 10 mg to about 18.75 mg, and amlodipine besylate at a dose of from about EQ 2 mg base to about EQ 3.75 mg base. In some embodiments, the first therapy comprises irbesartan at a dose of about 75 mg, hydrochlorothiazide at a dose of about 12.5 mg, and amlodipine besylate at a dose of about EQ 2.5 mg base (equivalent to about 3.47 mg amlodipine besylate). In some embodiments, the first therapy comprises irbesartan at a dose of from about 112.5 mg to about 187.5 mg, hydrochlorothiazide at a dose of from about 18.75 mg to about 31.25 mg, and amlodipine besylate at a dose of from about EQ 3.75 mg base to about EQ 6.25 mg base. In some embodiments, the first therapy comprises irbesartan at a dose of about 150 mg, hydrochlorothiazide at a dose of about 25 mg, and amlodipine besylate at a dose of about EQ 5 mg base (equivalent to about 6.94 mg amlodipine besylate).

In some embodiments, the second therapy comprises: (a) irbesartan as an angiotensin II receptor blocker; (b) hydrochlorothiazide as a thiazide diuretic; and (c) amlodipine besylate as a calcium channel blocker. In some embodiments, the second therapy comprises irbesartan at a dose of from about 30 mg to about 45 mg, hydrochlorothiazide at a dose of from about 5 mg to about 7.5 mg, and amlodipine besylate at a dose of from about EQ 1 mg base to about EQ 1.5 mg base. In some embodiments, the second therapy comprises irbesartan at a dose of about 37.5 mg, hydrochlorothiazide at a dose of about 6.25 mg, and amlodipine besylate at a dose of about EQ 1.25 mg base (equivalent to about 1.73 mg amlodipine besylate). In some embodiments, the second therapy comprises irbesartan at a dose of from about 45 mg to about 60 mg, hydrochlorothiazide at a dose of from about 7.5 mg to about 10 mg, and amlodipine besylate at a dose of from about EQ 1.5 mg base to about EQ 2 mg base. In some embodiments, the second therapy comprises irbesartan at a dose of about 49.5 mg, hydrochlorothiazide at a dose of about 8.25 mg, and amlodipine besylate at a dose of about EQ 1.65 mg base. In some embodiments, the second therapy comprises irbesartan at a dose of from about 60 mg to about 112.5 mg, hydrochlorothiazide at a dose of from about 10 mg to about 18.75 mg, and amlodipine besylate at a dose of from about EQ 2 mg base to about EQ 3.75 mg base. In some embodiments, the second therapy comprises irbesartan at a dose of about 75 mg, hydrochlorothiazide at a dose of about 12.5 mg, and amlodipine besylate at a dose of about EQ 2.5 mg base (equivalent to about 3.47 mg amlodipine besylate). In some embodiments, the second therapy comprises irbesartan at a dose of from about 112.5 mg to about 187.5 mg, hydrochlorothiazide at a dose of from about 18.75 mg to about 31.25 mg, and amlodipine besylate at a dose of from about EQ 3.75 mg base to about EQ 6.25 mg base. In some embodiments, the second therapy comprises irbesartan at a dose of about 150 mg, hydrochlorothiazide at a dose of about 25 mg, and amlodipine besylate at a dose of about EQ 5 mg base (equivalent to about 6.94 mg amlodipine besylate).

In some embodiments, the second therapy comprises: (a) telmisartan as an angiotensin II receptor blocker; (b) hydrochlorothiazide as a thiazide diuretic; and (c) amlodipine besylate as a calcium channel blocker. In some embodiments, the second therapy comprises telmisartan at a dose of from about 8 mg to about 12 mg, hydrochlorothiazide at a dose of from about 5 mg to about 7.5 mg, and amlodipine besylate at a dose of from about EQ 1 mg base to about EQ 1.5 mg base. In some embodiments, the second therapy comprises telmisartan at a dose of about 10 mg, hydrochlorothiazide at a dose of about 6.25 mg, and amlodipine besylate at a dose of about EQ 1.25 mg base (equivalent to about 1.73 mg amlodipine besylate). In some embodiments, the second therapy comprises telmisartan at a dose of from about 12 mg to about 16 mg, hydrochlorothiazide at a dose of from about 7.5 mg to about 10 mg, and amlodipine besylate at a dose of from about EQ 1.5 mg base to about EQ 2 mg base. In some embodiments, the second therapy comprises telmisartan at a dose of about 13.2 mg, hydrochlorothiazide at a dose of about 8.25 mg, and amlodipine besylate at a dose of about EQ 1.65 mg base. In some embodiments, the second therapy comprises telmisartan at a dose of from about 16 mg to about 30 mg, hydrochlorothiazide at a dose of from about 10 mg to about 18.75 mg, and amlodipine besylate at a dose of from about EQ 2 mg base to about EQ 3.75 mg base. In some embodiments, the second therapy comprises telmisartan at a dose of about 20 mg, hydrochlorothiazide at a dose of about 12.5 mg, and amlodipine besylate at a dose of about EQ 2.5 mg base (equivalent to about 3.47 mg amlodipine besylate). In some embodiments, the second therapy comprises telmisartan at a dose of from about 30 mg to about 50 mg, hydrochlorothiazide at a dose of from about 18.75 mg to about 31.25 mg, and amlodipine besylate at a dose of from about EQ 3.75 mg base to about EQ 6.25 mg base. In some embodiments, the second therapy comprises telmisartan at a dose of about 40 mg, hydrochlorothiazide at a dose of about 25 mg, and amlodipine besylate at a dose of about EQ 5 mg base (equivalent to about 6.94 mg amlodipine besylate).

In some embodiments, the second therapy comprises: (a) irbesartan as an angiotensin II receptor blocker; (b) indapamide as a thiazide-like diuretic; and (c) amlodipine besylate as a calcium channel blocker. In some embodiments, the second therapy comprises irbesartan at a dose of from about 30 mg to about 45 mg, indapamide at a dose of from about 0.5 mg to about 0.75 mg, and amlodipine besylate at a dose of from about EQ 1 mg base to about EQ 1.5 mg base. In some embodiments, the second therapy comprises irbesartan at a dose of about 37.5 mg, indapamide at a dose of about 0.625 mg, and amlodipine besylate at a dose of about EQ 1.25 mg base (equivalent to about 1.73 mg amlodipine besylate). In some embodiments, the second therapy comprises irbesartan at a dose of from about 45 mg to about 60 mg, indapamide at a dose of from about 0.75 mg to about 1.0 mg, and amlodipine besylate at a dose of from about EQ 1.5 mg base to about EQ 2 mg base. In some embodiments, the second therapy comprises irbesartan at a dose of about 49.5 mg, indapamide at a dose of about 0.825 mg, and amlodipine besylate at a dose of about EQ 1.65 mg base. In some embodiments, the second therapy comprises irbesartan at a dose of from about 60 mg to about 112.5 mg, indapamide at a dose of from about 1 mg to about 1.875 mg, and amlodipine besylate at a dose of from about EQ 2 mg base to about EQ 3.75 mg base. In some embodiments, the second therapy comprises irbesartan at a dose of about 75 mg, indapamide at a dose of about 1.25 mg, and amlodipine besylate at a dose of about EQ 2.5 mg base (equivalent to about 3.47 mg amlodipine besylate). In some embodiments, the second therapy comprises irbesartan at a dose of from about 112.5 mg to about 187.5 mg, indapamide at a dose of from about 1.875 mg to about 3.125 mg, and amlodipine besylate at a dose of from about EQ 3.75 mg base to about EQ 6.25 mg base. In some embodiments, the second therapy comprises irbesartan at a dose of about 150 mg, indapamide at a dose of about 2.5 mg, and amlodipine besylate at a dose of about EQ 5 mg base (equivalent to about 6.94 mg amlodipine besylate).

In some embodiments, the second therapy comprises: (a) telmisartan as an angiotensin II receptor blocker; (b) chlorthalidone as a thiazide-like diuretic; and (c) amlodipine besylate as a calcium channel blocker. In some embodiments, the second therapy comprises telmisartan at a dose of from about 8 mg to about 12 mg, chlorthalidone at a dose of from about 10 mg to about 15 mg, and amlodipine besylate at a dose of from about EQ 1 mg base to about EQ 1.5 mg base. In some embodiments, the second therapy comprises telmisartan at a dose of about 10 mg, chlorthalidone at a dose of about 12.5 mg, and amlodipine besylate at a dose of about EQ 1.25 mg base (equivalent to about 1.73 mg amlodipine besylate). In some embodiments, the second therapy comprises telmisartan at a dose of from about 12 mg to about 16 mg, chlorthalidone at a dose of from about 15 mg to about 20 mg, and amlodipine besylate at a dose of from about EQ 1.5 mg base to about EQ 2 mg base. In some embodiments, the second therapy comprises telmisartan at a dose of about 13.2 mg, chlorthalidone at a dose of about 16.5 mg, and amlodipine besylate at a dose of about EQ 1.65 mg base. In some embodiments, the second therapy comprises telmisartan at a dose of from about 16 mg to about 30 mg, chlorthalidone at a dose of from about 30 mg to about 37.5 mg, and amlodipine besylate at a dose of from about EQ 2 mg base to about EQ 3.75 mg base. In some embodiments, the second therapy comprises telmisartan at a dose of about 20 mg, chlorthalidone at a dose of about 25 mg, and amlodipine besylate at a dose of about EQ 2.5 mg base (equivalent to about 3.47 mg amlodipine besylate). In some embodiments, the second therapy comprises telmisartan at a dose of from about 30 mg to about 50 mg, chlorthalidone at a dose of from about 37.5 mg to about 62.5 mg, and amlodipine besylate at a dose of from about EQ 3.75 mg base to about EQ 6.25 mg base. In some embodiments, the second therapy comprises telmisartan at a dose of about 40 mg, chlorthalidone at a dose of about 50 mg, and amlodipine besylate at a dose of about EQ 5 mg base (equivalent to about 6.94 mg amlodipine besylate).

In some embodiments, the second therapy comprises: (a) irbesartan as an angiotensin II receptor blocker; (b) chlorthalidone as a thiazide-like diuretic; and (c) amlodipine besylate as a calcium channel blocker. In some embodiments, the second therapy comprises irbesartan at a dose of from about 30 mg to about 45 mg, chlorthalidone at a dose of from about 10 mg to about 15 mg, and amlodipine besylate at a dose of from about EQ 1 mg base to about EQ 1.5 mg base. In some embodiments, the second therapy comprises irbesartan at a dose of about 37.5 mg, chlorthalidone at a dose of about 12.5 mg, and amlodipine besylate at a dose of about EQ 1.25 mg base (equivalent to about 1.73 mg amlodipine besylate). In some embodiments, the second therapy comprises irbesartan at a dose of from about 45 mg to about 60 mg, chlorthalidone at a dose of from about 15 mg to about 20 mg, and amlodipine besylate at a dose of from about EQ 1.5 mg base to about EQ 2 mg base. In some embodiments, the second therapy comprises irbesartan at a dose of about 49.5 mg, chlorthalidone at a dose of about 16.5 mg, and amlodipine besylate at a dose of about EQ 1.65 mg base. In some embodiments, the second therapy comprises irbesartan at a dose of from about 60 mg to about 112.5 mg, chlorthalidone at a dose of from about 20 mg to about 37.5 mg, and amlodipine besylate at a dose of from about EQ 2 mg base to about EQ 3.75 mg base. In some embodiments, the second therapy comprises irbesartan at a dose of about 75 mg, chlorthalidone at a dose of about 25 mg, and amlodipine besylate at a dose of about EQ 2.5 mg base (equivalent to about 3.47 mg amlodipine besylate). In some embodiments, the second therapy comprises irbesartan at a dose of from about 112.5 mg to about 187.5 mg, chlorthalidone at a dose of from about 37.5 mg to about 62.5 mg, and amlodipine besylate at a dose of from about EQ 3.75 mg base to about EQ 6.25 mg base. In some embodiments, the second therapy comprises irbesartan at a dose of about 150 mg, chlorthalidone at a dose of about 50 mg, and amlodipine besylate at a dose of about EQ 5 mg base (equivalent to about 6.94 mg amlodipine besylate).

In some embodiments, the second therapy comprises: (a) irbesartan as an angiotensin II receptor blocker; (b) hydrochlorothiazide as a thiazide diuretic; and (c) amlodipine besylate as a calcium channel blocker. In some embodiments, the second therapy comprises irbesartan at a dose of from about 30 mg to about 45 mg, hydrochlorothiazide at a dose of from about 5 mg to about 7.5 mg, and amlodipine besylate at a dose of from about EQ 1 mg base to about EQ 1.5 mg base. In some embodiments, the second therapy comprises irbesartan at a dose of about 37.5 mg, hydrochlorothiazide at a dose of about 6.25 mg, and amlodipine besylate at a dose of about EQ 1.25 mg base (equivalent to about 1.73 mg amlodipine besylate). In some embodiments, the second therapy comprises irbesartan at a dose of from about 45 mg to about 60 mg, hydrochlorothiazide at a dose of from about 7.5 mg to about 10 mg, and amlodipine besylate at a dose of from about EQ 1.5 mg base to about EQ 2 mg base. In some embodiments, the second therapy comprises irbesartan at a dose of about 49.5 mg, hydrochlorothiazide at a dose of about 8.25 mg, and amlodipine besylate at a dose of about EQ 1.65 mg base. In some embodiments, the second therapy comprises irbesartan at a dose of from about 60 mg to about 112.5 mg, hydrochlorothiazide at a dose of from about 10 mg to about 18.75 mg, and amlodipine besylate at a dose of from about EQ 2 mg base to about EQ 3.75 mg base. In some embodiments, the second therapy comprises irbesartan at a dose of about 75 mg, hydrochlorothiazide at a dose of about 12.5 mg, and amlodipine besylate at a dose of about EQ 2.5 mg base (equivalent to about 3.47 mg amlodipine besylate). In some embodiments, the second therapy comprises irbesartan at a dose of from about 112.5 mg to about 187.5 mg, hydrochlorothiazide at a dose of from about 18.75 mg to about 31.25 mg, and amlodipine besylate at a dose of from about EQ 3.75 mg base to about EQ 6.25 mg base. In some embodiments, the second therapy comprises irbesartan at a dose of about 150 mg, hydrochlorothiazide at a dose of about 25 mg, and amlodipine besylate at a dose of about EQ 5 mg base (equivalent to about 6.94 mg amlodipine besylate).

In some embodiments, the third therapy comprises: (a) telmisartan as an angiotensin II receptor blocker; (b) indapamide as a thiazide-like diuretic; and (c) amlodipine besylate as a calcium channel blocker. In some embodiments, the third therapy comprises telmisartan at a dose of from about 8 mg to about 12 mg, indapamide at a dose of from about 0.5 mg to about 0.75 mg, and amlodipine besylate at a dose of from about EQ 1 mg base to about EQ 1.5 mg base. In some embodiments, the third therapy comprises telmisartan at a dose of about 10 mg, indapamide at a dose of about 0.625 mg, and amlodipine besylate at a dose of about EQ 1.25 mg base (equivalent to about 1.73 mg amlodipine besylate). In some embodiments, the third therapy comprises telmisartan at a dose of from about 12 mg to about 16 mg, indapamide at a dose of from about 0.75 mg to about 1.0 mg, and amlodipine besylate at a dose of from about EQ 1.5 mg base to about EQ 2 mg base. In some embodiments, the third therapy comprises telmisartan at a dose of about 13.2 mg, indapamide at a dose of about 0.825 mg, and amlodipine besylate at a dose of about EQ 1.65 mg base. In some embodiments, the third therapy comprises telmisartan at a dose of from about 16 mg to about 30 mg, indapamide at a dose of from about 1 mg to about 1.875 mg, and amlodipine besylate at a dose of from about EQ 2 mg base to about EQ 3.75 mg base. In some embodiments, the third therapy comprises telmisartan at a dose of about 20 mg, indapamide at a dose of about 1.25 mg, and amlodipine besylate at a dose of about EQ 2.5 mg base (equivalent to about 3.47 mg amlodipine besylate). In some embodiments, the third therapy comprises telmisartan at a dose of from about 30 mg to about 50 mg, indapamide at a dose of from about 1.875 mg to about 3.125 mg, and amlodipine besylate at a dose of from about EQ 3.75 mg base to about EQ 6.25 mg base. In some embodiments, the third therapy comprises telmisartan at a dose of about 40 mg, indapamide at a dose of about 2.5 mg, and amlodipine besylate at a dose of about EQ 5 mg base (equivalent to about 6.94 mg amlodipine besylate).

In some embodiments, the third therapy comprises: (a) irbesartan as an angiotensin II receptor blocker; (b) hydrochlorothiazide as a thiazide diuretic; and (c) amlodipine besylate as a calcium channel blocker. In some embodiments, the third therapy comprises irbesartan at a dose of from about 30 mg to about 45 mg, hydrochlorothiazide at a dose of from about 5 mg to about 7.5 mg, and amlodipine besylate at a dose of from about EQ 1 mg base to about EQ 1.5 mg base. In some embodiments, the third therapy comprises irbesartan at a dose of about 37.5 mg, hydrochlorothiazide at a dose of about 6.25 mg, and amlodipine besylate at a dose of about EQ 1.25 mg base (equivalent to about 1.73 mg amlodipine besylate). In some embodiments, the third therapy comprises irbesartan at a dose of from about 45 mg to about 60 mg, hydrochlorothiazide at a dose of from about 7.5 mg to about 10 mg, and amlodipine besylate at a dose of from about EQ 1.5 mg base to about EQ 2 mg base. In some embodiments, the third therapy comprises irbesartan at a dose of about 49.5 mg, hydrochlorothiazide at a dose of about 8.25 mg, and amlodipine besylate at a dose of about EQ 1.65 mg base. In some embodiments, the third therapy comprises irbesartan at a dose of from about 60 mg to about 112.5 mg, hydrochlorothiazide at a dose of from about 10 mg to about 18.75 mg, and amlodipine besylate at a dose of from about EQ 2 mg base to about EQ 3.75 mg base. In some embodiments, the third therapy comprises irbesartan at a dose of about 75 mg, hydrochlorothiazide at a dose of about 12.5 mg, and amlodipine besylate at a dose of about EQ 2.5 mg base (equivalent to about 3.47 mg amlodipine besylate). In some embodiments, the third therapy comprises irbesartan at a dose of from about 112.5 mg to about 187.5 mg, hydrochlorothiazide at a dose of from about 18.75 mg to about 31.25 mg, and amlodipine besylate at a dose of from about EQ 3.75 mg base to about EQ 6.25 mg base. In some embodiments, the third therapy comprises irbesartan at a dose of about 150 mg, hydrochlorothiazide at a dose of about 25 mg, and amlodipine besylate at a dose of about EQ 5 mg base (equivalent to about 6.94 mg amlodipine besylate).

In some embodiments, the third therapy comprises: (a) telmisartan as an angiotensin II receptor blocker; (b) hydrochlorothiazide as a thiazide diuretic; and (c) amlodipine besylate as a calcium channel blocker. In some embodiments, the third therapy comprises telmisartan at a dose of from about 8 mg to about 12 mg, hydrochlorothiazide at a dose of from about 5 mg to about 7.5 mg, and amlodipine besylate at a dose of from about EQ 1 mg base to about EQ 1.5 mg base. In some embodiments, the third therapy comprises telmisartan at a dose of about 10 mg, hydrochlorothiazide at a dose of about 6.25 mg, and amlodipine besylate at a dose of about EQ 1.25 mg base (equivalent to about 1.73 mg amlodipine besylate). In some embodiments, the third therapy comprises telmisartan at a dose of from about 12 mg to about 16 mg, hydrochlorothiazide at a dose of from about 7.5 mg to about 10 mg, and amlodipine besylate at a dose of from about EQ 1.5 mg base to about EQ 2 mg base. In some embodiments, the third therapy comprises telmisartan at a dose of about 13.2 mg, hydrochlorothiazide at a dose of about 8.25 mg, and amlodipine besylate at a dose of about EQ 1.65 mg base. In some embodiments, the third therapy comprises telmisartan at a dose of from about 16 mg to about 30 mg, hydrochlorothiazide at a dose of from about 10 mg to about 18.75 mg, and amlodipine besylate at a dose of from about EQ 2 mg base to about EQ 3.75 mg base. In some embodiments, the third therapy comprises telmisartan at a dose of about 20 mg, hydrochlorothiazide at a dose of about 12.5 mg, and amlodipine besylate at a dose of about EQ 2.5 mg base (equivalent to about 3.47 mg amlodipine besylate). In some embodiments, the third therapy comprises telmisartan at a dose of from about 30 mg to about 50 mg, hydrochlorothiazide at a dose of from about 18.75 mg to about 31.25 mg, and amlodipine besylate at a dose of from about EQ 3.75 mg base to about EQ 6.25 mg base. In some embodiments, the third therapy comprises telmisartan at a dose of about 40 mg, hydrochlorothiazide at a dose of about 25 mg, and amlodipine besylate at a dose of about EQ 5 mg base (equivalent to about 6.94 mg amlodipine besylate).

In some embodiments, the third therapy comprises: (a) irbesartan as an angiotensin II receptor blocker; (b) indapamide as a thiazide-like diuretic; and (c) amlodipine besylate as a calcium channel blocker. In some embodiments, the third therapy comprises irbesartan at a dose of from about 30 mg to about 45 mg, indapamide at a dose of from about 0.5 mg to about 0.75 mg, and amlodipine besylate at a dose of from about EQ 1 mg base to about EQ 1.5 mg base. In some embodiments, the third therapy comprises irbesartan at a dose of about 37.5 mg, indapamide at a dose of about 0.625 mg, and amlodipine besylate at a dose of about EQ 1.25 mg base (equivalent to about 1.73 mg amlodipine besylate). In some embodiments, the third therapy comprises irbesartan at a dose of from about 45 mg to about 60 mg, indapamide at a dose of from about 0.75 mg to about 1.0 mg, and amlodipine besylate at a dose of from about EQ 1.5 mg base to about EQ 2 mg base. In some embodiments, the third therapy comprises irbesartan at a dose of about 49.5 mg, indapamide at a dose of about 0.825 mg, and amlodipine besylate at a dose of about EQ 1.65 mg base. In some embodiments, the third therapy comprises irbesartan at a dose of from about 60 mg to about 112.5 mg, indapamide at a dose of from about 1 mg to about 1.875 mg, and amlodipine besylate at a dose of from about EQ 2 mg base to about EQ 3.75 mg base. In some embodiments, the third therapy comprises irbesartan at a dose of about 75 mg, indapamide at a dose of about 1.25 mg, and amlodipine besylate at a dose of about EQ 2.5 mg base (equivalent to about 3.47 mg amlodipine besylate). In some embodiments, the third therapy comprises irbesartan at a dose of from about 112.5 mg to about 187.5 mg, indapamide at a dose of from about 1.875 mg to about 3.125 mg, and amlodipine besylate at a dose of from about EQ 3.75 mg base to about EQ 6.25 mg base. In some embodiments, the third therapy comprises irbesartan at a dose of about 150 mg, indapamide at a dose of about 2.5 mg, and amlodipine besylate at a dose of about EQ 5 mg base (equivalent to about 6.94 mg amlodipine besylate).

In some embodiments, the third therapy comprises: (a) telmisartan as an angiotensin II receptor blocker; (b) chlorthalidone as a thiazide-like diuretic; and (c) amlodipine besylate as a calcium channel blocker. In some embodiments, the third therapy comprises telmisartan at a dose of from about 8 mg to about 12 mg, chlorthalidone at a dose of from about 10 mg to about 15 mg, and amlodipine besylate at a dose of from about EQ 1 mg base to about EQ 1.5 mg base. In some embodiments, the third therapy comprises telmisartan at a dose of about 10 mg, chlorthalidone at a dose of about 12.5 mg, and amlodipine besylate at a dose of about EQ 1.25 mg base (equivalent to about 1.73 mg amlodipine besylate). In some embodiments, the third therapy comprises telmisartan at a dose of from about 12 mg to about 16 mg, chlorthalidone at a dose of from about 15 mg to about 20 mg, and amlodipine besylate at a dose of from about EQ 1.5 mg base to about EQ 2 mg base. In some embodiments, the third therapy comprises telmisartan at a dose of about 13.2 mg, chlorthalidone at a dose of about 16.5 mg, and amlodipine besylate at a dose of about EQ 1.65 mg base. In some embodiments, the third therapy comprises telmisartan at a dose of from about 16 mg to about 30 mg, chlorthalidone at a dose of from about 30 mg to about 37.5 mg, and amlodipine besylate at a dose of from about EQ 2 mg base to about EQ 3.75 mg base. In some embodiments, the third therapy comprises telmisartan at a dose of about 20 mg, chlorthalidone at a dose of about 25 mg, and amlodipine besylate at a dose of about EQ 2.5 mg base (equivalent to about 3.47 mg amlodipine besylate). In some embodiments, the third therapy comprises telmisartan at a dose of from about 30 mg to about 50 mg, chlorthalidone at a dose of from about 37.5 mg to about 62.5 mg, and amlodipine besylate at a dose of from about EQ 3.75 mg base to about EQ 6.25 mg base. In some embodiments, the third therapy comprises telmisartan at a dose of about 40 mg, chlorthalidone at a dose of about 50 mg, and amlodipine besylate at a dose of about EQ 5 mg base (equivalent to about 6.94 mg amlodipine besylate).

In some embodiments, the third therapy comprises: (a) irbesartan as an angiotensin II receptor blocker; (b) chlorthalidone as a thiazide-like diuretic; and (c) amlodipine besylate as a calcium channel blocker. In some embodiments, the third therapy comprises irbesartan at a dose of from about 30 mg to about 45 mg, chlorthalidone at a dose of from about 10 mg to about 15 mg, and amlodipine besylate at a dose of from about EQ 1 mg base to about EQ 1.5 mg base. In some embodiments, the third therapy comprises irbesartan at a dose of about 37.5 mg, chlorthalidone at a dose of about 12.5 mg, and amlodipine besylate at a dose of about EQ 1.25 mg base (equivalent to about 1.73 mg amlodipine besylate). In some embodiments, the third therapy comprises irbesartan at a dose of from about 45 mg to about 60 mg, chlorthalidone at a dose of from about 15 mg to about 20 mg, and amlodipine besylate at a dose of from about EQ 1.5 mg base to about EQ 2 mg base. In some embodiments, the third therapy comprises irbesartan at a dose of about 49.5 mg, chlorthalidone at a dose of about 16.5 mg, and amlodipine besylate at a dose of about EQ 1.65 mg base. In some embodiments, the third therapy comprises irbesartan at a dose of from about 60 mg to about 112.5 mg, chlorthalidone at a dose of from about 20 mg to about 37.5 mg, and amlodipine besylate at a dose of from about EQ 2 mg base to about EQ 3.75 mg base. In some embodiments, the third therapy comprises irbesartan at a dose of about 75 mg, chlorthalidone at a dose of about 25 mg, and amlodipine besylate at a dose of about EQ 2.5 mg base (equivalent to about 3.47 mg amlodipine besylate). In some embodiments, the third therapy comprises irbesartan at a dose of from about 112.5 mg to about 187.5 mg, chlorthalidone at a dose of from about 37.5 mg to about 62.5 mg, and amlodipine besylate at a dose of from about EQ 3.75 mg base to about EQ 6.25 mg base. In some embodiments, the third therapy comprises irbesartan at a dose of about 150 mg, chlorthalidone at a dose of about 50 mg, and amlodipine besylate at a dose of about EQ 5 mg base (equivalent to about 6.94 mg amlodipine besylate).

In some embodiments, the third therapy comprises: (a) irbesartan as an angiotensin II receptor blocker; (b) hydrochlorothiazide as a thiazide diuretic; and (c) amlodipine besylate as a calcium channel blocker. In some embodiments, the third therapy comprises irbesartan at a dose of from about 30 mg to about 45 mg, hydrochlorothiazide at a dose of from about 5 mg to about 7.5 mg, and amlodipine besylate at a dose of from about EQ 1 mg base to about EQ 1.5 mg base. In some embodiments, the third therapy comprises irbesartan at a dose of about 37.5 mg, hydrochlorothiazide at a dose of about 6.25 mg, and amlodipine besylate at a dose of about EQ 1.25 mg base (equivalent to about 1.73 mg amlodipine besylate). In some embodiments, the third therapy comprises irbesartan at a dose of from about 45 mg to about 60 mg, hydrochlorothiazide at a dose of from about 7.5 mg to about 10 mg, and amlodipine besylate at a dose of from about EQ 1.5 mg base to about EQ 2 mg base. In some embodiments, the third therapy comprises irbesartan at a dose of about 49.5 mg, hydrochlorothiazide at a dose of about 8.25 mg, and amlodipine besylate at a dose of about EQ 1.65 mg base. In some embodiments, the third therapy comprises irbesartan at a dose of from about 60 mg to about 112.5 mg, hydrochlorothiazide at a dose of from about 10 mg to about 18.75 mg, and amlodipine besylate at a dose of from about EQ 2 mg base to about EQ 3.75 mg base. In some embodiments, the third therapy comprises irbesartan at a dose of about 75 mg, hydrochlorothiazide at a dose of about 12.5 mg, and amlodipine besylate at a dose of about EQ 2.5 mg base (equivalent to about 3.47 mg amlodipine besylate). In some embodiments, the third therapy comprises irbesartan at a dose of from about 112.5 mg to about 187.5 mg, hydrochlorothiazide at a dose of from about 18.75 mg to about 31.25 mg, and amlodipine besylate at a dose of from about EQ 3.75 mg base to about EQ 6.25 mg base. In some embodiments, the third therapy comprises irbesartan at a dose of about 150 mg, hydrochlorothiazide at a dose of about 25 mg, and amlodipine besylate at a dose of about EQ 5 mg base (equivalent to about 6.94 mg amlodipine besylate).

In some embodiments, the treatment results in a systolic blood pressure (SBP) of less than about 140 mmHg. In some embodiments, the treatment results in a systolic blood pressure (SBP) of less than about 135 mmHg. In some embodiments, the treatment results in a reduction of systolic blood pressure (SBP) of about 10 mmHg or greater. In some embodiments, the treatment results in a reduction of systolic blood pressure (SBP) of about 10 mmHg to about 20 mmHg. In some embodiments, the treatment results in a reduction of systolic blood pressure (SBP) of about 10 mmHg to about 30 mmHg. In some embodiments, the treatment results in a reduction of systolic blood pressure (SBP) of about 10 mmHg, about 11 mmHg, about 12 mmHg, about 13 mmHg, about 14 mmHg, about 15 mmHg, about 16 mmHg, about 17 mmHg, about 18 mmHg, about 19 mmHg, or about 20 mmHg. In some embodiments, the treatment results in a reduction of systolic blood pressure (SBP) of about 10 mmHg, about 11 mmHg, about 12 mmHg, about 13 mmHg, about 14 mmHg, about 15 mmHg, about 16 mmHg, about 17 mmHg, about 18 mmHg, about 19 mmHg, about 20 mmHg, about 21 mmHg, about 22 mmHg, about 23 mmHg, about 24 mmHg, about 25 mmHg, about 26 mmHg, about 27 mmHg, about 28 mmHg, about 29 mmHg, or about 30 mmHg. In some embodiments, the treatment results in a diastolic blood pressure (DBP) of less than about 90 mmHg. In some embodiments, the treatment results in a diastolic blood pressure (DBP) of less than about 85 mmHg. In some embodiments, treatment results in a reduction of diastolic blood pressure (DBP) of about 5 mmHg or greater. In some embodiments, treatment results in a reduction of diastolic blood pressure (DBP) of about 5 mmHg to about 10 mmHg. In some embodiments, treatment results in a reduction of diastolic blood pressure (DBP) of about 5 mmHg to about 15 mmHg. In some embodiments, treatment results in a reduction of diastolic blood pressure (DBP) of about 5 mmHg, about 6 mmHg, about 7 mmHg, about 8 mmHg, about 9 mmHg, or about 10 mmHg. In some embodiments, treatment results in a reduction of diastolic blood pressure (DBP) of about 5 mmHg, about 6 mmHg, about 7 mmHg, about 8 mmHg, about 9 mmHg, about 10 mmHg, about 11 mmHg, about 12 mmHg, about 13 mmHg, about 14 mmHg, or about 15 mmHg.

In some embodiments, treatment results in a reduction in systolic blood pressure (SBP) that is greater than the reduction obtained with the full lowest hypertension therapeutic dose of any one of the angiotensin II receptor blockers, the diuretic, and the calcium channel blocker in the pharmaceutical composition. In some embodiments, treatment results in a reduction in systolic blood pressure (SBP) that is greater than the reduction obtained with the full lowest hypertension therapeutic dose of the angiotensin II receptor blocker in the pharmaceutical composition. In some embodiments, treatment results in a reduction in systolic blood pressure (SBP) that is greater than the reduction obtained with the full lowest hypertension therapeutic dose of the diuretic in the pharmaceutical composition. In some embodiments, treatment results in a reduction in systolic blood pressure (SBP) that is greater than the reduction obtained with the full lowest hypertension therapeutic dose of the calcium channel blocker in the pharmaceutical composition.

In some embodiments, treatment results in a reduction in diastolic blood pressure (DBP) that is greater than the reduction obtained with the full lowest hypertension therapeutic dose of any one of the angiotensin II receptor blockers, the diuretic, and the calcium channel blocker in the pharmaceutical composition. In some embodiments, treatment results in a reduction in diastolic blood pressure (DBP) that is greater than the reduction obtained with the full lowest hypertension therapeutic dose of the angiotensin II receptor blocker in the pharmaceutical composition. In some embodiments, treatment results in a reduction in diastolic blood pressure (DBP) that is greater than the reduction obtained with the full lowest hypertension therapeutic dose of the diuretic in the pharmaceutical composition. In some embodiments, treatment results in a reduction in diastolic blood pressure (DBP) that is greater than the reduction obtained with the full lowest hypertension therapeutic dose of the calcium channel blocker in the pharmaceutical composition.

In some embodiments, treatment results in greater long-term tolerability and reduced risk of side effects when compared to treatment with the full lowest hypertension therapeutic dose of any one of the angiotensin II receptor blockers, the diuretic, and the calcium channel blocker in the pharmaceutical composition. In some embodiments, the treatment results in greater long-term tolerability and reduced risk of side effects when compared to treatment with the full lowest hypertension therapeutic dose of the angiotensin II receptor blocker in the pharmaceutical composition. In some embodiments, the treatment results in greater long-term tolerability and reduced risk of side effects when compared to treatment with the full lowest hypertension therapeutic dose of the diuretic in the pharmaceutical composition. In some embodiments, the treatment results in greater long-term tolerability and reduced risk of side effects when compared to treatment with the full lowest hypertension therapeutic dose of the calcium channel blocker in the pharmaceutical composition.

In some embodiments, treatment results in a reduction in systolic blood pressure (SBP) that is greater than or equal to the reduction obtained with the combination of any two of the angiotensin II receptor blockers, the diuretic, and the calcium channel blocker in the pharmaceutical composition, wherein the dose of each angiotensin II receptor blocker, the diuretic, and the calcium channel blocker is about 50% of the lowest hypertension therapeutic dose. In some embodiments, treatment results in a reduction in diastolic blood pressure (DBP) that is greater than or equal to the reduction obtained with a combination of any two of the angiotensin II receptor blocker, the diuretic, and the calcium channel blocker in the pharmaceutical composition, wherein the dose of each the angiotensin II receptor blocker, the diuretic, and the calcium channel blocker is about 50% of the lowest hypertension therapeutic dose. In some embodiments, the treatment results in greater long-term tolerability and reduced risk of side effects when compared to treatment with a combination of any two of the angiotensin II receptor blockers, the diuretic, and the calcium channel blocker in the pharmaceutical composition, wherein the dose of each the angiotensin II receptor blocker, the diuretic, and the calcium channel blocker is about 50% of the lowest hypertension therapeutic dose.

In some embodiments, the treatment is the initial or first-line treatment of hypertension. In some embodiments, the subject has a very mild elevation of blood pressure prior to treatment. In some embodiments, the subject is not on any previous hypertension therapy prior to treatment. In some embodiments, the subject has a very mild elevation of blood pressure prior to treatment and is not on any previous hypertension therapy prior to treatment.

### EMBODIMENTS

Embodiment 1. A method of treating a subject having hypertension, the method comprising administering a pharmaceutical composition comprising: (a) telmisartan; (b) indapamide; and (c) amlodipine besylate, wherein said pharmaceutical composition is not administered in a subject who is prescribed aliskiren.

Embodiment 2. The method of embodiment 1, wherein the pharmaceutical composition is formulated for oral administration.

Embodiment 3. The method of embodiment 1 or 2, wherein the pharmaceutical composition is in the form of a pill, tablet, or capsule.

Embodiment 4. The method of any one of the preceding embodiments, wherein the treatment results in a systolic blood pressure (SBP) of less than about 140 mmHg.

Embodiment 5. The method of any one of the preceding embodiments, wherein the treatment results in a reduction of systolic blood pressure (SBP) of about 10 mmHg or greater.

Embodiment 6. The method of any one of the preceding embodiments, wherein the treatment results in a diastolic blood pressure (DBP) of less than about 90 mmHg.

Embodiment 7. The method of any one of the preceding embodiments, wherein the treatment results in a reduction of diastolic blood pressure (DBP) of about 5 mmHg or greater.

Embodiment 8. The method of any one of the preceding embodiments, wherein treatment results in a reduction in systolic blood pressure (SBP) that is greater than the reduction obtained with the full lowest hypertension therapeutic dose of any one of the (a), (b), and (c) in the pharmaceutical composition.

Embodiment 9. The method of any one of the preceding embodiments, wherein treatment results in a reduction in diastolic blood pressure (DBP) that is greater than the reduction obtained with the full lowest hypertension therapeutic dose of any one of (a), (b), and (c) in the pharmaceutical composition.

Embodiment 10. The method of any one of the preceding embodiments, wherein the treatment results in greater long-term tolerability and reduced risk of side effects when compared to treatment with the full lowest hypertension therapeutic dose of any one of (a), (b), and (c) in the pharmaceutical composition.

Embodiment 11. The method of any one of the preceding embodiments, wherein the treatment is an initial or first-line treatment of hypertension.

Embodiment 11. The method of any one of embodiments 1-9, wherein the treatment is not an initial or first-line treatment of hypertension.

Embodiment 12. The method of any one of embodiments 1-9 or 11, wherein: (a) the subject is administered with a first therapy comprising (i) telmisartan at a dose of about 10 mg, (ii) indapamide at a dose of about 0.625 mg, and (iii) amlodipine besylate at a dose of about EQ 1.25 mg base; (b) subsequent to the first therapy of (a), the subject is administered with a second therapy comprising (i) telmisartan at a dose of about 20 mg, (ii) indapamide at a dose of about 1.25 mg, and (iii) amlodipine besylate at a dose of about EQ 2.5 mg base; and (c) subsequent to the first therapy of (a) and the second therapy of (b), the subject is administered with a third therapy comprising (i) telmisartan at a dose of about 40 mg, (ii) indapamide at a dose of about 2.5 mg, and (iii) amlodipine besylate at a dose of about EQ 5 mg base.

Embodiment 13. The method of any one of embodiments 1-11, wherein a dose of indapamide is about 0.625 mg.

Embodiment 14. The method of any one of embodiments 1-11 or 13, wherein a dose of amlodipine besylate is about EQ 1.25 mg base.

Embodiment 15. The method of any one of embodiments 1-11 or 13-14, wherein a dose of telmisartan is about 10 mg.

Embodiment 16. The method of any one of embodiments 1-11 or 13-15, wherein a dose of indapamide is about 0.625 mg, a dose of amlodipine besylate is about EQ 1.25 mg base, and a dose of telmisartan is about 10 mg.

Embodiment 17. The method of any one of embodiments 1-11, wherein a dose of indapamide is about 1.25 mg.

Embodiment 18. The method of any one of embodiments 1-11 or 17, wherein a dose of amlodipine besylate is about EQ 2.5 mg base.

Embodiment 19. The method of any one of embodiments 1-11 or 17-18, wherein a dose of telmisartan is about 20 mg.

Embodiment 20. The method of any one of embodiments 1-11 or 17-19, wherein a dose of indapamide is about 1.25 mg, a dose of amlodipine besylate is about EQ 2.5 mg base, and a dose of telmisartan is about 20 mg.

Embodiment 21. The method of any one of embodiments 1-11, wherein a dose of indapamide is about 2.5 mg.

Embodiment 22. The method of any one of embodiments 1-11 or 21, wherein a dose of amlodipine besylate is about EQ 5 mg base.

Embodiment 23. The method of any one of embodiments 1-11 or 21-22, wherein a dose of telmisartan is about 40 mg.

Embodiment 24. The method of any one of embodiments 1-11 or 21-23, wherein a dose of indapamide is about 2.5 mg, a dose of amlodipine besylate is about EQ 5 mg base, and a dose of telmisartan is about 40 mg.

This present disclosure recognizes that the use of the angiotensin II receptor blocker in the pharmaceutical compositions disclosed herein in some embodiments provides beneficial therapeutic effects, which include but are not limited to significant reduction in blood pressure, significant reduction in blood pressure among subjects with mild elevation in blood pressure, greater long-term tolerability, and reduced risk of side effects. This present disclosure recognizes that the exclusion of a lipid-regulating agent, a platelet function altering agent, a serum homocysteine lowering agent, or a combination thereof in the pharmaceutical compositions disclosed herein in some embodiments provides beneficial therapeutic effects, which include but are not limited to significant reduction in blood pressure, significant reduction in blood pressure among subjects with mild elevation in blood pressure, greater long-term tolerability, and reduced risk of side effects.

### EXAMPLES

### Example 1: Cardiovascular Measurement in Spontaneously Hypertensive Rats Receiving Combinations of Anti-Hypertensive Drugs

### Summary

The purpose of this study was to evaluate the comparative effects on blood pressure of three different combinations of an angiotensin II receptor blocker, a calcium channel blocker, and a diuretic (a thiazide diuretic or a thiazide-like diuretic). The main objectives were to assess whether there were differences in the effects of combinations utilizing different drugs from the same class, and differences between combinations utilizing the same drugs at different doses, including very low doses (i.e., doses below the lowest doses approved and manufactured, such as those at 50% of the lowest hypertension therapeutic dose (LHTD)).

The specific combinations studied were:
Combination 1: telmisartan, amlodipine besylate, and indapamide, all at 50% of the lowest hypertension therapeutic dose (LHTD) or one-quarter of the FDA-recommended usual maintenance dose (corresponding to telmisartan 10 mg, amlodipine besylate EQ 1.25 mg base (equivalent to about 1.73 mg amlodipine besylate), and indapamide 0.625 mg);
Combination 2: telmisartan, amlodipine, and indapamide, all at 100% of the lowest hypertension therapeutic dose (LHTD) or one-half of the FDA-recommended usual maintenance dose (corresponding to telmisartan 20 mg, amlodipine besylate 2.5 mg (equivalent to about 3.47 mg amlodipine besylate), and indapamide 1.25 mg); and
Combination 3: losartan, amlodipine besylate, and hydrochlorothiazide, all at 100% of the lowest hypertension therapeutic dose (LHTD) or one-half of the FDA-recommended usual maintenance dose (corresponding to losartan 25 mg, amlodipine besylate 2.5 mg (equivalent to about 3.47 mg amlodipine besylate), and hydrochlorothiazide 12.5 mg).

The study was conducted in spontaneously hypertensive rats (SHR), the most commonly used animal model for the study of hypertension (see Pinto Y M, Paul M, Ganten D. “Lessons from rat models of hypertension: from Goldblatt to genetic engineering”. Cardiovascular Research. 39 (1): 77-88.). Drug doses were calculated using standard allometric scaling methods and data from the published literature on Cmax and AUC for each of the six antihypertensive agents. Each animal was exposed to a single dose of every drug combination in a Latin square design.

### Methods

The following was used as the vehicle for the following study: 0.5% methylcellulose (w/v) and 0.25% polysorbate 80 (v/v) in 25 mM phosphate buffer at pH 8+/−0.2.

The following animals were used in the study: Spontaneous Hypertensive rats (Strain: SHR/NCrl). The rats were obtained from Charles River Laboratories, Inc., Kingston, New York.

The age at initiation of dosing was at approximately 12 weeks. 13 male rats were used for acclimation. 8 male rats were used for the study. The animals were identified by cage card and tattoo.

Telemetry Implantation: The animals were implanted with Data Science International transmitters (HD-S10) for collection for blood pressure and heart rate data. The animals were not administered dose formulations until at least 10 days after surgery.

Housing: The animals were individually housed in solid bottom cages equipped with water bottles.

Diet: Teklad Global Diet—Rodent 2014 (Envigo RMS, Inc.) was provided ad libitum unless otherwise specified. In some instances, the animals were fed the meal-form of this diet if indicated by health conditions.

Water: Greenfield city water was provided ad libitum.

Contaminants: No known contaminants were present in the diet, water, or bedding (if applicable) at levels that would interfere with this study.

Environment: Environmental controls for the animal room were set to maintain the following room conditions: a temperature range of 20 to 26° C., a relative humidity of 30 to 70%, and a 12-hour light/12-hour dark cycle.

Acclimation (Pre-dose Phase): The acclimation phase was for a maximum of 1 week.

Environmental and Dietary Enrichments: The animals were given various cage-enrichment devices and dietary enrichment (that do not require analyses).

Randomization: The animals were arbitrarily selected based on pre-dose phase mean arterial pressure values.

The following table shows the group designation of the rats used in the study:

TABLE 4

| | Number | | | | |
|---|---|---|---|---|---|
| | of Male | | | | |
| Group | Rats | Day 1 | Day 8 | Day 15 | Day 22 |
| 1 | 1 | Combination 1 | Vehicle | Combination 3 | Combination 2 |
| 2 | 1 | Combination 2 | Combination 3 | Vehicle | Combination 1 |
| 3 | 1 | Combination 3 | Combination 1 | Combination 2 | Vehicle |
| 4 | 1 | Vehicle | Combination 2 | Combination 1 | Combination 3 |
| 5 | 1 | Vehicle | Combination 3 | Combination 2 | Combination 1 |
| 6 | 1 | Combination 3 | Combination 1 | Vehicle | Combination 2 |
| 7 | 1 | Combination 2 | Vehicle | Combination 1 | Combination 3 |
| 8 | 1 | Combination 1 | Combination 2 | Combination 3 | Vehicle |

The following table shows the dose levels administered in the study:

TABLE 5

| | | Dose | Dose | Dose |
|---|---|---|---|---|
| | | Level | Concen- | Vol- |
| | | (mg/kg/ | tration | ume |
| Test Article | | day) | (mg/mL) | (mL/kg) |
| Combination 1 | Telmisartan | 0.17 | 0.017 | 10 |
| | Amlodipine Besylate | 0.22 | 0.022 | |
| | Indapamide | 0.08 | 0.008 | |
| Combination 2 | Telmisartan | 0.34 | 0.034 | 10 |
| | Amlodipine Besylate | 0.44 | 0.044 | |
| | Indapamide | 0.16 | 0.016 | |
| Combination 3 | Amlodipine Besylate | 0.44 | 0.044 | 10 |
| | Losartan Potassium | 0.31 | 0.031 | |
| | Hydrochlorothiazide | 1.74 | 0.174 | |
| Vehicle | 0.5% Methylcellulose | 0 | 0 | 10 |
| | (w/v), 0.25% | | | |
| | polysorbate 80 (v/v), | | | |
| | in phosphate buffer, | | | |
| | pH 8 +/− 0.2 | | | |

Dosing Procedures: For Combinations 1, 2 and 3, each test combination formulation was prepared fresh on each day of dosing. A portion of the vehicle (approximately 80%) was added to the test combination formulation and mixed until the preparations were homogenous. If a homogenous suspension or solution was not obtained, iN NaOH and/or iN HCl was added to adjust to pH 9+0.2. The remainder of the vehicle added and mixed with a stir bar. The test combination formulations were stirred continuously at room temperature and protected from light for approximately 30 minutes prior to and throughout dosing. The test combination formulations were stored protected from light, stirring in a refrigerator set to maintain a temperature of 2 to 8° C.

Dosing Procedures: For pre-dose handling, the test combination formulations were allowed to equilibrate to approximately room temperature for at least 30 minutes prior to dosing.

The animals were dosed at a volume of 10 mL/kg, and the actual dose volume was based on the most recent body weight. Oral gavage was used to administer the dose. The dose interval was once daily on Days 1, 8, 15, and 22. Following dose administration, the remaining test combination formulations were disposed of according to standard operating procedures.

Telemetry Collections: The animals were not disturbed or manipulated immediately prior to and during telemetry data collection without prior authorization. Such disturbances include but were not limited to cage changes, bedding changes, mopping, sanitation, or anything that would disturb the natural quiet environment that was important for collection of the cardiovascular telemetry data

Animal Observation: Each rat was observed once daily in the morning. Any abnormal findings were recorded. The rats were observed for mortality, abnormalities, and signs of pain or distress. Any abnormal findings that were observed during the unscheduled observation periods were also noted.

Body Weight: The body weights were obtained at least once during the pre-dose phase and prior to each scheduled dose. Additional body weights were recorded to monitor animal health if appropriate. The animals were instrumented with a transmitter: a representative transmitter and leads were used to tare the balance prior to the collection of body weights for dose calculation purposes.

Telemetry Data Collection: The arterial pressure raw signals were digitized at a sampling rate of 500 Hz. Derived parameters in the pre-dose and dosing phases were the same. For pre-dose data collection, all implanted telemetry devices were checked for consistency of signal and to verify the telemetry signal was acceptable for analysis. The signal check consisted of at least one telemetry recording taken from each rat considered for the study. Telemetry data was recorded continuously for approximately 24 hours. The telemetry data was reviewed to determine if the rat was qualified for the study. The data was maintained in the study records and was used to calculate the nominal 24-hour mean arterial pressure average to support the randomization animal selection. For dosing phase data collection, continuous telemetry data was collected during the dosing phase starting at least 90 minutes prior to dosing through approximately 48 hours post-dose.

Nominal Dosing Time: The telemetry collection time points were based on a single nominal dosing time for all animals. The nominal dosing time for each day of the dosing phase were the end of dosing for the first half of animals dosed on that day recorded on each computer for all animals on that compute.

Telemetry Data Evaluation: Telemetry parameters, including heart rate (beats/minute), systolic pressure (mmHg), diastolic pressure (mmHg), mean arterial pressure (mmHg), and arterial pulse pressure (mmHg), were analyzed and reported. Telemetry data generated by Ponemah during the dosing phases was analyzed in 1-minute samples. Data was processed in 15-minute averages and were provided for data review. The 15-minute mean data was further averaged by binning into the following Analysis Periods:
Period 1: 0.5-2 hours post-dose;
Period 2: 2-4 hours post-dose;
Period 3: 4-8 hours post-dose;
Period 4: 8-12 hours post-dose;
Period 5: 12-20 hours post-dose;
Period 6: 20-32 hours post-dose (second light cycle); and
Period 7: 32-44 hours post-dose (second dark cycle).

### Analysis

Blood pressure was measured over a 44-hour period using an implanted telemetry device. The primary outcome was systolic blood pressure.

Statistical analyses were conducted using all available data points, with weighting to reflect the non-uniform timing of measurements. Estimates of treatment effects were calculated using estimated differences between treatments using a mixed model (SAS 9.4, SAS Institute, Cary, NC) with a direct product autoregressive correlation structure to account for repeated measurements within individuals over time.

### Results

Eight animals began the study; however, the telemetry transmitter failed in one animal. As a result, complete data was available from seven animals.

The following table shows the differences in systolic BP (mmHg) between treatments:

TABLE 6

| Comparison | Level | Estimate (95% CI) | P-value |
|---|---|---|---|
| Overall difference | | | 0.0004 |
| between | | | |
| treatments | | | |
| Systolic BP (95% CI) | Control | 177.4 | — |
| | | (174.4-180.3) | |
| | Combination 1 | 172.1 | — |
| | | (169.3-174.9) | |
| | Combination 2 | 169.0 | — |
| | | (166.1-171.9) | |
| | Combination 3 | 175.4 | — |
| | | (172.5-178.3) | |
| Treatment | Control vs | −5.3 | 0.0048 |
| comparisons | Combination 1 | (−8.7-1.8) | |
| | Control vs | −8.4 | <.0001 |
| | Combination 2 | (−11.5-5.2) | |
| | Control vs | −2.0 | 0.2663 |
| | Combination 3 | (−5.6-1.7) | |
| | Combination 1 vs | −3.1 | 0.0720 |
| | Combination 2 | (−6.5-0.3) | |
| | Combination 1 vs | 3.3 | 0.0342 |
| | Combination 3 | (0.3-6.3) | |
| | Combination 2 vs | 6.4 | 0.0009 |
| | Combination 3 | (3.0-9.8) | |

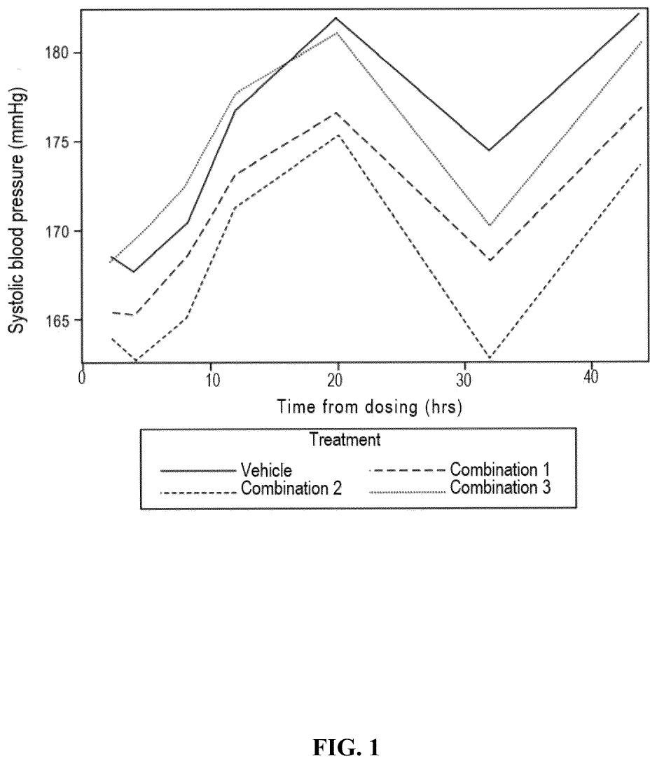

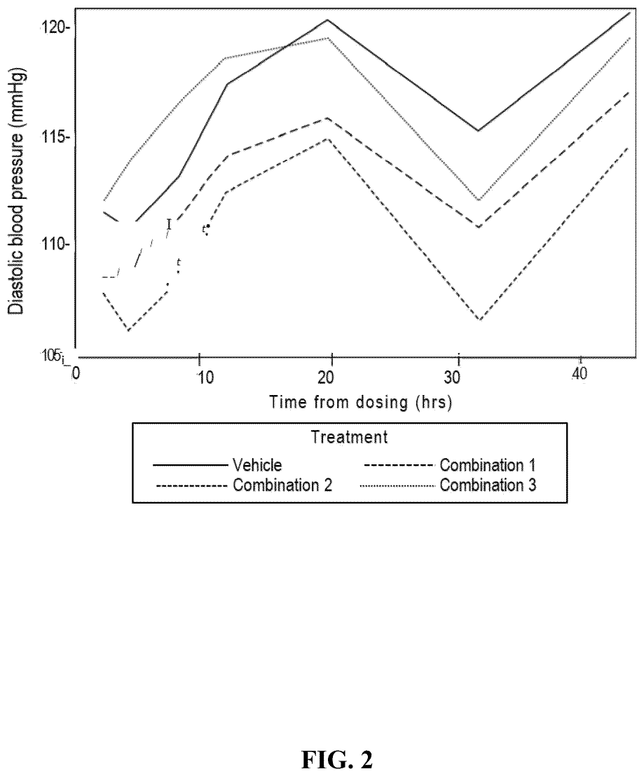

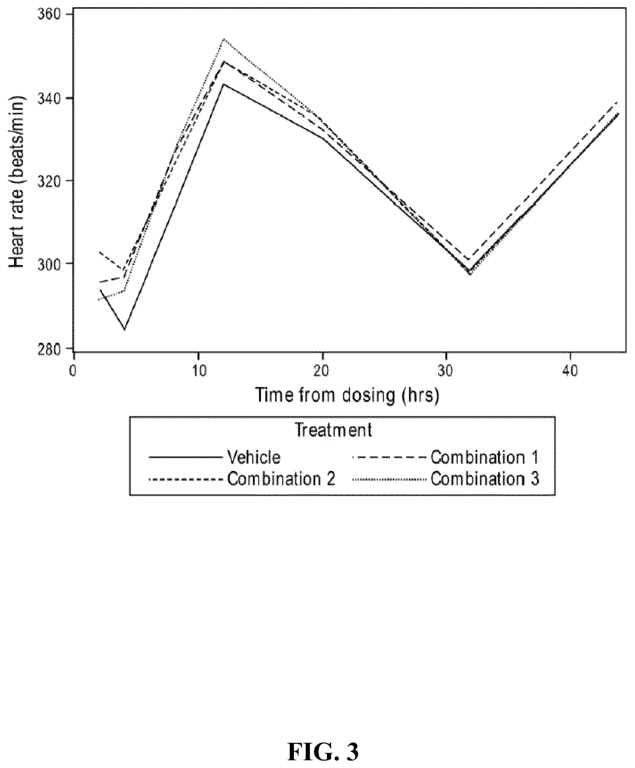

FIG. 1 shows the mean systolic blood pressure (mmHg) across time periods by treatment. FIG. 2 shows the mean diastolic blood pressure (mmHg) across time periods by treatment. FIG. 3 shows the mean heart rate across time periods by treatment.

In this non-limiting example, the results demonstrated that Combination 1 produced significantly larger reductions in systolic blood pressure than Combination 3, and Combination 2 produced significantly larger reductions in systolic blood pressure than either Combination 3 or 1. These differences persisted over the full 44-hour period of observation. The results demonstrated that both Combination 1 and Combination 2 produced significantly larger reductions in systolic blood pressure than Combination 3. These differences persisted over the full 44-hour period of observation. There were similar differences between the three combinations in the reduction in DPB, and no differences between the combinations in heart rate.

In this non-limiting example, these results demonstrate unexpected differences between the telmisartan, amlodipine besylate, and indapamide combination and the losartan, amlodipine besylate, and hydrochlorothiazide combination. Specifically, at equivalent or lower doses, the telmisartan, amlodipine besylate, and indapamide combination produced significantly larger reductions in blood pressure than the combination of losartan, amlodipine besylate, and hydrochlorothiazide. As the amlodipine besylate dose was the same in Combination 2 and 3, the results demonstrate previously unknown differences in the effectiveness of specific angiotensin II receptor blockers and specific diuretics (such as a thiazide diuretic versus a thiazide-like diuretic) when provided in parallel with amlodipine besylate.

### Example 2: Triple Combination Composition Therapy for the Treatment of Hypertension

### Methods

This study is a randomized, placebo-controlled, double-blind cross-over trial. The study is divided into three phases. During the first phase (4 weeks) participants are randomized (1:1) to either receive the triple combination composition therapy or Placebo. This is followed by a two-week washout (placebo) and subsequently participants are crossed over to the opposite arm to receive the other treatment for four weeks. Participants are recruited from the community, predominantly through community general practices in western Sydney, Australia.

### Participants

Participants are eligible if they met the following inclusion criteria: 1) adults aged 18 years and over, 2) office SBP>140 mmHg and/or DBP>90 mmHg on 2 readings on separate days; plus baseline ambulatory SBP >135 and/or DBP >85; 3) not on medical treatment for hypertension. Exclusion criteria includes: no definite contraindication to one or more component medications in the triple combination composition; the responsible clinician feels a change in current therapy would place the patient at risk; severe or accelerated hypertension; pregnancy; inability to provide informed consent; and medical illness with anticipated life expectancy less than 3 months.

### Intervention

The triple combination composition is a single encapsulated pill containing the three following components in the specified amounts: irbesartan (37.5 mg), amlodipine besylate (EQ 1.25 mg base), and hydrochlorothiazide (6.25 mg). The placebo capsule appears identical and contains placebo tablets of similar weight to those in the triple combination composition.

Participants are administered a single pill, triple combination composition or placebo, throughout the trial. Patients are instructed to take the tablets at the same time each day and are encouraged to take the tablets in the morning, but the time of the day (morning or evening) is at the patient's preference.

All trial medicines are prepared by a TGA-cGMP (Therapeutic Goods Australia—certificate of Good Manufacturing Practice) licensed manufacturing facility. Low strength doses are obtained by halving half-strength doses using a pill splitting device, without crushing, and are weighed to ensure accuracy of halving doses. The low strength doses are then encapsulated using gelatin capsules (DBCaps-Capsugel). The capsules are stored in a cool dry place and monitored using temperature loggers until they are dispensed.

Treatment allocations are blinded to both study staff and participants. In addition to the study drugs, all participants are offered education on healthier lifestyle options as recommended by guidelines for hypertension management.

### Randomization

A computer-assisted randomization sequence is generated by a statistician and supplied to the pharmaceutical packaging company. The research assistant, recruitment team, investigators are blinded to this sequence. For each patient, i.e., allocated randomization number, the pills are packaged into three child-resistant packs corresponding to the three phases of the study. All packs have identical appearance ensuring blinding of patient and research staff.

Subsequently, the medication packs are prescribed in an organized sequence.

### Outcomes and Data Collection

The primary outcome is reduction in mean 24-hour systolic blood pressure at 4 weeks using ambulatory blood pressure monitoring (ABP). The secondary outcomes include:
a. Reduction in mean 24-hour diastolic blood pressure, and in daytime and nighttime SBP and DBP at 4 weeks
b. Reduction in office SBP and DBP as measured by a standardized automated blood pressure cuff
c. Proportion with controlled blood pressure at 4 weeks, defined as <135/85 mmHg 24-hour BP and <140/90 mmHg office BP
d. Adverse events and pre-specified adverse events by laboratory parameters: Rise in transaminases (ALT/AST) more than 3× upper limit of normal or doubling if baseline levels known to be elevated; drop in estimated glomerular filtration rate by >20% as estimated from serum creatinine; sodium, potassium and uric acid levels
e. Assessment of acceptability and tolerability

Patients undergo 24-hour ABP monitoring 4 times: at baseline (off study drug), at 4 weeks (on phase 1 drug), at 6 weeks (on placebo), and at 10 weeks (on phase 3 drug). In order to minimize inconvenience, patients are referred for ABP to a lab. The ABP units are calibrated at regular intervals by the lab according to the manufacturer's specification. To minimize variability, the follow up readings are repeated from the same collection center using the same brand device. Participants are reimbursed nominal amounts to cover travel and parking costs.

Study medications and investigations are provided at no cost to participants. Office BPs are recorded three times at each visit using an OMRON T9P (HEM-759-C1). The second and the third readings are averaged for study analysis. In addition, at week 4 and 10, patients undergo blood tests to assess for biochemical side effects, are administered a questionnaire for clinical side effects, and compliance is assessed by self-report and pill count. Patients will remain blinded to their treatment allocation when completing this questionnaire.

Drug acceptability and tolerability are also assessed at the end of the study. All adverse events are recorded. In addition, clinical adverse events possibly associated with blood pressure lowering medications, such as dizziness, blurred vision, syncope/collapse, chest pain/angina, shortness of breath, cough, wheeze, pedal oedema, skin rash, and itching, are specifically asked about.

The trial has a simplified data safety and management committee of two core members with expertise in clinical medicine, trials and statistics. A single meeting convenes when 10 patients are randomized to the trial to review safety, and the study is advised to continue.

### Statistical Considerations

A sample size of 50 patients is planned to provide 90% power at p=0.05 to detect a SBP difference of 12 mmHg between the intervention and control assuming a SD of the within patient difference of 12 mmHg, taking into account the possibility of a 10% loss to follow-up.

### Statistical Approach

Analyses are conducted on an intention to treat basis. All tests are two-sided and the nominal level of a is 5%. All statistical analyses are unadjusted for prognostic covariates.

Compliance to the study drug is reported using data on pills (doses) taken and missed doses over the time period.

A linear mixed model is used to estimate the effect of the treatment on change in blood pressure from baseline for each treatment period, according to the Kenward and Roger approach (Kenward M G, Roger J H. The use of baseline covariates in crossover studies. Biostatistics 2010; 11(1): 1-17). To appropriately adjust for baseline levels collected at the beginning of each treatment period (week 0, week 6), this method uses all measurements (baseline and follow-up, in both period) as outcomes, but accounts for covariance between measurements within individuals (Liu G F, Lu K, Mogg R, Mallick M, Mehrotra D V. Should baseline be a covariate or dependent variable in analyses of change from baseline in clinical trials?Stat Med 2009; 28(20): 2509-30). A linear contrast between the variables denoting period (first/second), type of measurement (baseline/final), and treatment received (placebo/triple combination composition) produces an unbiased estimate of effect of the triple combination composition on change in blood pressure compared to the placebo. All available data are included in the model and no missing data is imputed. If a patient is missing data for one period, data from the available period is used. A sensitivity analysis is carried including only patients with data available from both periods to see if the effect of treatment is modified. There is also adjustment of the denominator degrees of freedom of Kenward and Roger (2009) that is optimal for smaller sample sizes (Kenward M G, Roger J H. An improved approximation to the precision of fixed effects from restricted maximum likelihood. Computational Statistics & Data Analysis 2009; 53(7): 2583-95).

Testing for carryover uses an unpaired t-test of the main outcome with order as an effect. Period effect is tested by using a paired t-test comparing the main outcome in period 1 with main outcome in period 2 from the same patient. A sensitivity analysis is also performed using normal paired t-test to compare primary outcome between different periods (different treatment) from the same patient, ignoring the baseline level of each period.

Continuous secondary endpoints with baseline values (e.g., daytime/night-time ambulatory SBP/DBP) are analyzed similarly to the primary endpoint. Other continuous variables without a baseline value in each period are analyzed with a paired t-test. Counts and percentages of all adverse events are reported. As a sensitivity analysis, the analyses are repeated on the complete cases (i.e., full data for each measurement period).

Tests are conducted for interaction of treatment effect with age (<=60 vs.>60 years), gender, and BMI (<=30 vs.>30 kg/m²). Subgroup analyses for each variable are also conducted. All analyses are conducted using SAS 9.4 (Cary, NC, USA) on software.

### Example 3: Comparative Study of Triple Combination Versus Standard Dose Monotherapy for the Treatment of Hypertension

### Objectives

The primary objective of this study is to investigate in a double blind randomized controlled trial whether initiating treatment with a triple combination therapy will lower blood pressure more effectively, and with fewer side effects, compared to initiating standard dose monotherapy as per current guidelines in patients with hypertension. The secondary objective is to assess if this approach is safe and has fewer side effects compared to standard care.

### Study Design

This study is a 12-week double blind randomized controlled trial (1:1) of 650 patients with grade 1 and 2 essential hypertension. Subjects are randomized through a central computer-based randomization service, to initial therapy with the triple combination composition or to an angiotensin receptor blocker (ARB), with an option to add a calcium channel blocker (CCB) as required, as per current Australian Hypertension guidelines. The primary outcome is reduction in mean systolic blood pressure using standardized automated BP cuff at 12 weeks. Secondary outcomes include: proportion with controlled blood pressure at 6 weeks, 12 weeks, ambulatory blood pressure (ABP) measures and tolerability/occurrence of adverse events.

### Eligibility Criteria

The inclusion criteria are as follows:
Adults (≥18 years).
Treatment naïve, or currently not on treatment (not taken in last 4 weeks), or taking one BP lowering drug (angiotensin converting enzyme inhibitor, angiotensin receptor blocker, calcium channel blocker, beta-blocker, aldosterone antagonist, or alpha-blocker).
SBP 140-179 mmHg and/or DBP 90-109 mmHg, documented on two occasions more than a week apart.
At least one of the measures should be documented by study staff with study automatic BP device OR recorded as daytime average SBP≥135 mmHg and/or DBP≥85 mmHg on 24-hour ambulatory BP monitoring.
At least one of these measures should be recent (in last 12 weeks).
24-hour Ambulatory BP monitoring daytime average SBP≥135 mmHg and/or DBP≥85 mmHg—documented within 12 weeks of randomization.

The exclusion criteria are as follows:
Contraindication to irbesartan, amlodipine, or indapamide.
Evidence of secondary cause of hypertension, e.g., renal artery stenosis; significant renal impairment (eGRF<50), raised serum potassium (above lab normal limit).
Women who are pregnant, breast feeding and/or of childbearing potential and not using a medically acceptable form of contraception throughout the study (pharmacological or barrier methods).
Concomitant illness, physical impairment or mental condition which, in the opinion of the study team/primary care physician, could interfere with the conduct of the study, including outcome assessments.
Participation in a concurrent interventional medical investigation or clinical trial. Patients in observational, natural history and/or epidemiological studies not involving an intervention are eligible.
Participants wherein a responsible primary care physician or another responsible physician believes it is not appropriate for participant to switch from a current monotherapy.
Inability or unwillingness to provide written informed consent.
Unable to complete study procedures including 24-hour Ambulatory BP.
Definite indication for combination therapy.

### Study Treatment

Patients who meet criteria for inclusion are randomized to: 1) A combination pill comprising the following three components: irbesartan (37.5 mg), amlodipine besylate (EQ 1.25 mg base), and indapamide (0.625 mg); or 2) irbesartan (150 mg).

Patients who are currently on monotherapy are asked to stop their treatment while they are taking the study treatment. At 6 weeks, if the BP is greater than 140/90 mmHg in either arm, amlodipine besylate (5 mg) is added by study staff.

### Outcomes

The primary outcome is the difference between groups in mean automated office systolic blood pressure at 12 weeks, adjusted for baseline values.

The secondary outcomes include the following:
The 24-hour ambulatory blood pressure measures:
   a. Difference between groups in mean 24-hour SBP and DBP at 12 weeks.
   b. Difference between groups in mean change in 24-hour SBP and DBP from 0 to 12 weeks.
   c. Difference between groups in mean daytime SBP and DBP at 12 weeks.
   d. Difference between groups in mean nighttime SBP and DBP at 12 weeks.
   e. Difference between groups in daytime, nighttime, and 24-hour BP load (percentage area under the blood pressure curve above normal day, night, and 24-hour values as per the NHFA Guide to Management of Hypertension (2008)).
   f. Difference between groups in the proportion of non-dippers (nighttime BP is not more than 10% lower than average daytime BP as per the NHFA Guide to Management of Hypertension (2008) and coefficient of variability of BP (O'Brien, E., G. Parati, and G. Stergiou, Hypertension, 2013. 62(6): p. 988-94).
Other blood pressure measures in the triple group vs control groups:
   a. Change in mean diastolic blood pressure from baseline to 12 weeks.
   b. Hypertension control (% with SBP<140 mmHg and DBP<90 mmHg) at 6 and 12 weeks.
   c. Percentage requiring step-up treatment at 6 weeks.
   d. Percentage with both BP control (as defined above) and no adverse events.
   e. Difference between groups in SBP and DBP variability.
Tolerability
   a. Difference between groups in potentially related side effects (dizziness, blurred vision, syncope/collapse/fall, chest pain/angina, shortness of breath, cough, wheeze, ankle oedema, skin rash, itching, gout, hyperkalaemia, hypokalemia, hyponatraemia, or other).
   b. Difference between groups in mean potassium, uric acid, blood glucose, cholesterol and fractions, ALT, AST, UACR (Urine albumin-to-creatinine ratio), and creatinine levels.
   c. Difference between groups in participant withdrawals from treatment.

### Statistical Methods

All analyses of study outcomes are conducted according to the principle of intention-to-treat. The primary analysis of change in systolic blood pressure (SBP) at 12 weeks is performed using an analysis of covariance (ANCOVA) including the treatment arm and baseline SBP as a covariate. Continuous secondary outcomes are analyzed similarly. Additional analyses include both 6-week and 12-week measurements in a longitudinal model including treatment arm, visit, and treatment by visit interaction, as well as the baseline measurement. Within-patient correlations are modelled using generalized estimating equations. A similar approach is applied to binary endpoints (e.g., hypertension control) with log-binomial regression used in place of linear regression. There are also pre-defined subgroup analyses, including by baseline blood pressure, gender, age, and hypertension treatment history. A detailed analysis plan is developed prior to unblinding.

### Example 4: Pharmaceutical Compositions 1

The following pharmaceutical compositions are prepared with the specified components and doses as shown in the following table.

TABLE 7

| | | |
|---|---|---|
| | Proposed | Proposed Dose |
| Agent | Dose (mg) | Range (mg) |

Composition 1

| | | |
|---|---|---|
| Amlodipine | EQ 1.25 | EQ 1 base-EQ |
| besylate | base | 1.5 base |
| Hydrochlorothiazide | 6.25 | 5-7.5 |
| Telmisartan | 10 | 8-12 |

Composition 2

| | | |
|---|---|---|
| Amlodipine | EQ 1.25 | EQ 1 base-EQ |
| besylate | base | 1.5 base |
| Indapamide | 0.625 | 0.5-0.75 |
| Telmisartan | 10 | 8-12 |

Composition 3

| | | |
|---|---|---|
| Amlodipine | EQ 1.25 | EQ 1 base-EQ |
| besylate | base | 1.5 base |
| Chlorthalidone | 12.5 | 10-15 |
| Telmisartan | 10 | 8-12 |

Composition 4

| | | |
|---|---|---|
| Amlodipine | EQ 1.25 | EQ 1 base-EQ |
| besylate | base | 1.5 base |
| Chlorthalidone | 12.5 | 10-15 |
| Irbesartan | 37.5 | 30-45 |

### Example 5: Pharmaceutical Compositions 2

The following pharmaceutical compositions are prepared with the specified components and doses as shown in the following table.

TABLE 8

| | | |
|---|---|---|
| | Proposed | Proposed Dose |
| Agent | Dose (mg) | Range (mg) |

Composition 5

| | | |
|---|---|---|
| Amlodipine | EQ 1.65 | EQ 1.5 base- EQ |
| besylate | base | 2 base |
| Hydrochlorothiazide | 8.25 | 7.5-10 |
| Irbesartan | 49.5 | 45-60 |

Composition 6

| | | |
|---|---|---|
| Amlodipine | EQ 1.65 | EQ 1.5 base- EQ |
| besylate | base | 2 base |
| Indapamide | 0.825 | 0.75-1.0 |
| Irbesartan | 49.5 | 45-60 |

Composition 7

| | | |
|---|---|---|
| Amlodipine | EQ 1.65 | EQ 1.5 base- EQ |
| besylate | base | 2 base |
| Hydrochlorothiazide | 8.25 | 7.5-10 |
| Telmisartan | 13.2 | 12-16 |

Composition 8

| | | |
|---|---|---|
| Amlodipine | EQ 1.65 | EQ 1.5 base- EQ |
| besylate | base | 2 base |
| Indapamide | 0.825 | 0.75-1.0 |
| Telmisartan | 13.2 | 12-16 |

Composition 9

| | | |
|---|---|---|
| Amlodipine | EQ 1.65 | EQ 1.5 base- EQ |
| besylate | base | 2 base |
| Chlorthalidone | 16.5 | 15-20 |
| Telmisartan | 13.2 | 12-16 |

Composition 10

| | | |
|---|---|---|
| Amlodipine | EQ 1.65 | EQ 1.5 base- EQ |
| besylate | base | 2 base |
| Chlorthalidone | 16.5 | 15-20 |
| Irbesartan | 49.5 | 45-60 |

### Example 6: Pharmaceutical Compositions 3

The following pharmaceutical compositions are prepared with the specified components and doses as shown in the following table.

TABLE 9

| | | |
|---|---|---|
| | Proposed | Proposed Dose |
| Agent | Dose (mg) | Range (mg) |

Composition K

| | | |
|---|---|---|
| Amlodipine besylate | EQ 2.5 | EQ 2 base- EQ |
| | base | 3.75 base |
| Hydrochlorothiazide | 12.5 | 10-18.75 |
| Irbesartan | 75 | 60-112.5 |

Composition L

| | | |
|---|---|---|
| Amlodipine besylate | EQ 2.5 | EQ 2 base- EQ |
| | base | 3.75 base |
| Indapamide | 1.25 | 1-1.875 |
| Irbesartan | 75 | 60-112.5 |

Composition M

| | | |
|---|---|---|
| Amlodipine besylate | EQ 2.5 | EQ 2 base- EQ |
| | base | 3.75 base |
| Hydrochlorothiazide | 12.5 | 10-18.75 |
| Telmisartan | 20 | 16-30 |

Composition N

| | | |
|---|---|---|
| Amlodipine besylate | EQ 2.5 | EQ 2 base- EQ |
| | base | 3.75 base |
| Indapamide | 1.25 | 1-1.875 |
| Telmisartan | 20 | 16-30 |

Composition O

| | | |
|---|---|---|
| Amlodipine besylate | EQ 2.5 | EQ 2 base- EQ |
| | base | 3.75 base |
| Chlorthalidone | 25 | 20-37.5 |
| Telmisartan | 20 | 16-30 |

Composition P

| | | |
|---|---|---|
| Amlodipine besylate | EQ 2.5 | EQ 2 base- EQ |
| | base | 3.75 base |
| Chlorthalidone | 25 | 20-37.5 |
| Irbesartan | 75 | 60-112.5 |

### Example 7: Methods of Treating Comprising Titrating Doses

### Background

For this study it was hypothesized that initial or early use of low-dose triple single-pill combination (SPC) incorporated into an evidence-based standardized treatment protocol could overcome many key barriers. Standardized, evidence-based treatment protocols simplify treatment, are least susceptible to treatment inertia, and streamline health system arrangements for patients with hypertension. Therefore, the objective of this trial was to evaluate, among Black African adults in Nigeria, if a low-dose triple SPC-based treatment protocol compared with the Nigeria hypertension treatment protocol is effective and safe for initial or early management of hypertension.

### Methods

### Participants

The trial was conducted in 3 public hospital-based family medicine clinics in Abuja, Kano, and Ibadan in Nigeria. Patients were recruited from those receiving care and local community referrals. Patients were eligible if they provided written informed consent, were Black African adults (self-identified) with uncontrolled hypertension (clinic systolic blood pressure [SBP]140-179 mm Hg and/or diastolic blood pressure [DBP]90-109 mm Hg), were untreated (treatment-naive or currently untreated) or receiving blood pressure-lowering monotherapy and needed initiation or intensification of therapy. Key exclusion criteria included any established cardiovascular disease, secondary hypertension, thyroid disorders, uncontrolled diabetes, kidney disease; or contraindication to any of the blood pressure-lowering drugs.

### Randomization

An independent statistician, using R statistical software (R Foundation for Statistical Computing), generated a random allocation sequence in random block sizes of 2 and 4, for central allocation of participants in a 1:1 ratio to the intervention and comparator, stratified by pre-randomization blood pressure-lowering treatment status and trial site. The random allocation sequence was concealed by incorporating it in an online system built in the electronic data capture application. Participants who satisfied the eligibility criteria were randomized by the site staff using the electronic data capture application and assigned to the allocated treatment.

### Interventions

Participants were randomly allocated to either a low-dose triple SPC-based treatment protocol or the Nigeria hypertension treatment protocol, hereafter referred to as the triple-pill protocol or standard-care protocol, respectively. The triple-pill protocol involved a new SPC which had 3 strengths (triple one-quarter, triple one-half, and triple standard doses of telmisartan/amlodipine/indapamide [i.e., 10/1.25/0.625 mg, 20/2.5/1.25 mg, and 40/5/2.5 mg]). Up-titration was via sequential doses, then addition of telmisartan (40 mg)+amlodipine (5 mg) SPC, then referral to a specialist. Tablets were taken once daily in the morning. Visits were “frontloaded,” with more initially but fewer overall. The aim was to quickly achieve a blood pressure level likely to give sustained long-term blood pressure control of less than 140/90 mm Hg: an initial visit at 2 weeks (recognizing almost all blood pressure-lowering effects are realized by this time) with up-titration if clinic blood pressure was ≥135/85 mm Hg (recognizing that mean blood pressure has to be well below 140/90 mm Hg to maintain long-term sustained control), followed by reduced visit frequency, with no visits scheduled after month 3, until month 6. Participants taking 1 blood pressure-lowering drug before randomization were asked to stop taking it before switching to the triple-pill protocol.

Participants randomized to the standard-care protocol were treated following the Federal Ministry of Health-recommended, guideline-based Nigeria hypertension treatment protocol for primary care,17 which involved the following treatment steps at monthly intervals to achieve target blood pressure less than 140/90 mm Hg: amlodipine (5 mg); then amlodipine (5 mg) and losartan (50 mg); then amlodipine (10 mg) and losartan (100 mg); then amlodipine (10 mg), losartan (100 mg), and hydrochlorothiazide (25 mg); and finally referral to a specialist if target blood pressure was still not achieved. Treatment was up-titrated if blood pressure was 140/90 mm Hg or higher at any visit.

All blood pressure-lowering drugs were provided free of charge to participants by the site staff, in sufficient amount to last until the next follow-up visit to the site. Prescribing decisions were made by a clinic physician. The triple pill was manufactured in India by a US Food and Drug Administration-approved manufacturing facility. Lifestyle advice and treatment of concomitant conditions in both of the study groups were similar.

### Measurements

The participants attended trial sites for screening, randomization (which could occur at the same visit as screening), and follow-up visits. Clinic blood pressure was measured in triplicate using a validated automatic device (Omron M6 Comfort [HEM-7321-E]; Omron Healthcare), following the method recommended by the American Heart Association (attended, with 3-5 minutes rest and 3 measures 1 minute apart).

Home blood pressure was self-measured by participants in triplicate using the same blood pressure device and measurement protocol as above, taking triplicate blood pressure measurements in the morning (before taking the trial drugs) and in the evening. To establish baseline (randomization) home blood pressure, participants were advised to measure blood pressure in the evening of the randomized day and the next morning, before starting trial medication. Thereafter, participants were advised to measure blood pressure at home on at least 4 consecutive days before each follow-up visit and on at least 1 day in other weeks. Participants were trained by the site staff on blood pressure measurement and entering values in the provided blood pressure diary. Blood and urine tests were performed at site-based laboratories at screening and at follow-up visits (months 1, 2, 3, and 6). Adherence was recorded by pill count and defined as the proportion of pills taken of pills dispensed from randomization to month 6.

### Outcomes

The primary effectiveness outcome was difference in reduction in home mean SBP from randomization to month 6, and the primary safety outcome was percentage of participants who discontinued trial treatment due to an adverse event from randomization to month 6. The main secondary effectiveness outcome was clinic blood pressure control at month 6. Clinic blood pressure control was defined as SBP less than 140 mm Hg and DBP less than 90 mm Hg, and home blood pressure control was defined as SBP less than 130 mm Hg and DBP less than 80 mm Hg. Other prespecified secondary outcomes included blood pressure control and mean blood pressure difference, for home and clinic blood pressure from randomization to months 1, 2, 3, and 6. Secondary safety outcomes covered percentage of participants with any adverse events of special interest (AESIs), serious adverse event (SAE), symptomatic hypotension, or abnormal laboratory finding from randomization to month 6. Given the well-established safety profile of studied medications, only AESIs and SAEs were collected, with the latter defined as symptomatic hypotension; abnormal levels of sodium, potassium, uric acid, or creatinine or abnormal estimated glomerular filtration rate (eGFR); headache; peripheral edema; or any other reason for trial treatment discontinuation.

The primary outcome was initially clinic blood pressure control less than 140/90 mm Hg at month 2, which was changed to difference in mean home SBP before randomization began to provide a longer-term evaluation and improved sensitivity to detect treatment effects.

### Statistical Analysis

The target sample size was 300, to provide greater than 90% power at α=0.05 to detect a home SBP difference of 6 mm Hg, assuming up to 10% loss to follow-up and a standard deviation of 11 mm Hg.

All analyses were conducted on an intention-to-treat basis. For each participant, home blood pressure at each follow-up visit was calculated by averaging all home blood pressure measurements taken between the follow-up visit and previous clinic visit scheduled for both treatment groups. Change in home blood pressure from baseline was calculated as the difference between home blood pressure at follow-up and home blood pressure at randomization. The primary outcome was estimated using a mixed-model repeated-measures (MMRM) procedure, with the dependent variable changes in home mean SBP from randomization at months 1, 2, 3, and 6, adjusted for the covariates blood pressure at randomization, age, sex (self-identified), education, and number of prior blood pressure-lowering drugs. Fixed effects included treatment group, time (as categorical), their interaction, and covariates. To account for within-participant correlation due to multiple measurements, a random participant effect with unstructured covariance was included; for the primary analysis, missing outcome values were imputed using multiple-imputations chained equations estimation. Ten imputed datasets, each obtained from 1000 iterations, were generated and analyzed using the MMRM procedure, with effectiveness estimates combined using Rubin rules. Primary effectiveness outcome sensitivity analyses were (1) estimation using observed blood pressure change from randomized to months 1, 2, 3, and 6 using multiple imputation; (2) estimation using home blood pressure measurements taken within 14 days prior and onward up to clinic visits; and (3) imputation of missing home blood pressure data using follow-up clinic blood pressure data, then estimation with the MMRM.

Risk ratio and risk differences were calculated using binomial regression with dependent variable as blood pressure control at 1, 2, 3, and 6 months and independent variables including treatment group, baseline blood pressure, and patient characteristics at baseline, age, sex, body mass index (BMI), number of blood pressure medications before randomization, and education. If models did not converge, only treatment group and baseline blood pressure were used. Multiplicity adjustment was not performed, because significance testing was conducted for 2 outcomes only and all effectiveness outcomes assessed different facets of the same underlying treatment effect on blood pressure. Subgroup analyses were pre-specified. A post hoc analysis of home blood pressure control at less than 135/85 mm Hg at month 6 was conducted, since this target is recommended by some guidelines.

### Results

### Participants

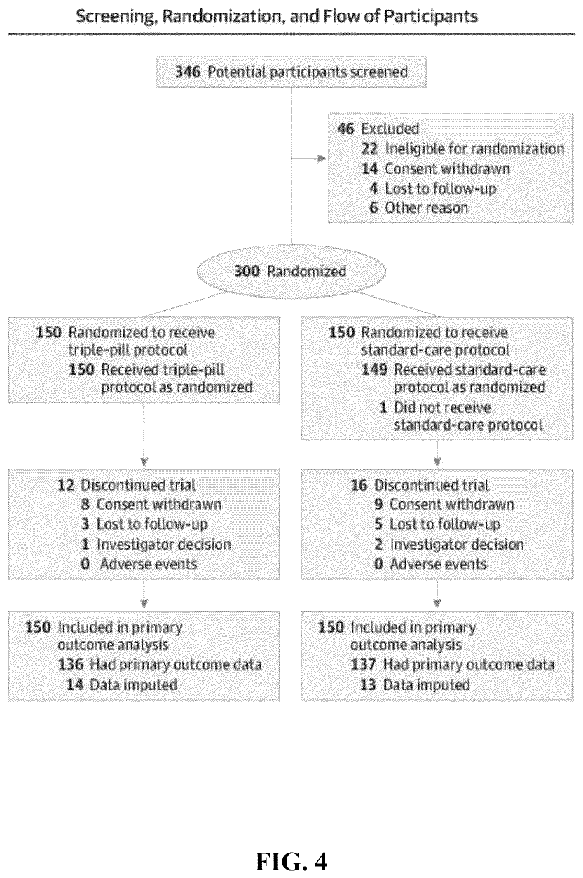

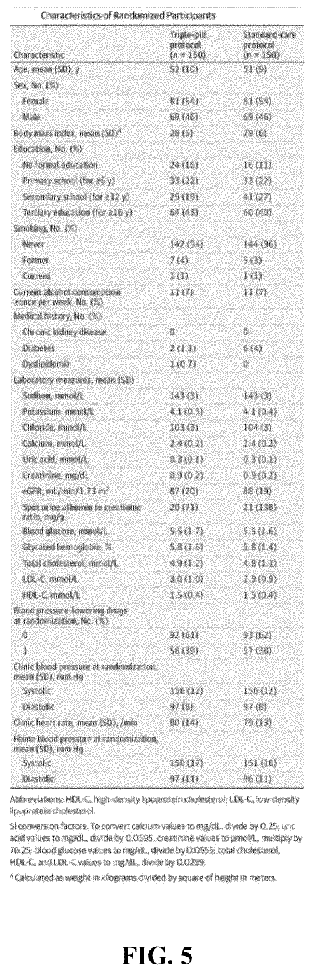

Between July 2022 to February 2024, 346 participants were screened and 300 were randomized, of whom 273 (91%) completed the trial (FIG. 4). A total of 17 (6%) participants withdrew consent, with the main reason being moving to a different location. There were no important differences in baseline characteristics between the triple therapy and dual therapy groups (FIG. 5). The mean age of the participants was 52 years, 54% were female, and 8 (3%) had diabetes. The baseline mean home blood pressure was 151/97 mm Hg and clinic blood pressure was 156/97 mm Hg. Overall, 51% had clinic blood pressure 160/100 mmHg or greater and 38% were receiving monotherapy, of whom 64% were receiving amlodipine (mostly 10 mg), 23% lisinopril, 9% nifedipine, and 4% other monotherapy. Of the 300 randomized participants, 273 (91%) had home blood pressure data at month 6.

### Effectiveness Outcomes

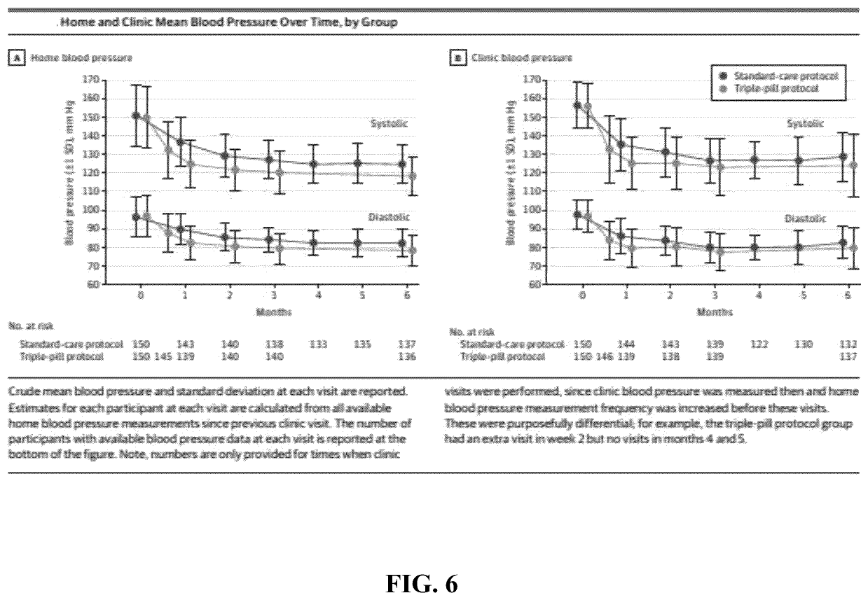

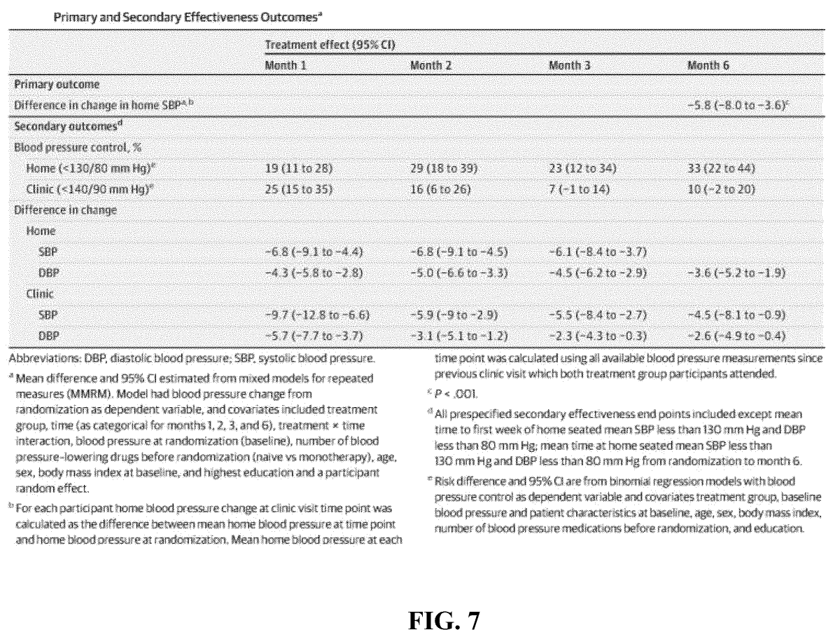

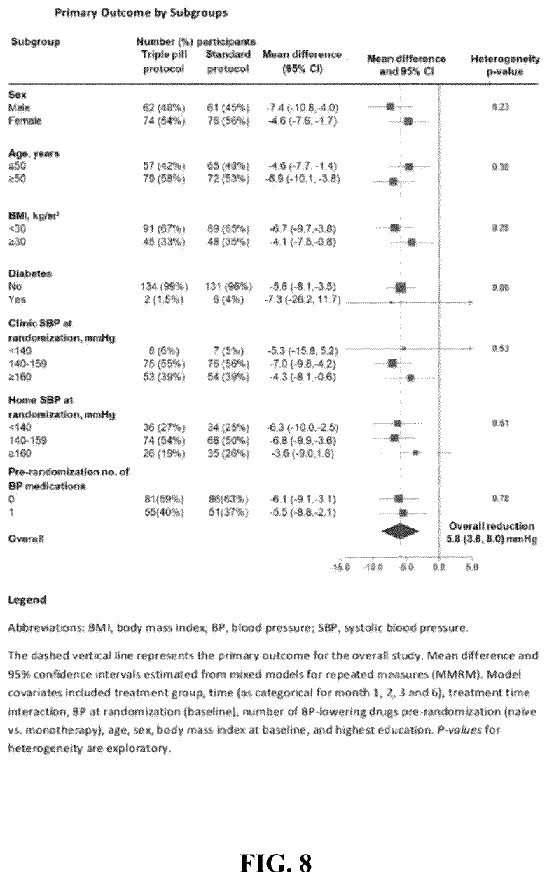

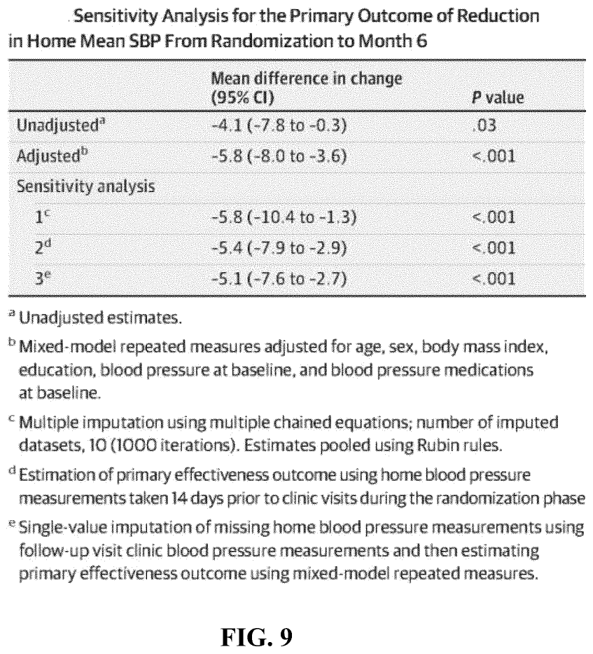

For the primary effectiveness outcome, from randomization to month 6, mean SBP was on average 31 mm Hg (95% CI, 28-33 mm Hg) lower in the triple-pill protocol group and 26 mm Hg (95% CI, 22 to 28 mm Hg) lower in the standard-care protocol group (FIG. 6). The estimated difference was −5.8 mm Hg (95% CI, −8.0 to −3.6; P<0.001) (FIG. 7). There was no evidence of heterogeneity across major subgroups (FIG. 8), and the sensitivity analyses showed that the primary outcome effect estimate was robust to a range of different assumptions (FIG. 9).

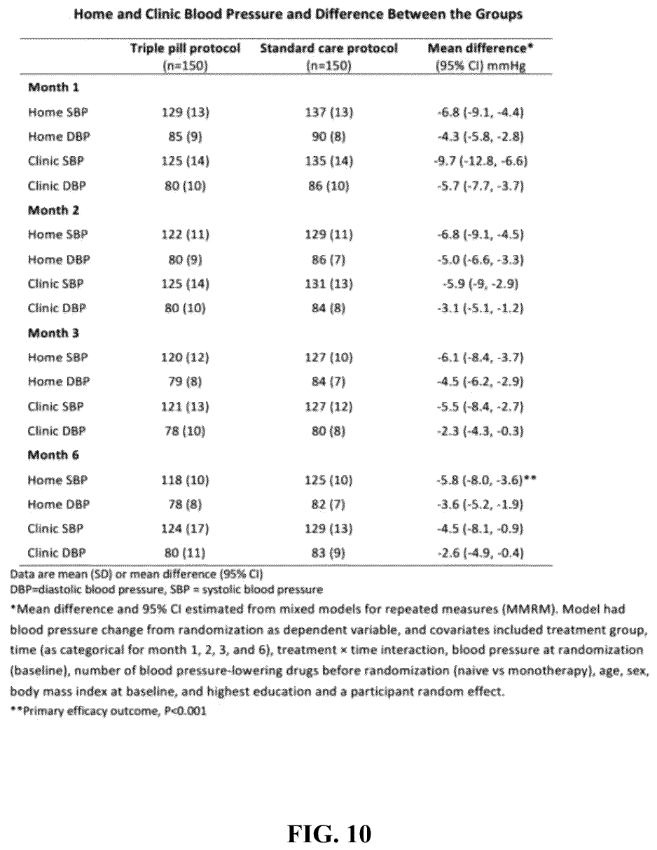

Reduction in home mean DBP from randomization to month 6 and reduction in home mean SBP and DBP from randomization to months 1,2, and 3 were all greater in in the triple-pill protocol group than in the standard-care protocol group. Clinic mean SBP and DBP reduction from randomization was greater in the triple-pill protocol group than in the standard-care protocol group at month 6, as well as months 1, 2, and 3 (FIG. 6; FIG. 10).

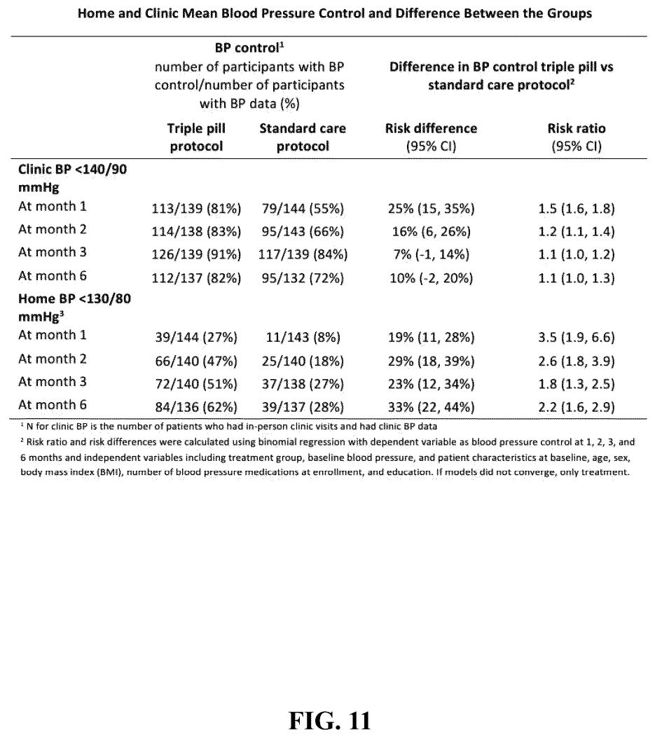

At month 6, 82% of participants in the triple-pill protocol group achieved clinic blood pressure control compared with 72% in the standard-care protocol group (relative risk [RR], 1.1 [95% CI, 1.0 to 1.3]; risk difference, 10% [95% CI, −2% to 20%]; P=0.06). At months 1, 2, and 3 follow-up visits, a greater percentage of participants achieved clinic blood pressure control in the triple-pill protocol group than in the standard-care protocol group (FIG. 7; FIG. 11). At month 6, 62% of the participants in the triple-pill protocol group achieved home blood pressure control less than 130/80 mm Hg, compared with 28% in the standard-care protocol group (RR, 2.2 [95% CI, 1.6 to 2.9]; risk difference, 33% [95% CI, 22% to 44%]). In post hoc analysis, home blood pressure control less than 135/85 mm Hg at month 6 was 82% vs 61% in triple-pill vs standard-care protocol group (RR, 1.3 [95% CI, 1.1 to 1.6]; risk difference, 20% [95% CI, 10% to 31%]).

### Safety Outcomes

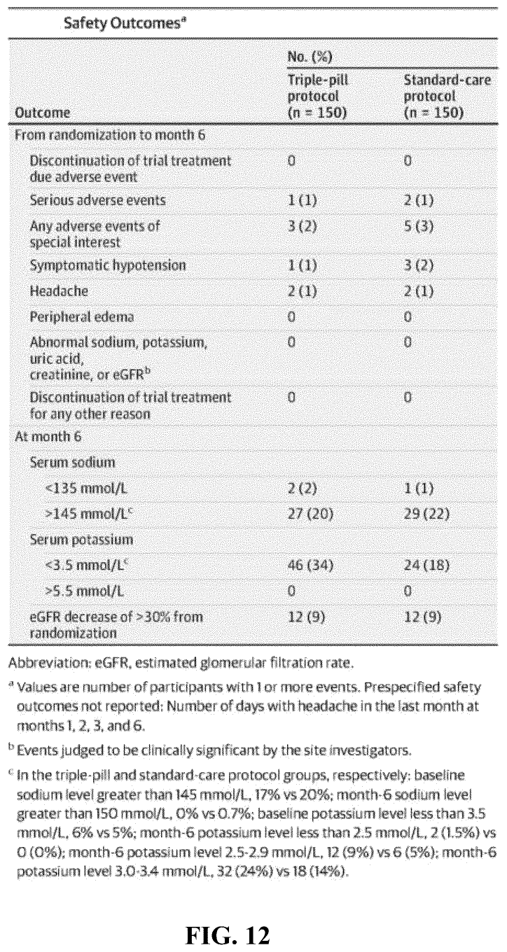

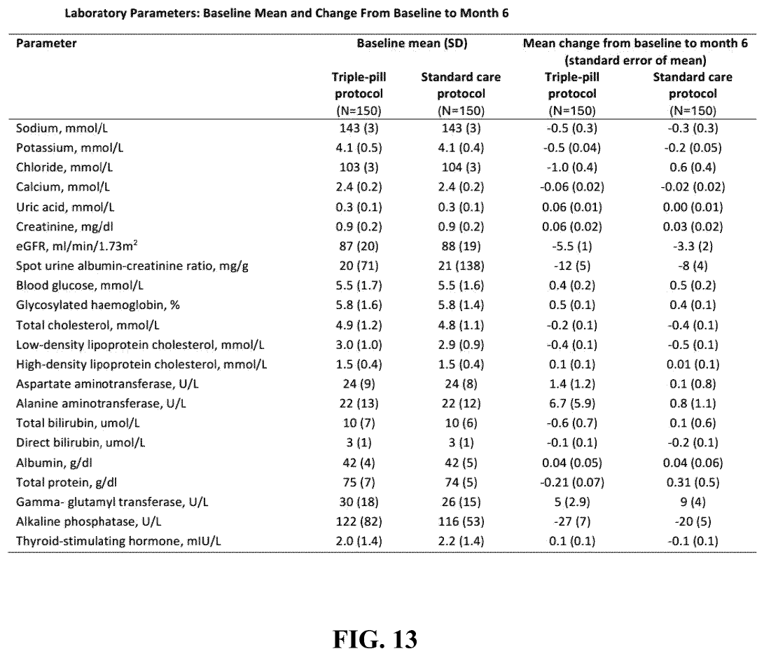

None of the participants discontinued trial treatment due to adverse events. Overall, very few AESIs were reported: 3 (2%) vs 5 (3%) participants had at least 1 AESI in the triple-pill protocol and standard-care protocol group, respectively (FIG. 12). Three participants experienced SAEs: pregnancy in the triple-pill protocol group, and fire burn and pregnancy in the standard-care protocol group. There was an excess of participants with serum potassium level less than 3.5 mmol/L at month 6 for the triple-pill vs standard-care protocol group (46 [34%] vs 24 [18%]); however, only 14 (10%) vs 6 (5%) had potassium levels less than 3.0 mmol/L at this time. Early during follow-up there was a numerical excess of participants with an eGFR decrease of greater than 30% from randomization (15 [11%] vs 6 [4%] at month 1) but at 6 months this was similar, affecting 12 (9%) in each group. No unexpected differences in laboratory parameters were observed (FIG. 13).

### Other Findings

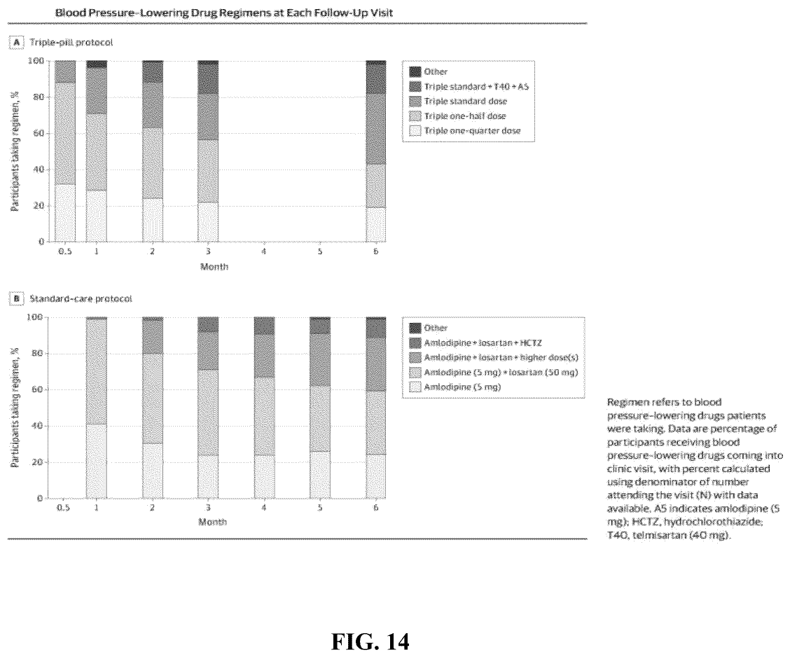

Overall, the average number of follow-up clinic visits per participant was 4.7 (SD, 1.0) in the triple-pill protocol group and 5.4 (SD, 1.4) in the standard-care protocol group. Adherence was 83% (SD, 13%) in the triple-pill protocol group vs 91% (SD, 10%) in the standard-care protocol group. At the 6-month follow-up visit, the proportion of participants receiving triple on-quarter dose was 19%; triple one-half dose, 36%; and triple standard dose, 44%. In the standard-care protocol group, the proportion of participants receiving monotherapy was 24%; dual therapy, 69%; and triple therapy, 8% (FIG. 14).

### Discussion

In this randomized trial among Black African adults with uncontrolled hypertension, a novel low-dose triple-pill protocol showed superior blood pressure reduction and similar tolerability compared with a standard care protocol. More than 80% of clinic blood pressure control was achieved in the first month and maintained over 6 months in the triple-pill protocol group.

This trial was, to Applicant's knowledge, the first to evaluate a low-dose triple SPC as initial or early therapy in Africa, and also to Applicant's knowledge the first to evaluate an accelerated up-titration schedule. These intervention components were predicated on randomized evidence on dose response and speed of action of blood pressure-lowering drugs. The trial is also one of the largest with home blood pressure outcomes in this population. In terms of previous trials, this study extends the evidence that initial or early low-dose combinations of 3 or more blood pressure-lowering drugs improve blood pressure control without increasing treatment discontinuation due to adverse events. In all trials, this strategy achieved greater than 80% blood pressure control, which was superior to usual care, despite less up-titration. Trials of dual combinations in Africa have shown 40% to 60% blood pressure control, and maximal-dose dual combinations in a very similar patient population achieved 60% to 76% blood pressure control. Collectively, these trial results and other evidence suggest that greater than 80% blood pressure control requires regimens including triple combination therapy. Tolerability of both treatment protocols was good, with no treatment-related withdrawals. The triple-pill protocol showed some initial eGFR decline (which is well recognized and reflects reversible effects of hemodynamic changes rather than intrinsic kidney damage) and hypokalemia (mostly mild). Hypokalemia rates were comparatively high in both groups, more so in the triple-pill protocol, and maybe the consequence of low dietary potassium intake in Africa.

These findings have broad clinical and public health implications, given that improved hypertension control is a priority in Africa and globally. The results underscore the need for combination therapy to be the cornerstone of effective treatment regimens. The World Health Organization (WHO) has recommended a target of 80% blood pressure control among those treated, but no country has yet achieved this goal at the national population level, even in high-income settings with few barriers to medicine availability and afford-ability. A major challenge in implementing these results in low- and middle-income countries is availability and affordability of drugs as well as integration into ongoing medicine procurement and supply chain management systems to ensure universal treatment access, even at the primary care facility level. This involves addressing structural, regulatory, pricing, and workforce barriers.

The research implications of these findings highlight the necessity for pragmatic studies to evaluate effectiveness and implementation in low- and middle-income countries, particularly in primary care, where most patients with hypertension can be diagnosed and treated. The WHO has identified cost-effectiveness of SPCs vs monotherapy as a leading research priority, along with community-based care and task-sharing. This trial was, to Applicant's knowledge, the first to test ultralow-, low-, and standard-dose options, which could be tested against a “one size fits all” strategy. Co-intervention with potassium-enriched salt substitution should be evaluated, given that it may enhance blood pressure lowering and reduce hypokalemia. Furthermore, extended dispensing intervals for SPCs are likely to improve adherence, cost-effectiveness, and supply chain efficiencies.

### CONCLUSIONS

A novel low-dose triple SPC-based treatment protocol was more effective than a standard-care protocol among Black African adults with uncontrolled hypertension, with a good tolerability profile. These benefits were shown even though the standard-care protocol achieved much better results than are normally seen in usual care and involved more visits. The results demonstrate that the WHO goal to achieve greater than 80% blood pressure control in those treated for hypertension is possible in low-income settings.

### Example 7: Dual Therapy vs. Triple Therapy Study

### Introduction

Currently available triple-drug SPCs are only indicated for substitution among individuals already taking all the three component drugs, or among those with inadequate blood pressure control on two of the component drugs. Furthermore, there are no existing SPC products with low doses of an angiotensin-II receptor blocker, a calcium channel blocker, and a thiazide-like diuretic. To address this unmet need, two triple SPC products of telmisartan, amlodipine, and indapamide were developed. One composition had telmisartan 20 mg, amlodipine 2.5 mg, and indapamide 1.25 mg (hereinafter, “Composition A”) and one composition had telmisartan 40 mg, amlodipine 5 mg, and indapamide 2.5 mg (hereinafter, “Composition B”). One crucial aspect of developing SPCs is to assess the contribution of each component drug to efficacy and safety. A trial was therefore conducted among adults with hypertension to compare Compositions A and B with each of the three dual components (i.e., combinations of telmisartan-indapamide, telmisartan-amlodipine and amlodipine-indapamide) for blood pressure-lowering efficacy and safety.

### Methods

### Study Design

In this international, randomised, double-blind, active-controlled, parallel-group trial, following a 4-week single-blind, active run-in, eligible participants were randomly allocated to a 12-week treatment period to investigate the efficacy and safety of Composition A compared with dual combinations of the component drugs in adults with high blood pressure. Participants were recruited from 83 clinics or hospital-based outpatient departments or primary care centers that provide hypertension care in Australia, the Czech Republic, New Zealand, Poland, Sri Lanka, the UK, and the USA.

Participants were eligible if they were aged 18 years or older, had been diagnosed with hypertension, and provided signed consent to participate. Additionally, at the screening visit, clinic systolic blood pressure (SBP) had to be 140-179 mm Hg on no blood pressure-lowering drugs, 130-170 mm Hg on one blood pressure-lowering drug, 120-160 mm Hg on two blood pressure-lowering drugs, or 110-150 mm Hg on three blood pressure-lowering drugs.

Following the 4-week active run-in on Composition A, participants were eligible for randomisation if their home SBP in the preceding week was 110-154 mm Hg, their adherence to the run-in medication was 80-120%, the treatment was tolerated, and the participants adhered to their home blood pressure monitoring schedule. During the first 15 months of trial conduct, the home SBP range for eligibility was 120-154 mm Hg, but the protocol was amended to allow an SBP of 110-154 mm Hg given the high proportion of individuals with SBP levels below 120 mm Hg during their run-in. Exclusion criteria included: treatment with four or more antihypertensive drugs, or use of antihypertensive drugs for indications other than hypertension (e.g., heart failure); contra-indication to the study medications; history of any established cardiovascular disease, uncontrolled diabetes, or kidney disease (e.g., estimated glomerular filtration rate [eGFR]<60 mL/min per 1.73 m²); a known secondary cause of hypertension; childbearing potential; factors that would adversely affect trial participation, such as a physical or mental health condition, night shifts, or history of alcohol or drug abuse during the preceding 12 months; or an arm circumference that was too large or too small for the available blood pressure measurement cuffs.

### Randomization and Masking

After a screening visit, eligible participants were switched from existing antihypertensive drugs to run-in on Composition A half dose. Following the run-in, eligible participants were randomly allocated (2:1:1:1) to continue Composition A or to receive telmisartan 20 mg with indapamide 1.25 mg, telmisartan 20 mg with amlodipine 2.5 mg, or amlodipine 2.5 mg with indapamide 1.25 mg in a double-blind manner using capsules with an identical appearance for 6 weeks.

At the week 6 visit, all of the doses were doubled. In other words, patients who had continued taking Composition A following the run-in were switch to Composition B. Patients taking 20 mg of telmisartan with 1.25 mg of indapamide were switched to telmisartan 40 mg with indapamide 2.5 mg. Patients taking 20 mg of telmisartan with 2.5 mg of amlodipine were switched to telmisartan 40 mg with amlodipine 5 mg. Patients taking 1.25 mg of indapamide with 2.5 mg amlodipine were switched to amlodipine 5 mg with indapamide 2.5 mg. These groups were maintained for a further 6 weeks, unless the investigator believed there was a specific contraindication for a particular participant, such as symptomatic hypotension or a very low home or clinic blood pressure (e.g., SBP<100 mm Hg).

Participants were advised to take one capsule of trial medication in the morning at approximately the same time each day (either before or after breakfast), immediately after taking their morning home blood pressure measurements. A random-sequence treatment allocation procedure was incorporated into an online electronic data capture application by the unmasked study statistician. Neither the investigators nor site staff had access to the randomisation sequence. Participants meeting the eligibility criteria for randomisation were randomly allocated in the online electronic data capture application. The application generated the randomisation record with the participant identification number, date, and time. After completion of the double-blind treatment period at week 12, participants were switched to non-trial medication as per local guidelines and practice.

### Procedures

From the beginning of the run-in period until the end of the double-blind period, participants measured their blood pressure at home, following procedures outlined in printed instructions informed by American Heart Association recommendations (Rodgers A, Salam A, Cushman W, et al. Rationale for a new low-dose triple single pill combination for the treatment of hypertension. Glob Heart. 2024; 19(1):18. doi:10.5334/gh.1283, which is incorporated herein by reference in its entirety” with reference to recent trials and clinical use given to the participants (Lasserson D S, Buclin T, Glasziou P. How quickly should we titrate antihypertensive medication?systematic review modelling blood pressure response from trial data. Heart. 2011; 97(21):1771-1775; Egan B M, Li J, Wagner C S. Systolic Blood Pressure Intervention Trial (SPRINT) and target systolic blood pressure in future hypertension guidelines. Hypertension. 2016; 68(2):318-323, which are incorporated herein by reference in their entirety). Each participant was supplied with a FORA D40 g blood pressure machine (also known as Medisanté BP800 machine; Taidoc Technology, New Taipei City, Taiwan), which is a validated, electronic, automatic, digital upper-arm cuff monitor. Blood pressure readings were encrypted and transferred automatically to the trial database over the Global System for Mobile communication cellular network. Home blood pressure was to be measured: on four consecutive days immediately preceding a trial visit, and once a week on other weeks; in triplicate in the morning and in the evening; and in the morning immediately before the next trial medication dose. Blood pressure was measured with the participant in a seated position during all scheduled trial visits, using the same machine and a standard procedure, specified in the protocol and instruction manual.

### Outcomes

The primary efficacy outcome was difference between the triple combinations and the dual combinations in home seated mean SBP change from randomisation to week 12. At the outset of the trial, the primary outcome was clinic blood pressure, but this was switched, before randomisation began, to home blood pressure at the beginning of the COVID-19 pandemic. Secondary efficacy outcomes were differences in clinic blood pressure and home diastolic blood pressure (DBP) changes, and proportion of participants with blood pressure control (clinic blood pressure <140/90 mm Hg and <130/80 mmHg; home blood pressure <135/85 mm Hg and <130/80 mm Hg, including at trough), at weeks 6 and 12. The primary safety outcome was percentage of participants discontinuing trial medication due to an adverse event from randomisation to week 12. The secondary safety outcomes were proportion of participants discontinuing trial medication due to an adverse event from randomisation to week 6, and proportion of participants with serious adverse events, symptomatic hypotension, hyponatraemia or hyper-natraemia, hypokalaemia or hyperkalaemia abnormalities, eGFR drop of more than 30%, or orthostatic hypotension or hypertension at weeks 6 and 12. Other than serious adverse events, only data on adverse events of special interest were collected. An adverse event of special interest was defined as the following set of adverse events: symptomatic hypotension; abnormal laboratory findings of sodium, potassium, uric acid, glucose, lipids, creatinine, or eGFR; headache; peripheral oedema; or any other symptom or laboratory abnormality that led to permanent discontinuation of trial medication.

### Statistical Analysis

A total of 1385 randomly assigned participants provided more than 97% power to detect a minimum clinically significant difference of 3 mm Hg in home mean SBP for each of the three comparisons of triple therapy versus dual therapy, assuming an analysis of covariance-type approach, a common SD of home SBP of 11 mm Hg, and a correlation coefficient of 0.4. The overall power for all three comparisons was therefore more than 90% (0.973=0.91). A sample size of 1500 was originally planned. However, continued supply of study treatment became unfeasible due to COVID-19 pandemic-related delays; a review of study power, based on the prespecified statistical approach and a blinded assessment of the SD of the overall sample, indicated that 1385 participants would have sufficient statistical power. All tests were two-sided with a nominal level of a set at 5%. Because the purpose of the trial was to show effects on all three of the designated primary efficacy endpoint comparisons simultaneously (i.e., superiority was required for all three triply therapy vs dual therapy comparisons for the trial to be regarded as positive), there was no need for adjustment of the type I error for the primary endpoint.

Additionally, the secondary efficacy parameters measured different aspects of the same underlying treatment effect, that is, the effect of triply therapy compared with dual combinations on blood pressure, and do not show additional new treatment effects of the drug but rather clarify the effect already shown by the primary analysis. Therefore, no type I error adjustment was conducted to account for multiplicity for secondary efficacy endpoint analyses.

An independent data and safety monitoring board had responsibility for safeguarding the interests of participants by reviewing interim safety and efficacy data. The estimand framework was used, with a treatment policy strategy as a primary estimand (Muntner P, Shimbo D, Carey R M, et al. Measurement of blood pressure in humans: a scientific statement from the American Heart Association. Hypertension. 2019; 73(5):e35-e66, which is incorporated herein by reference in its entirety), which most closely aligns to an intention-to-treat analysis in which all available and imputed data contribute towards the estimated treatment effects, irrespective of intercurrent events. The population for the primary estimand included adults with hypertension, the variable of interest was difference between baseline and week 12 home SBP, and the primary analysis was performed on the randomised set, which included all participants who were randomly assigned to treatment. Intercurrent events (that is, post-randomisation events that affect either the interpretation or existence of outcome data) were defined ahead of data lock in the statistical analysis plan.

Baseline characteristics by treatment group were summarised descriptively. To calculate home blood pressure averages, the first measurement from each of the home blood pressure triplicates was dropped and the remaining measurements were averaged for each participant. The primary analysis was performed using a mixed model with repeated measures, including week 6 and week 12 measurements with baseline blood pressure, visit, treatment group, and visit by treatment group interaction as fixed effects, accounting for correlation within participants and clustering at the site level and variance estimated using a Huber-White sandwich estimator. For participants with missing primary outcome data, a Retrieved Data Multiple Imputation approach was employed (found at Pant R, Salam A, Rodgers A. Evaluation of a triple single-pill combination (triple pill)-based treatment protocol compared to the Nigeria hypertension treatment protocol for improving blood pressure control in Nigeria-statistical analysis plan. Published 2024. Accessed Aug. 16, 2024; and Brown M J, Williams B, Morant S V, et al; British Hypertension Society's Prevention and Treatment of Hypertension with Algorithm-based Therapy (PATHWAY) Studies Group. Effect of amiloride, or amiloride plus hydrochlorothiazide, versus hydrochlorothiazide on glucose tolerance and blood pressure (PATHWAY-3): a parallel-group, double-blind randomised phase 4 trial. Lancet Diabetes Endocrinol. 2016; 4(2):136-147, which are incorporated herein by reference in their entirety) with missing data imputed only from participants who were concordant with presence or absence of an intercurrent event. A total of 100 imputed datasets were used. Prespecified sensitivity and supplementary analyses included: use of only week 12 home blood pressure results; analysis adjusted for covariates; analysis after excluding participants with study treatment interruptions for supply-related reasons, complete cases with clinic blood pressure substitution for missing home blood pressure values; and a per-protocol analysis, excluding any participants with an intercurrent event or major protocol deviation. A two-dimensional multiple-imputation tipping point analysis was conducted to evaluate the effect of missing data under the assumption of data not missing at random. This involved imputing missing week 12 data for both the Composition A and active control groups, applying a shift parameter to assess when statistical significance was lost (p>0.05) for at least one comparison. Other continuous outcomes of difference in change in blood pressure were analysed as per the primary outcome. All continuous outcomes were reported along with 95% CI and the corresponding p value. The proportion of participants with blood pressure control was descriptively summarised and analysed using generalised estimating equations with the visit, treatment group, and visit by treatment group interaction as fixed effects and accounting for correlation within participant and clustering at the site level. Proportions by treatment group with 95% CI were presented along with the associated estimated risk difference and its corresponding p value. Other binary outcomes of efficacy and safety were analysed as per the percentage of participants with blood pressure control. Statistical analyses were conducted with SAS version 9.4 and R software.

### Results

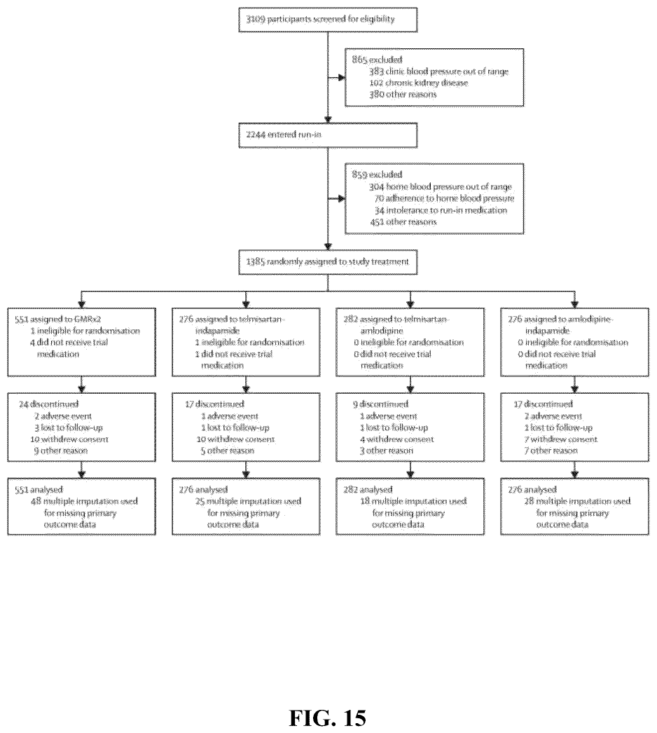

The trial was conducted from Jul. 9, 2021, to Sep. 1, 2023. Trial conduct was affected by the COVID-19 pandemic in multiple ways, including slow enrolment and interruptions in recruitment due to issues with trial medication supply. A decision was made at the start of the trial for all episodes of COVID-19 to be classified as serious adverse events, but not all met the usual criteria for severity. 3109 participants were screened, of whom 2244 (72.2%) entered run-in and 1385 (44.5%) were randomly assigned (551 to Composition A, 276 to telmisartan-indapamide, 282 to telmisartan-amlodipine, and 276 to amlodipine-indapamide), with the main reasons for screening and run-in failure related to blood pressure criteria (FIG. 15). During the single-blind run-in with the Composition A, 25 (1%) participants had a serious adverse event, of which 18 were due to COVID-19, and only one due to increased uric acid was judged to be related to the study treatment. 72 (3%) participants discontinued trial treatment due to an adverse event.

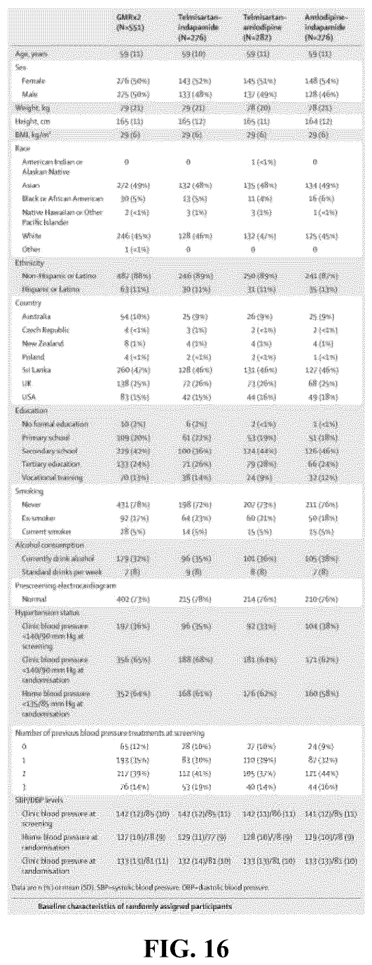

The mean age of randomly assigned participants was 59 years (SD 11); 712 (51.4%) self-reported as female, and 673 (48.6%) as male. Baseline characteristics were similar across the groups (FIG. 16). Mean screening clinic blood pressure for the randomly assigned participants, who were receiving an average of 1.6 blood pressure medications, was 142/85 mm Hg; and after the 4-week run-in average clinic blood pressure was 133/81 mm Hg and home blood pressure was 129/78 mm Hg. All but five eligible participants commenced randomised treatment, and at week 6 the proportion undergoing dose doubling was 442 (80%) of 551 in the Composition A, 221 (80%) of 276 in the telmisartan-indapamide, 242 (86%) of 282 in the telmisartan-amlodipine, and 233 (84%) of 276 in the amlodipine-indapamide group. Of the 1385 participants who were randomly assigned, 1318 (95.2%) completed follow-up. Adherence, defined as the proportion of pills planned for the randomised phase that were taken, was 96% overall: 78 (95%) of 82 for Composition A, 79 (98%) of 81 for telmisartan-indapamide, 79 (96%) of 82 for telmisartan-amlodipine, and 79 (95%) of 83 for amlodipine-indapamide.

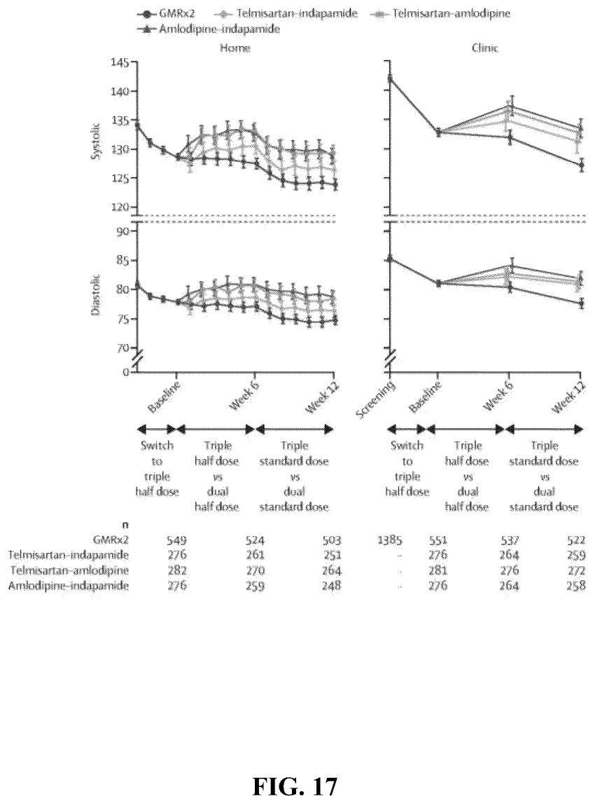

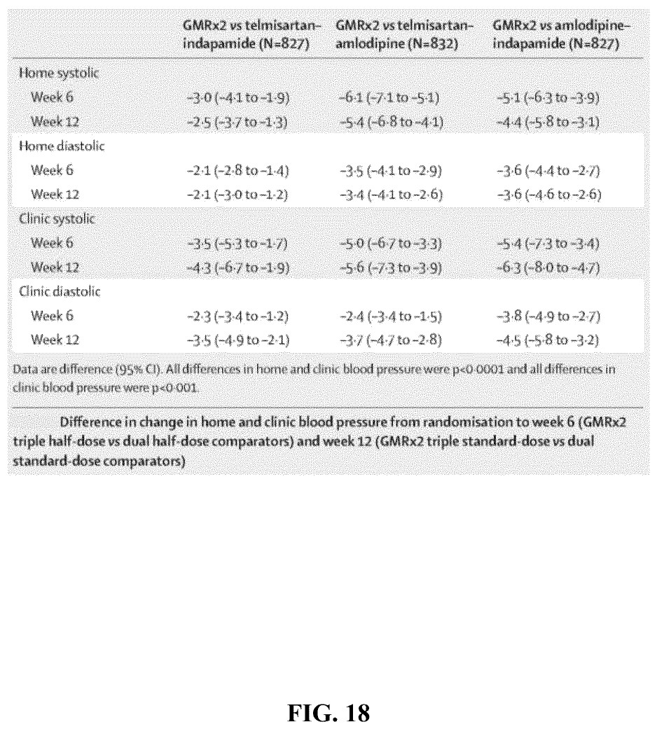

At 12 weeks, the primary outcome (home SBP) was lower in the triple therapy (Composition B) group compared with each of the dual-therapy groups: the least-squares differences in change in home seated mean SBP mm Hg from randomisation to week 12 was −2.5 (95% CI −3.7 to −1.3, p<0.0001) for Composition B versus telmisartan-indapamide, −5.4 (−6.8 to −4.1, p<0.0001) versus telmisartan-amlodipine, and −4.4 (−5.8 to −3.1, p<0.0001) versus amlodipine-indapamide (FIG. 17, FIG. 18). The findings were not materially altered in all sensitivity analyses, including a tipping point analysis. Findings were broadly consistent across predefined subgroups. Although there was statistically significant heterogeneity within subgroups defined by number of blood pressure medications at screening (triple therapy vs telmisartan-indapamide), home SBP at randomization (triple therapy vs telmisartan-amlodipine), and BMI and region (triple therapy vs amlodipine-indapamide), these findings should be considered in light of the 11 separate subgroup analyses conducted for each of the three triple therapy versus dual therapy comparisons.

Reductions in clinic SBP and DBP were also seen for all comparisons of triple therapy versus dual combinations at each timepoint, and on average these were about 20% greater than those for home blood pressure. There was also broad consistency in the reductions in clinic SBP across different subgroups. For both clinic and home blood pressure, there was no clear difference between the size of the additional blood pressure reduction conferred by the triple therapy half dose versus dual half doses (i.e., Composition A versus corresponding dual therapies) and between the standard dose versus dual standard doses, at least for home blood pressure (i.e., Composition B versus corresponding dual therapies) (FIG. 18). For example, the average blood pressure differences for Composition A versus dual half doses at week 6 were 4.7/3.1 mm Hg for home blood pressure and 4.6/2.8 mm Hg for clinic blood pressure, and for Composition B versus dual standard doses at week 12 they were 4.1/3.0 mm Hg for home blood pressure and 5.4/3.9 mm Hg for clinic blood pressure.

The least-squares difference in change in trough home mean SBP mm Hg from randomization to 12 weeks was −1.9 (95% CI −3.2 to −0.6, p<0.0043) for Composition A versus telmisartan-indapamide, −5.6 (−7.0 to −4.2, p<0.0001) versus telmisartan-amlodipine, and −3.7 (−5.2 to −2.3, p<0.0001) versus amlodipine-indapamide, indicating effects were maintained at the end of the dosing interval. Composition A also conferred similar-sized reductions compared with each dual therapy in week 6 for home SBP, and there were also reductions in home DBP for all comparisons at both visits (all p<0.001; FIG. 18).

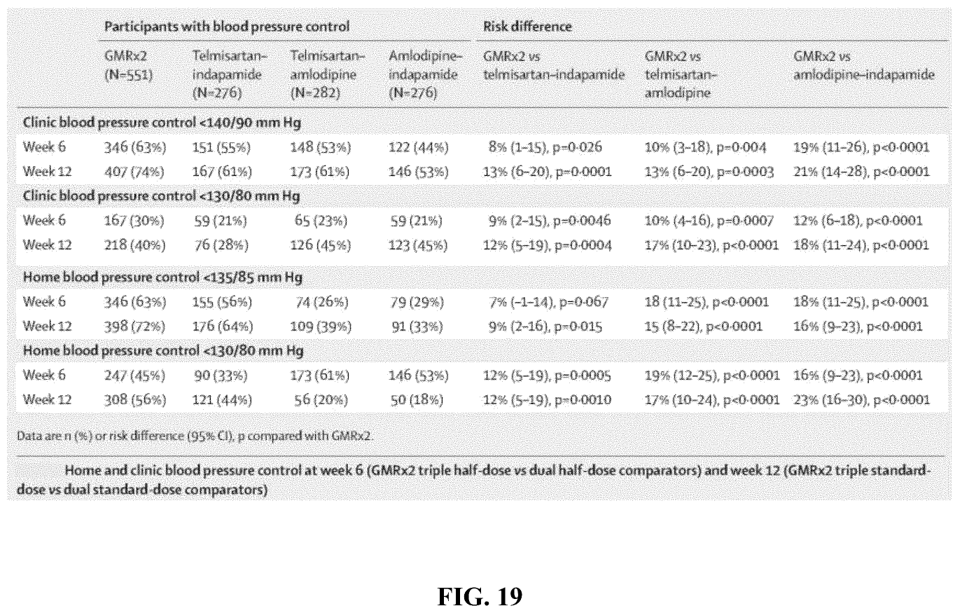

Improvements in blood pressure control rates were seen for all triply therapy versus dual therapy comparisons, at each timepoint, for home and clinic measures and for different threshold definitions (FIG. 19). For example, the proportions of participants with clinic blood pressure below 140/90 mm Hg at week 12 were 407 (74%) of 551 for triple therapy, 167 (61%) of 276 for telmisartan-indapamide, 173 (61%) of 282 for telmisartan-amlodipine, and 146 (53%) of 276 for amlodipine-indapamide; the absolute differences were 13% (95% CI 6-20, p=0.0001) for triple therapy versus telmisartan-indapamide, 13% (6-20, p=0.0003) for triple therapy versus telmisartan-amlodipine, and 21% (14-28, p<0.0001) for triple therapy versus amlodipine-indapamide. The size of the absolute improvement in various blood pressure control rates was broadly consistent, with point estimates of higher blood pressure control rates in triple therapy versus comparator groups ranging from a 7% to a 23% absolute increase.

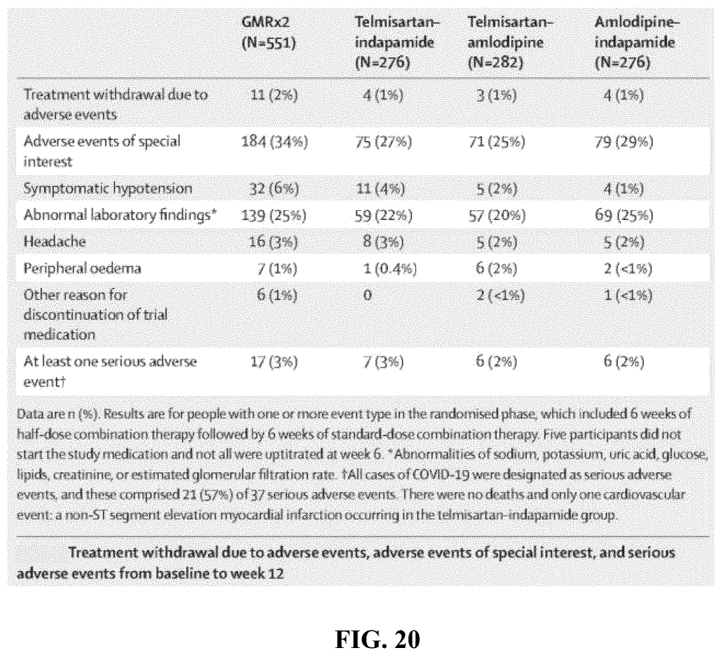

There was no statistically significant difference in the primary safety outcome of discontinuation of study treatment due to adverse events from randomization to week 12. The absolute rates of discontinuation were 11 (2%) for triple therapy, four (1%) for telmisartan-indapamide, three (1%) for telmisartan-amlodipine, and four (1%) for amlodipine-indapamide; the absolute differences were 0.6% (95% CI −2.1 to 2.5) for triple therapy versus telmisartan-indapamide, 0.9% (−1.5 to 2.8) for triple therapy versus telmisartan-amlodipine, and 0.6% (−2.1 to 2.5) for triple therapy versus amlodipine-indapamide (FIG. 20).

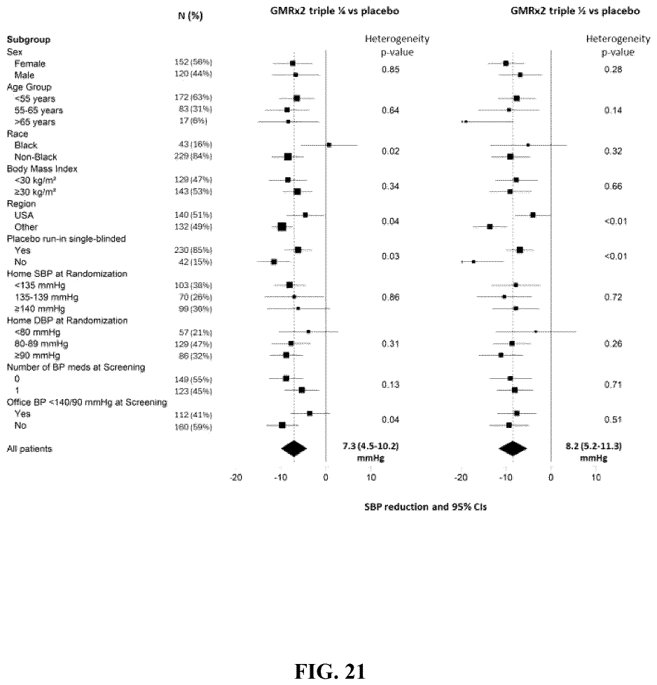

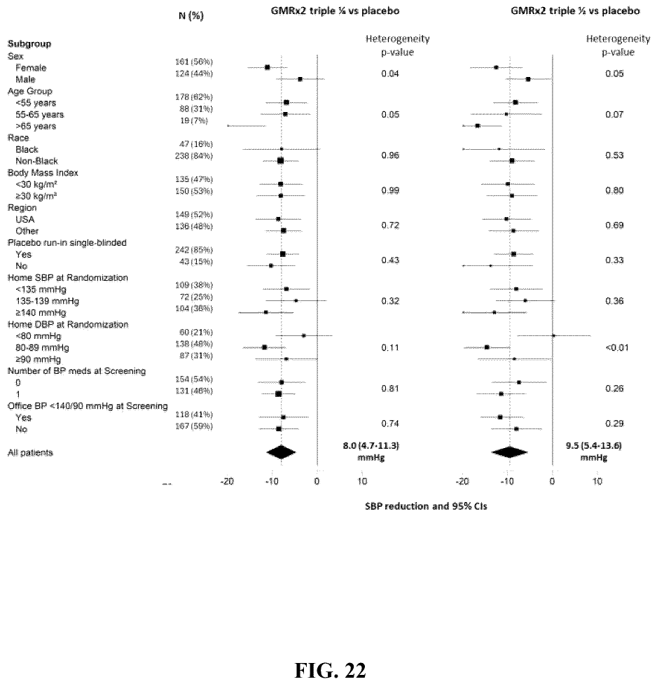

17 (3%) participants in the triple therapy, seven (3%) in the telmisartan-indapamide, six (2%) in the telmisartan-amlodipine, and six (2%) in the amlodipine-indapamide group had a serious adverse event (FIG. 20). No deaths were reported during the study. 32 (6%) participants had symptomatic hypotension in the triple therapy group compared with 11 (4%) in the telmisartan-indapamide, five (2%) in the telmisartan-amlodipine, and four (1%) in the amlodipine-indapamide group at week 12. Similarly, at week 6, 15 (3%) participants had symptomatic hypotension in the triple therapy, one (less than 1%) in the telmisartan-indapamide, three (1%) in the telmisartan-amlodipine, and two (less than 1%) in the amlodipine-indapamide group. FIGS. 21 and 22 show further results, broken down by demographics, from the study. FIG. 21 shows the primary efficacy outcome (difference in change in home systolic blood pressure) within different demographic groups. FIG. 22 shows the secondary efficacy outcome (difference in change in clinic SBP at week 4) within different demographic groups.

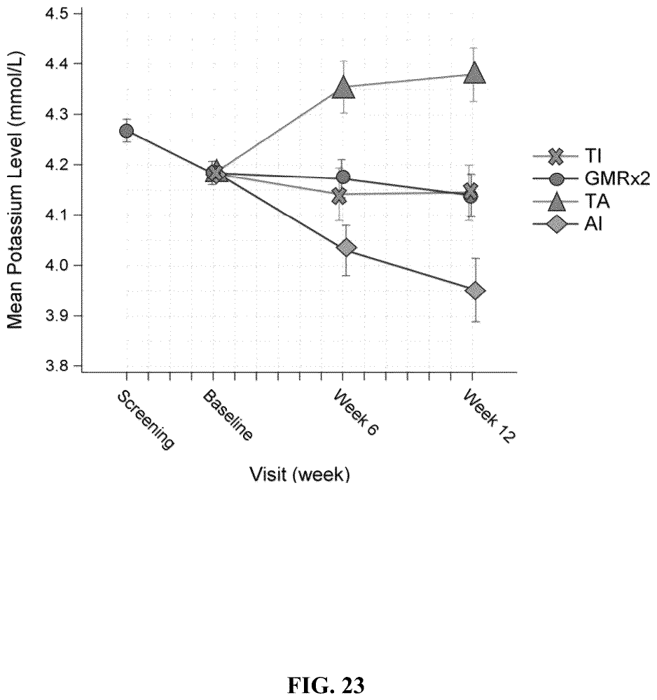

There were no between-group differences in measures of eGFR. Out-of-range sodium or potassium values occurred more frequently in the triple therapy group compared with the telmisartan-amlodipine group (19.1% vs 8.5%, risk difference 10.6% [95% CI 5.4-15.1]) at week 12, but rates for the triple therapy group were similar to those seen in the two dual-combination groups that included indapamide (FIG. 23). Very few clinically significant electrolyte abnormalities occurred in any groups. There was no significant difference in measures of orthostatic hypotension or hypertension between the groups when measured in clinic visits at week 6 or week 12.

### Discussion

To the Applicant's knowledge, this trial was the first to assess the contributions of telmisartan, amlodipine, and indapamide as part of a triple half-dose as well as triple standard-dose combination therapy in hypertension. The trial assessed efficacy at lower levels of blood pressure than previous studies and showed that triple therapy was more effective than dual therapy, significantly reducing both home and clinic blood pressure and improving blood pressure control at both half and standard doses. Tolerability was good, with no increase in withdrawal due to adverse events.

Standard or maximal dose triples on average conferred a 5.1/3.7 mm Hg additional SBP/DBP reduction compared with dual therapy. The relevance of the smaller difference between triple therapy and a renin angiotensin system-diuretic dual combination for home blood pressure is uncertain, given this was not clearly seen for clinic SBP in this or previous triple-combination versus dual-combination trials or expected from other large trials comparing dual combinations. Similarly, the relevance of the subgroup findings for home blood pressure are uncertain given the number of subgroup analyses conducted and inconsistency for home and clinic blood pressure; for example, the lowest p value for heterogeneity for home SBP analyses suggested lesser reduction with BMI lower than 30 kg/m2 versus 30 kg/m2 or higher for the triple pill protocol versus amlodipine-indapamide comparison, but the same comparison with clinic SBP showed a numerically greater reduction for those with BMI lower than 30 kg/m2. The tolerability findings are also broadly consistent for the standard-dose comparisons, with around a 1% absolute excess observed in this trial compared with a 2% excess in past trials but overlap in CIs.

Strengths of this trial included randomisation, double blinding, and enrolment of enough participants to provide adequate statistical power to assess the treatment effects on home as well as clinic blood pressure in a broad array of participants with hypertension, including those taking between zero and three antihypertensive drugs at baseline. The trial provided evidence at blood pressure levels lower than those studied in previous trials, for which baseline blood pressure levels averaged 169/103 mm Hg among untreated populations and 150/95 mm Hg for those uncontrolled on dual antihypertensive therapy. In contrast, our trial mean clinic blood pressure was 133/81 mm Hg at baseline and 142/85 mm Hg when participants began a triple half-dose run-in. This contrast means the trial generated evidence on efficacy and tolerability at blood pressure levels of current interest, given the increasing guideline recommendations for blood pressure targets below 130/80 mm Hg and the recognition that mean SBP levels of around 125 mm Hg are required to reach more than 80% of blood pressure levels below 140/90 mm Hg. Conversely, the trial included comparatively few participants with very high levels of blood pressure, which is an important minority of the population with hypertension. This makes direct comparison with previous trials challenging, because the extent of blood pressure reduction is strongly associated with the initial blood pressure level.

In conclusion, a novel triple-combination SPC of telmisartan, amlodipine, and indapamide at half and standard doses was more effective in lowering blood pressure than dual combinations and was well tolerated. Increased availability of SPCs can be expected to improve adherence compared with separate pills, and interventions such as the one tested here can facilitate clinically relevant improvements in efficacy for the large number of people with hypertension whose blood pressure remains uncontrolled after dual-combination therapy.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

1-24. (canceled)

25. A method of treating a subject having hypertension, the method comprising administering to the subject a pharmaceutical composition comprising:
(a) telmisartan;
(b) indapamide; and
(c) amlodipine besylate,
wherein said pharmaceutical composition is not administered in a subject who is prescribed aliskiren.

26. The method of claim 25, wherein the pharmaceutical composition is formulated for oral administration.

27. The method of claim 26, wherein the pharmaceutical composition is in the form of a pill, tablet, or capsule.

28. The method of claim 25, wherein the treating the subject results in a reduction of systolic blood pressure (SBP) in the subject of about 10 mmHg or greater.

29. The method of claim 25, wherein the treating the subject results in a reduction of diastolic blood pressure (DBP) of about 5 mmHg or greater.

30. The method of claim 25, wherein the treating the subject results in a reduction in SBP or DBP that is greater than the reduction obtained with the full lowest hypertension therapeutic dose of any one of the (a), (b), and (c) in the pharmaceutical composition.

31. The method of claim 25, wherein the treating the subject results in greater long-term tolerability and reduced risk of side effects when compared to treatment with the full lowest hypertension therapeutic dose of any one of (a), (b), and (c) in the pharmaceutical composition.

32. The method of claim 25, wherein a dose of the indapamide is about 0.625 mg.

33. The method of claim 25, wherein a dose of the amlodipine besylate is about EQ 1.25 mg base.

34. The method of claim 25, wherein a dose of the telmisartan is about 10 mg.

35. The method of claim 25, wherein a dose of the indapamide is about 0.625 mg, a dose of amlodipine besylate is about EQ 1.25 mg base, and a dose of the telmisartan is about 10 mg.

36. The method of claim 25, wherein a dose of the indapamide is about 1.25 mg.

37. The method of claim 25, wherein a dose of the amlodipine besylate is about EQ 2.5 mg base.

38. The method of claim 25, wherein a dose of the telmisartan is about 20 mg.

39. The method of claim 25, wherein a dose of the indapamide is about 1.25 mg, a dose of the amlodipine besylate is about EQ 2.5 mg base, and a dose of the telmisartan is about 20 mg.

40. The method of claim 25, wherein a dose of the indapamide is about 2.5 mg.

41. The method of any one of claim 25, wherein a dose of amlodipine besylate is about EQ 5 mg base.

42. The method of claim 25, wherein a dose of telmisartan is about 40 mg.

43. The method of claim 25, wherein a dose of the indapamide is about 2.5 mg, a dose of the amlodipine besylate is about EQ 5 mg base, and a dose of the telmisartan is about 40 mg.
